(19) United States
(12) Patent Application Publication
(10) Pub. No.: US 2020/0190136 A1
(43) Pub. Date: Jun. 18, 2020

(54) METHODS FOR GENERATING SMALL MOLECULE DEGRADERS AND DIMERIZERS

(71) Applicant: DANA-FARBER CANCER INSTITUTE, INC., Boston, MA (US)

(72) Inventors: Eric Fischer, Newton, MA (US); Radoslaw Nowak, Boston, MA (US)

(73) Assignee: DANA-FARBER CANCER INSTITUTE, INC., Boston, MA (US)

(21) Appl. No.: 16/620,353

(22) PCT Filed: Jun. 7, 2018
(86) PCT No.: PCT/US2018/036487

§ 371 Date: Dec. 6, 2019

Related U.S. Application Data
(60) Provisional application No. 62/517,500, filed on Jun. 9, 2017.
(60) Provisional application No. 62/575,059, filed on Oct. 20, 2017.

(51) Int. Cl. C07K 1/00; G01N 33/68

(52) U.S. Cl. CPC C07K 1/00; G01N 33/6845

(57) ABSTRACT

A method for generating a dimerization and/or degradation moiety for a first protein and a second protein, where the method includes (a) generating, in silico a set of poses by docking a first protein, and a second protein, (b) generating a subset of poses by selecting one or more poses from the set of poses based on the scores of the poses, (c) identifying a candidate pose from the subset of poses based on the spatial relationship between the two proteins, (d) designing a linker between the first ligand and the second ligand that accommodates the candidate pose, and (e) synthesizing or having synthesized the dimerization and/or degradation moiety having the first ligand, the second ligand, and the linker.

### RELATED APPLICATIONS

This application claims the benefit of priority under 35 U.S.C. § 119(e) to U.S. Provisional Application No. 62/517,500, filed Jun. 9, 2017 and to U.S. Provisional Application No. 62/575,059, filed Oct. 20, 2017, each of which is incorporated herein by reference in its entirety.

### GOVERNMENT LICENSE RIGHTS

This invention was made with government support under R01 CA214608 awarded by the National Institutes of Health. The government has certain rights in the invention.

### FIELD OF THE INVENTION

The present invention generally relates to methods for generating small molecules inducing dimerization, either in the form of heterobifunctional binders, molecular glues, or immunomodulatory imide drug (IMiD)-like glues, and more specifically to methods for generating small molecule degraders (also known as PROTACs, degraders, molecular glues, etc.), which can be of bifunctional nature.

### BACKGROUND

While long sought after, rational design of synthetic chemical matter capable to induce selective protein dimerization is challenging. Significant progress, has recently been made towards chemically induced targeted protein degradation using heterobifunctional compounds (small molecule ligands often referred to as degraders or PROTACs for PROteolysis-TArgeting Chimeras) (Bondeson et al., 2015; Buckley et al., 2015; Gadd et al., 2017; Gustafson et al., 2015; Kenten & Roberts, 2001; J. Lu et al., 2015; Sakamoto et al., 2001; Winter et al., 2015). Targeted protein degradation refers to small molecule induced ubiquitination and degradation of disease targets, in which a small molecule simultaneously recruits both a ubiquitin E3 ligase and the target protein to be ubiquitinated; therefore representing a functional application of chemically induced protein dimerization (Kenten & Roberts, 2001). Clinical proof of concept for targeted protein degradation is provided by the recent discovery that the potent anti-cancer drugs thalidomide, lenalidomide and pomalidomide (collectively known as IMiDs) exert their therapeutic effects through induced degradation of key efficacy targets, such as IKZF1, IKZF3 (Gandhi et al., 2014; Kronke et al., 2014; G. Lu et al., 2014), ZFP91 (An et al., 2017), or caseine kinase 1 alpha (Ck1α) (Kronke et al., 2015; G. Petzold, Fischer, & Thoma, 2016). IMiDs bind CRBN, the substrate receptor of the CUL4-RBX1-DDB1-CRBN (CRL4^{CRBN}) E3 ubiquitin ligase (Chamberlain et al., 2014; Fischer et al., 2014; Ito et al., 2010), and act by redirecting the activity of the CRL4^{CRBN} ligase to ubiquitinate these neo-substrates (G. Petzold et al., 2016) in a molecular glue-like fashion.

Heterobifunctional PROTACs (or degraders) typically comprise an E3 ligase binding scaffold (hereafter E3-moiety), often an analogue of thalidomide, or a ligand to the von Hippel-Lindau tumor suppressor (VHL) protein (Buckley et al., 2012), attached through a linker to another small molecule (hereafter target-moiety) that binds a target protein of interest (FIG. 1A and FIGS. 7A and B). Recruitment of this target protein to the E3 ubiquitin ligase facilitates ubiquitination and subsequent degradation of the target protein (Raina & Crews, 2017). This principle has been successfully applied to several targets including the Bromodomain and Extra Terminal (BET) family (BRD2, BRD3, BRD4), RIPK2, BCR-ABL, FKBP12, BRD9, and ERRa (Bondeson et al., 2015; Lai et al., 2016; J. Lu et al., 2015; Raina et al., 2016; Remillard et al., 2017; Toure & Crews, 2016; Winter et al., 2015) and is a promising new pharmacologic modality now widely explored in chemical biology and drug discovery.

Small molecule induced protein degradation by PROTACs or other small molecules, requires ligand mediated binding of two proteins that have not evolved to interact. While this is evidently possible, the design of such molecules remains an empirical process in which molecules for new targets frequently fail, likely due to insufficient understanding of the fundamental principles that govern these neo-interactions. Our structural understanding is limited to the recruitment of the second bromodomain of BRD4 (BRD4_{BD2}) to the CUL2-RBX1-ElongB/C-VHL (CRL2^{VHL}) ubiquitin ligase by the small molecule MZ1 (Gadd et al., 2017), a PROTAC based on a VHL-ligand (Buckley et al., 2012) conjugated to the BRD4 ligand JQ1 (Filippakopoulos et al., 2010). In this case, positive cooperativity was observed for VHL-MZ1-BRD2/3/4BD2 complex formation, where additional contacts between VHL and BRD4_{BD2} as well as back-folding of the linker with additional linker-ligase/substrate contacts result in superior affinity (linkage cooperativity) over the individual affinities for the VHL and BRD4 binding moieties (Douglass, Miller, Sparer, Shapiro, & Spiegel, 2013). Whether such tight PROTAC complexes are common and whether attractive inter-protein forces are required for effective degradation of target proteins that have not evolved to bind to the ligase is unknown.

Other PROTACs targeting BRD4 utilize the CRL4^{CRBN} targeting thalidomide moiety and it remains to be shown if these exhibit a similar ligase-substrate interface. In general, PROTACs have been found to exhibit different efficacy and selectivity profiles depending on the nature of the E3-moiety used, often exhibiting improved selectivity over the parental target-moiety (Zengerle, Chan, & Ciulli, 2015). While positive cooperativity can explain certain cases such as MZ1, it is unlikely to exist for a broad number of ligase-substrate pairs and whether desired selectivity profiles can be achieved for highly homologous proteins such as BRD2/3/4 is unknown. Based upon these current limitations, there remains a need for heterobifunctional compounds (PROTACs) that can selectively target a target protein, especially, over highly homologous related proteins.

Based upon these limitations, prior to the invention described herein, there was a need for improved methods for generating small molecule degraders and dimerizers (e.g., heterobifunctional and glue-like).

### SUMMARY OF THE INVENTION

191 The present invention is based, at least in part, upon the discovery and development of new and improved methods for generating small molecules that induce protein dimerization and/or protein degradation. The dimerization and/or degradation moiety may include a heterobifunctional binder (e.g., a PROTAC), a molecular glue, an immunomodulatory imide drug (IMiD)-like molecule/molecular glue, e.g., auxin/jasmonate, a cyclic peptide-like molecule, e.g., rapamycin, a peptide, a peptide mimetic, deoxyribonucleic acid (DNA), ribonucleic acid (RNA), a nucleic acid mimetic, and a “mini-protein,” e.g., a computationally-designed protein. For example, suitable dimerization and/or degradation moieties include zinc-finger-containing proteins and zinc-finger transcription factors, e.g., ikaros, aiolos, helios, and zfp91. For example, the methods provide docking to CRBN in the presence or absence of IMiDs and analogs of IMiDs (as shown herein for Ck1 and lenalidomide).

The dimerization and/or degradation moiety can be small molecule, or low molecular weight, compounds that bind, and promote interaction between, two proteins. The two proteins do not necessarily interact and/or bind in vivo. The interaction can cause a functional result such as an enzymatic activity, chemical modification, dimerization of the first and second protein, or degradation of at least one of the proteins.

In various embodiments, the methods can be used for generating small molecule dimerization and/or degradation moieties, e.g., heterobifunctional degraders, Proteolysis Targeting Chimeras (PROTACs) or degronimids. However, the methods are also generally applicable to generating dimerization and/or degradation moieties (e.g., heterobifunctional binders) for a first protein having a first ligand and a second protein having a second ligand. The methods can be used to create libraries of dimerization and/or degradation moieties and/or screen dimerization and/or degradation moieties such as heterobifunctional binders (e.g., for drug discovery, development). The methods can be used to assess/predict the suitability of a target to ligand for inducing protein dimerization and/or protein degradation. The methods can be used to screen and/or interrogate protein interactions and function. Examples of heterobifunctional binders and libraries of heterobifunctional binders are described, for example, in US Patent Application Publication No. 2016/0176916 (U.S. Ser. No. 14/707,930). Suitable dimerization and/or degradation moieties include dimerizers and degraders, e.g., heterobifunctional binders, molecular glues, molecular glue-like molecules, and immunomodulatory drugs (IMiDs).

Without wishing to be bound by any particular theories, the heterobifunctional organization of degraders can confer unusual biochemical properties. Cellular efficacy of target degradation (represented as DC₅₀ values for the concentration providing 50% of maximal degradation) can exceed the degrader affinities for the ligase and target (Lu et al., 2015; Raina et al., 2016; Winter et al., 2015). Furthermore, changes to the linker or the ligase targeting moiety, can change target specificity, as seen for BRD2, 3, and 4 (Zengerle et al., 2015). The observed gain in selectivity of the degrader relative to its parent compound (Zengerle et al., 2015) suggests that protein-protein interactions (PPI) between the ligase and target may exist. Such inter-protein contacts could establish specific conformations or result in cooperativity and increased binding avidity, both of which can contribute to selectivity. The present invention exploits the existence of critical PPIs, and takes them into consideration in the rational design of novel binders (e.g., degraders).

In various aspects, the invention provides a method for generating a dimerization and/or degradation moiety (e.g., a heterobifunctional binder or glue-like molecule) for a first protein and a second protein. The method comprises (a) generating a first set of poses by docking a first protein structure and a second protein structure in silico; (b) generating a set of feasible poses by (i) selecting a subset of the first set poses by scoring and (ii) structurally clustering the subset in silico: (c) selecting a preferred pose from the set of feasible poses based upon the relative position and orientation of the first protein structure and the second protein structure; (d) designing a covalent linker between a first ligand for the first protein and a second ligand for the second protein in the preferred pose; and (e) synthesizing a dimerization and/or degradation moiety (e.g., a heterobifunctional binder) comprising the first ligand, the second ligand, and the covalent linker. The first and/or second ligand can be present in step (a), or can be added a later time (e.g., docked separately during step (d)).

In various aspects, the invention provides a method for generating a dimerization and/or degradation moiety (e.g., a heterobifunctional binder) for a first protein and a second protein. The method comprises (a) generating a first set of poses by docking a first protein-first ligand pair structure and a second protein-second ligand pair structure in silico: (b) generating a set of feasible poses by (i) selecting a subset of the first set poses by scoring and (ii) structurally clustering the subset in silico; (c) selecting a preferred pose from the set of feasible poses based upon the relative position and orientation of the first protein-first ligand pair structure and the second protein-second ligand pair: (d) designing a covalent linker between the first ligand and the second ligand in the preferred pose: and (e) synthesizing a dimerization and/or degradation moiety (e.g., a heterobifunctional binder) comprising the first ligand, the second ligand, and the covalent linker.

In various aspects, the invention provides a method for generating a dimerization and/or degradation moiety (e.g., a heterobifunctional binder) for a first protein and a second protein. The method comprises (a) generating, in silico, a set of poses by docking a first protein, optionally bound to a first ligand, and a second protein, optionally bound to a second ligand, where (i) a score is calculated based on energy of interactions between the first protein and the second protein for each of the poses; and (ii) a spatial relationship between the first protein and the second proteins is quantified for each of the poses, (b) generating a subset of poses by selecting one or more poses from the set of poses based on the scores of the poses, (c) identifying a candidate pose from the subset of poses based on the spatial relationship between the two proteins, (d) designing a linker between the first ligand and the second ligand that accommodates the candidate pose; and (e) synthesizing or having synthesized the dimerization and/or degradation moiety (e.g., heterobifunctional binder) having the first ligand, the second ligand, and the linker.

Design of selective degraders is prepared as follows. Structures (or homology models) of related (e.g., isoforms, homologs, potential-off targets) proteins are structurally aligned to their docked pose. Next, diversity hotspots are defined as locations of the protein sequence/structure with sequence diversity (such as, but not limited to, point mutations). Then, poses are identified for which diversity hotspots present themselves in the protein-protein interface. Hotspots present in the interface will likely disturb it, and potentially destabilize it, and resulting poses will favor certain mutations, translating to selective dimerization. Multiple docked poses may result in distinct interface hotspots, which can be explored to direct dimerization selectivity to the target. Design of non-selective degraders is achieved in the same method by in turn focusing on poses that have no hotspots in the protein-protein interface.

As will be understood by those skilled in the art, the aspect above can be combined with any one or more of the features below.

In various embodiments, the invention further comprises experimentally measuring binding of the first protein, the second protein, and the dimerization and/or degradation moiety (e.g., heterobifunctional binder).

In various embodiments, the invention further comprises experimentally measuring a functional result of binding the first protein, the second protein, and the dimerization and/or degradation moiety (e.g., heterobifunctional binder). The functional result comprises an enzymatic activity, chemical modification, dimerization of the first and second protein, or degradation of the first or second protein.

In various embodiments, the invention further comprises synthesizing a library of dimerization and/or degradation moieties (e.g., heterobifunctional binders).

In various embodiments, the invention further comprises experimentally screening the library of dimerization and/or degradation moieties (e.g., heterobifunctional binders).

In various embodiments, the step of synthesizing, measuring, or screening can include synthesizing, measuring, or screening carried out by a third party such as a collaborator or contractor. The step of synthesizing, measuring, or screening can include instructing/directing a third party to carry out the step of synthesizing, measuring, or screening.

In various embodiments, the first and second proteins do not naturally bind each other in vivo.

In various embodiments, the first protein or the second protein is a ubiquitin ligase. The ubiquitin ligase can be an E3 ubiquitin ligase or a component of an E3 ubiquitin ligase. The E3 ubiquitin ligase can be CRL4^{CRBN}, CRL4^{DCAF15}, CRL3^{KEAP1} or CRL2^{VHL}. The component of the E3 ubiquitin ligase can be CRBN, DCAF15, KEAP1, or VHL.

In various embodiments, the first protein or the second protein is an E2 ubiquitin conjugating enzyme.

In various embodiments, the first protein or the second protein is a Von Hippel-Lindau tumor suppressor protein (VHL).

In various embodiments, the first protein or the second protein is a subunit of a proteasome.

In various embodiments, the first ligand or the second ligand is a ubiquitin ligase ligand.

In various embodiments, the first ligand or the second ligand is an E3 ubiquitin ligase ligand.

In various embodiments, the first ligand or the second ligand is thalidomide, lenalidomide, pomalidomide, or an analog or derivative thereof.

In various embodiments, the first ligand or the second ligand is a E2 ubiquitin conjugating enzyme ligand.

In various embodiments, the first ligand or the second ligand is a Von Hippel-Lindau tumor suppressor protein (VHL) ligand.

In various embodiments, the first ligand or the second ligand is a proteasome subunit ligand.

In various embodiments, step (d) further comprises calculating a shortest path or shortest distance between the first and second ligands. The shortest path can be calculated between a centroid and/or a predetermined atom of each of the first and second ligands.

Shortest distance can be calculated as minimum Euclidean distance between a centroid and/or a predetermined atom of each of the first and second ligands.

In various embodiments, the invention further comprises fitting a chemical structure to the shortest path or shortest distance, thereby designing the covalent linker.

In various embodiments, the preferred pose comprises a set of preferred poses.

In various embodiments, the method comprises designing a set of heterobifunctional binders. The set of heterobifunctional binders can correspond to the set of preferred poses.

In various embodiments, step (d) further comprises docking a first ligand to the first protein and/or a second ligand to the second protein (e.g., where the first and/or second ligand is not docked in step (a) or where the first and/or second ligand is changed in step (d) or where the first and/or second ligand structure is refined in step (d)).

In various embodiments, the dimerization and/or degradation moiety (e.g., heterobifunctional binder) causes degradation of the first protein with a higher specificity than the binding specificity of the first ligand for the first protein.

In various embodiments, the spatial relationship between the first protein and the second protein is quantified by calculating the shortest path or shortest distance between a first set of solvent-exposed atoms on the first ligand and a second set of solvent-exposed atoms on the second ligand.

In various embodiments, the spatial relationship between the first protein and the second protein is quantified by calculating the shortest path or shortest distance between the centroid of the first ligand and the centroid of the second ligands. In various embodiments, the dimerization and/or degradation moiety (e.g., heterobifunctional binder) dimerizes the first protein and the second protein in a low-energy level conformation.

In various embodiments, the plurality of shortest paths calculated is compiled to generate a distance profile for the subset of poses.

In various embodiments, the distance profile of the subset of poses has a distinct cluster of poses that have similar shortest paths. In various embodiments, the candidate pose is the lowest scoring pose of the cluster of poses.

In various embodiments, the specificity of the dimerization and/or degradation moiety (e.g., heterobifunctional binder) for the first protein and the second protein is predicted from the distance profile for the subset of poses.

In various embodiments, relative specificity the dimerization and/or degradation moiety (e.g., heterobifunctional binder) for two different first proteins can be predictively distinguished by comparing the distance profiles for the subset of poses for each of the two different first proteins and the second protein.

In various embodiments, the method further comprises experimentally measuring binding of the first protein, the second protein, and the dimerization and/or degradation moiety (e.g., heterobifunctional binder).

In various embodiments, the method further comprises experimentally measuring a functional result of binding the first protein, the second protein, and the dimerization and/or degradation moiety (e.g., heterobifunctional binder).

In various embodiments, the functional result comprises an enzymatic activity, chemical modification, dimerization of the first and second protein, or degradation of the first or second protein.

In various embodiments, the method further comprises synthesizing a library of dimerization and/or degradation moieties (e.g., heterobifunctional binders).

In various embodiments, the method further comprising experimentally screening the library of dimerization and/or degradation moiety (e.g., heterobifunctional binders).

In various embodiments, the first and second proteins do not naturally bind each other in vivo.

In various embodiments, the first protein or the second protein is a ubiquitin ligase.

In various embodiments, the ubiquitin ligase is an E3 ubiquitin ligase.

In various embodiments, the ubiquitin ligase is a component of an E3 ubiquitin ligase.

In various embodiments, the first protein or the second protein is an E2 ubiquitin conjugating enzyme.

In various embodiments, the first protein or the second protein is CRL2^{VHL}.

In various embodiments, the first protein or the second protein is a subunit of a proteasome.

In various embodiments, the first ligand or the second ligand is a ubiquitin ligase ligand.

In various embodiments, the first ligand or the second ligand is an E3 ubiquitin ligase ligand.

In various embodiments, the first ligand or the second ligand is a ligand for a component of the E3 ubiquitin ligase.

In various embodiments, the first ligand or the second ligand is thalidomide, lenalidomide, pomalidomide, or an analogue or derivative thereof.

In various embodiments, the first ligand or the second ligand is a E2 ubiquitin conjugating enzyme ligand.

In various embodiments, the first ligand or the second ligand is a Von Hippel-Lindau tumor suppressor protein (VHL) ligand.

In various embodiments, the first ligand or the second ligand is a proteasome subunit ligand.

In various embodiments, the step of designing the linker further comprises fitting a chemical structure to the shortest path of the candidate pose, thereby designing the linker.

Also provided are methods of designing selective degraders based upon family-wide protein sequence alignment of close homologues (potential off-targets).

These and other advantages of the present technology will be apparent when reference is made to the accompanying drawings and the following description.

### BRIEF DESCRIPTION OF THE DRAWINGS

These and other advantages of the present technology will be apparent when reference is made to the following description.

FIG. 1A-FIG. 1D show the overall structure of the DDB1ΔB-CRBN-dBET23-BRD4_{BD1} complex.

FIG. 1A shows the chemical structure of dBET23 with the target-moiety in red, the linker in black and green, and the E3-moiety in blue.

FIG. 1B shows a cartoon representation of DDB1ΔB-CRBN-dBET23-BRD4_{BD1}: DDB1 highlighting domains BPA (red), BPC (orange) and DDB1-CTD (grey); CRBN with domains NTD (blue), HBD (cyan) and CTD (green); and BRD4_{BD1} (magenta). The Zn²⁺-ion is shown as a grey sphere and dBET23 as sticks representation in yellow. The F_{O}-F_{C} map is shown as green mesh for dBET23 contoured at 3.0σ.

FIG. 1C shows superposition of DDB1ΔB-CRBN-dBET23-BRD4_{BD1} with human CRBN bound to lenalidomide (PDB: 4tz4) and BRD4_{BD1} bound to JQ1-(S) (PDB: 3mxf). Surface representation for CRBN and BRD4_{BD1} are shown in grey and magenta, respectively, dBET23 is shown in yellow, JQ1 in green, and thalidomide in cyan.

FIG. 1D shows side-chain interactions between BRD4_{BD1}, CRBN, and dBET23. Dashed lines indicate hydrogen bonds. Residues of BRD4_{BD1} mutated in this study are highlighted in cyan.

FIG. 2A-FIG. 2F show data demonstrating that dBET mediated BRD4 recruitment is governed by negative cooperativity. All data in FIGS. 2A, C, and D represent biological replicates presented as means±s.d. (n=3).

FIG. 2A shows TR-FRET data where dBET23 is titrated to DDB1ΔB-CRBN_{SPY-BODIPY}, Terbium-Streptavidin and various BRD4_{BD1-biotin} wild type and mutant proteins. The mean peak heights for dose response curves of three independent replicates are shown as bar charts.

FIG. 2B shows surface representation of CRBN highlighting the residues involved in dBET23 mediated BRD4_{BD1} binding in orange.

FIG. 2C shows competitive binding assay for dBET1 binding to DDB1ΔB-CRBN. Increasing concentrations of dBET1 titrated to preformed DDB1ΔB-CRBN-lenalidomide_{Atto565} complex in presence or absence of BRD4_{BD1} or BRD4_{BD2} are shown.

FIG. 2D, FIG. 2E, and FIG. 2F show similar competitive assays for dBET6, dBET23 and dBET57, respectively.

FIG. 3A-FIG. 3F show quantitative assessment of cellular degradation for BRD4_{BD1} and BRD4_{BD2}.

FIG. 3A, FIG. 3B, and FIG. 3C show quantitative assessment of cellular degradation using a BRD4_{BD1}-EGFP reporter assay. Cells stably expressing BRD4_{BD1}-EGFP and mCherry were treated with increasing concentrations of lenalidomide, dBET1, dBET6, dBET23, dBET55, dBET57, dBET70, and MZ1 and the EGFP and mCherry signals followed using flow cytometry analysis.

FIG. 3D, FIG. 3E, and FIG. 3F show quantitative assessment of cellular degradation using a BRD4_{BD2}-EGFP reporter assay. Cells stably expressing BRD4_{BD2}-EGFP and mCherry were treated with increasing concentrations of dBET1, dBET6, dBET23, dBET55, dBET57, dBET70, MZ1 and lenalidomide. EGFP and mCherry signals were measured using flow cytometry analysis.

Data in FIG. 3A-FIG. 3F represent four biological replicates analyzed in technical duplicates with 5000 cells each, and presented as the means±s.d.

FIG. 4A-FIG. 4H show data demonstrating plasticity of CRBN-substrate interactions.

FIG. 4A shows TR-FRET data where dBET23 is titrated to BRD4_{BD1-SPYCATCHER-BODIPY}, Terbium-antiHis antibody and various His6-DDB1ΔB-CRBN wild type and His6-DDB1-CRBN mutant proteins. The mean peak heights for dose response curves of three independent replicates are shown as bar charts.

FIG. 4B shows TR-FRET data where dBET23 is titrated to DDB1ΔB-CRBN_{SPYCATCHER-BODIPY}, Terbium-Streptavidin and various BRD4_{BD1-biotin} wild type and mutant proteins. The mean peak heights for dose response curves of three independent replicates are shown as bar charts.

FIG. 4C shows TR-FRET data where dBET57 is titrated to BRD4_{BD1-SPYCATCHER-BODIPY}, Terbium-antiHis antibody and various His6-DDB1ΔB-CRBN wild type and His6-DDB1-CRBN mutant proteins.

FIG. 4D shows TR-FRET data where dBET57 is titrated to DDB1ΔB-CRBN_{SPYCATCHER-BODIPY}, Terbium-Streptavidin and various BRD4_{BD1-biotin} wild type and mutant proteins. Data in FIGS. 4A-FIG. 4D represent biological replicates presented as means±s.d. (n=3).

FIG. 4E shows the chemical structure of dBET57 with the target-moiety in red, the linker in black and green, and the E3-moiety in blue.

FIG. 4F shows a cartoon representation of DDB1ΔB-CRBN-dBET57-BRD4_{BD1}: DDB1 highlighting domains BPA (red), BPC (orange) and DDB1-CTD (grey); CRBN with domains NTD (blue), HBD (cyan) and CTD (green); BRD4_{BD1} (magenta). The Zn²⁺-ion is drawn as a grey sphere, dBET57 was not modelled in this structure but instead superpositions of lenalidomide (from pdb: 5fqd) and JQ1 (from pdb: 3mxf) are shown in yellow sticks.

FIG. 4G shows superposition of CRBN and BRD4_{BD1} for the dBET23 and dBET57 containing complexes. Superposition was carried out over the CRBN-CTD (residues 320-400).

FIG. 4H shows surface representation of CRBN highlighting the BRD4_{BD1} interacting residues for the dBET57 mediated recruitment in orange.

FIG. 5A-FIG. 5C show in silico docking to predict binding modes.

FIG. 5A shows symmetric docking energy landscape for the binding of BRD4_{BD1} to a CRBN-lenalidomide complex. The two low energy decoys that exhibit a conformation compatible with dBET binding are indicated by bold numbers. The symmetric docking energy landscape for local perturbation docking experiments on decoy 12662 compatible with dBET mediated binding is shown as insert.

FIG. 5B shows superposition of the DDB1ΔB-CRBN-dBET23-BRD4_{BD1} structure and the top solution from local perturbation of decoy 12662.

FIG. 5C shows cartoon representations of three representative clusters from the global docking run.

FIG. 6A-FIG. 6H show data demonstrating degradation of BET family proteins by certain heterobifunctional small molecule degraders.

FIG. 6A shows a cartoon representation of structures from cluster 19, and close-up view highlighting the proximity of the JQ1 thiophene and lenalidomide that provided the rationale for synthesizing the heterobifunctional small molecule degrader ZXH-03-26, which is shown in FIG. 6B.

FIG. 6C shows quantitative assessment of cellular degradation using a EGFP/mCherry reporter assay. Cells stably expressing BRD4_{BD1}-EGFP (or constructs harbouring BRD2_{BD1}, BRD2_{BD2}, BRD3_{BD1}, BRD3_{BD2}, BRD4_{BD2}) and mCherry were treated with increasing concentrations of ZXH-03-26 and the EGFP and mCherry signals followed using flow cytometry analysis.

FIG. 6D-FIG. 6F show quantitative assessment of cellular degradation using a EGFP/mCherry reporter assay. Cells stably expressing BRD4_{BD1}-EGFP (or constructs harbouring BRD2_{BD1}, BRD2_{BD2}, BRD3_{BD1}, BRD3_{BD2}, BRD4_{BD2}) and mCherry were treated with increasing concentrations dBET6 (FIG. 6D), MZ1 (FIG. 6E), and dBET57 (FIG. 6F).

FIG. 6G shows data demonstrating cellular degradation of endogenous BRD4 in HEK293T cells that were treated with increasing concentrations of ZXH-03-26 or dBET6 for 5 hours, and protein levels assessed by western blot.

FIG. 6H shows degradation of BRD2 and BRD3 by western blot.

FIG. 7A-FIG. 7E show structure of the DDB1ΔB-CRBN-dBET23-BRD4_{BD1} complex.

FIG. 7A shows a schematic representation of the heterobifunctional ligand (PROTAC/degrader) mediated degradation.

FIG. 7B shows chemical structures, molecular weight and C Log P for the heterobifunctional small molecule degraders (BET inhibitor JQ1-(S) coloured in red, thalidomide moiety coloured in blue and the linker in black and green).

FIG. 7C shows multiple sequence alignment of BD1 and BD2 from different BET bromodomain paralogs. (SEQ ID Nos: 1-8 in order of appearance.)

FIG. 7D shows multiple sequence alignment of BD1 and BD2 from human BRD4. (SEQ ID Nos: 9-10 in order of appearance.)

FIG. 7E shows domain architecture of BDR4 (A and B—DNA binding motifs; ET—external domain; SEED—Ser/Glu/Asp-rich region; CTM—C-terminal domain).

FIGS. 8A-FIG. 8J show structures of dBET6, dBET70 and dBET55 complexes. FIG. 8A shows a cartoon representation of DDB1ΔB-CRBN-dBET6-BRD4_{BD1}. The F_{O}-F_{C} map is shown as green mesh for dBET6 contoured at 4.0.

FIG. 8B shows a cartoon representation of DDB1ΔB-CRBN-dBET70-BRD4_{BD1}. The F_{O}-F_{C} map is shown as green mesh for dBET70 contoured at 4.0σ.

FIG. 8C shows a cartoon representation of DDB1ΔB-CRBN-dBET55-BRD4_{BD1/D14A}. The F_{O}-F_{C} map is shown as green mesh contoured at 3.0σ. In FIGS. 8A-C, DDB1 is shown in grey, CRBN in blue, and BRD4_{BD1} (wildtype and mutant) in magenta.

FIGS. 8D-FIG. 8J show TR-FRET data underlying bar charts shown in FIG. 2A, FIG. 4A-FIG. 4D and FIG. 11D-FIG. 11L. The TR-FRET data in FIGS. 8D-FIG. 8J represent biological replicates presented as means±s.d. (n=3).

FIG. 9A-FIG. 9H show data demonstrating negative cooperativity governing CRBN-dBET-BRD4 interactions.

FIG. 9A shows a schematic of fluorescence polarization based CRBN binding assay. Atto565-Lenalidomide fluorophore is displaced by PROTAC bound BRD4_{BD1/2}.

FIG. 9B shows fluorescence polarization competitive binding assay for dBET55 binding to DDB1ΔB-CRBN. Increasing concentrations of dBET55 titrated to preformed DDB1ΔB-CRBN-lenalidomide_{Atto565} complex in presence or absence of BRD4_{BD1} or BRD4_{BD2}.

FIG. 9C-FIG. 9G show fluorescence polarization competitive binding assay for dBET1, dBET6, dBET23, dBET55, and dBET57, respectively, to DDB1ΔB-CRBN with increasing concentrations of dBETs titrated to preformed DDB1ΔB-CRBN-lenalidomide_{Atto565} complex in presence or absence of BRD4_{BD1} or BRD4_{BD2} at concentrations of 1 μM, 5 μM, and 20 μM. The data at 5 μM BRD4_{BD1/2} was replotted for FIGS. 2C-F and FIG. 9B.

FIG. 9H shows summary of apparent cooperativity factors αₐₚₚ.

FIG. 10A, FIG. 10B, FIG. 10C, FIG. 10D, FIG. 10E, FIG. 10F, FIG. 10G, FIG. 10H, FIG. 10I, FIG. 10J, FIG. 10K, and FIG. 10L show quantitative assessment of cellular degradation of BRD4_{BD1}-EGFP/BRD4_{BD2}-EGFP and IKZF1Δ-EGFP by lenalidomide, dBET1, dBET6, dBET23, dBET55, dBET57, dBET70, dBET72, MZ1, ZXH-2-42, ZXH-2-43, and ZXH-2-45, respectively, using flow cytometry analysis. Cells stably expressing BRD4_{BD1}-EGFP/BRD4_{BD2}-EGFP or IKZF1Δ-EGFP with a mCherry reporter were treated with increasing concentrations of the heterobifunctional small molecule degraders with the EGFP and mCherry signals quantified using flow cytometry analysis.

FIG. 11A-FIG. 11I show plasticity of CRBN-substrate interactions.

FIG. 11A shows the different surfaces CRBN utilizes to interact with a variety with neo-substrates as illustrated by the superposition of DDB1ΔB-CRBN-dBET23-BRD4_{BD1}, DDB1ΔB-CRBN-lenalidomide-Ck1α (PDB entry 5fqd), and DDB1-CRBN-CC885-GSPT1 (PDB entry 5hxb). Close-up of the common hydrophobic interface between GSPT1-CRBN-NTD and BRD4_{BD1}-CRBN-NTD is shown in the top right box.

FIG. 11B shows a competitive binding assay where titrating BRD4_{BD1} or BRD4_{BD2} into a preformed complex of DDB1-CRBN-dBET57-IKZF1Δ demonstrated mutually exclusive binding of BRD4 with neosubstrates such as Ck1α or IKZF1/3.

FIG. 11C shows a surface representation of CRBN and BRD4_{BD1} of DDB1-CRBN-dBET23-BRD4_{BD1} crystal structure, showing dBET23 as stick representation. The hypothetical linker path from the acid position on JQ1 is shown with red spheres indicating the distance of a carbon-carbon bond and illustrating that the 2-carbon linker of dBET57 would be insufficient to bridge the gap.

FIG. 11D shows TR-FRET data where dBET6 degrader was titrated to BRD4_{BD1SPYCATCHER-BODIPY} Terbium-antiHis antibody, and wild type or various mutants of His6-DDB1-His6-CRBN complex. The peak height of the dose response curve for three independent replicates was quantified and is depicted as bar charts. The TR-FRET data in this figure are biological replicates presented as means±s.d. (n=3).

FIG. 11F and FIG. 11H show TR-FRET data where dBET and dBET55, respectively, were titrated to BRD4_{BD1SPYCATCHER-BODIPY} Terbium-antiHis antibody, and wild type or various mutants of His6-DDB1-His6-CRBN complex. The peak height of the dose response curve for three independent replicates was quantified and is depicted as bar charts. The TR-FRET data in this figure are biological replicates presented as means±s.d. (n=3).

FIG. 11E shows TR-FRET data where dBET6 degrader was titrated to DDB1ΔB-CRBN_{SPYCATCHER-BODIPY}, Terbium-Streptavidin and wild type or mutants of BRD4_{BD1-biotin}. The peak height of the dose response curve for three independent replicates was quantified and is depicted as bar charts. The TR-FRET data in this figure are biological replicates presented as means±s.d. (n=3).

FIG. 11G and FIG. 11I show TR-FRET data where dBET and dBET55, respectively, were titrated to BRD4_{BDS1PYCATCHER-BODIPY}, Terbium-antiHis antibody, and wild type or various mutants of His6-DDB1-His6-CRBN complex. The peak height of the dose response curve for three independent replicates was quantified and is depicted as bar charts. The TR-FRET data in this figure are biological replicates presented as means±s.d. (n=3).

FIG. 12A-FIG. 12C show experimental validation of DDB1-CRBN-dBET57-BRD4_{BD1} structure.

FIG. 12A shows a cartoon representation of DDB1-CRBN-dBET57-BRD4_{BD1} complex with the 2F_{O}-F_{C} map contoured at 1.5σ. Domains are coloured as DDB1-BPA (red), DDB1-BPC (orange), DDB1-CTD (grey), CRBN-NTD (blue), CRBN-HBD (cyan), CRBN-CTD (green), and BRD4_{BD1} (magenta).

FIG. 12B shows anomalous difference map contoured at 40 shown in green for data collected at the Zn peak showing the position of the Zn in the final model. 2 F_{O}-F_{C} map is shown as blue mesh. FIG. 12C shows F_{O}-F_{C} map contoured at 3.50 and shown in green and red, together with 2 F_{O}-F_{C} map contoured at 1.5σ and shown in blue. Positive difference density is observed for the Thalidomide (Thal) and JQ1 binding sites.

FIG. 13A-FIG. 13D show in silico docking of CRBN-lenalidomide-Ck1 complex, i.e., molecular glue docking.

FIG. 13A shows symmetric docking energy landscape for the binding of Ck1α to a CRBN-lenalidomide complex. Symmetric docking energy landscape for local perturbation docking experiments on a lowest energy decoy 00689 is shown as insert.

FIG. 13B shows superposition of the DDB1ΔB-CRBN-lenalidomide-Ck1α structure (PDB: 5fqd) and the top solution, decoy 0173, from FIG. 13A.

FIG. 13C shows symmetric energy docking landscape for the binding of Ck1α to a CRBN-lenalidomide complex. The conformer parameter file for lenalidomide was restricted to a conformer not favorable of Ck1α binding.

FIG. 13D shows superposition of the DDB1ΔB-CRBN-lenalidomide-Ck1α structure (PDB: 5fqd) and the top solution from FIG. 13C.

FIG. 14A-FIG. 14E show co-degradation of IMiD neo-substrates such as IKZF1/3.

FIG. 14A shows TR-FRET data where titration of the indicated molecules to DDB1ΔB-CRBN_{SPYCATCHER-BODIPY}, Terbium-streptavidin and IKZF1Δ_{biotin}. Data in this figure are presented as means±s.d. (n=3).

FIG. 14B shows quantitative assessment of cellular degradation of a IKZF1-EGFP reporter using flow cytometry analysis. Cells stably expressing IKZF1Δ-EGFP and mCherry were treated with increasing concentrations of the indicated molecules and the EGFP and mCherry signals followed using flow cytometry analysis. Data in this figure are presented as means±s.d. (n=4).

FIG. 14C shows a model of a CRBN-IKZF1ZnF2 complex (adapted from Petzold et al., 2016) bound to lenalidomide. Potential hydrogen bonds are indicated as dashed lines.

FIG. 14D shows scatter plot depicting the fold changes in relative abundance comparing dBET23 to DMSO control treatment (MM.1s) determined using quantitative proteomics. Negative false discovery rate adjusted P Values are shown on the x-axis and log 2 fold changes on the y-axis. Data shown are three biological replicates measured in a single 10-plex TMT experiment.

FIG. 14E shows similar experiment as FIG. 14D but for dBET70 to DMSO control.

FIG. 15A-FIG. 15C show selective degradation of BRD4 by certain heterobifunctional small molecule degraders ZXH-3-147 and 184, as compared to non-selective degradation of BET family proteins by ZXH-3-27.

FIG. 15A shows selective degradation of BRD4 by ZXH-2-147 using quantitative assessment of cellular degradation using EGFP/mCherry reporter assay. Cells stably expressing BRD4_{BD1}-EGFP (or constructs harbouring BRD2_{BD1}, BRD2_{BD2}, BRD3_{BD1}, BRD3_{BD2}, BRD4_{BD2}) and mCherry were treated with increasing concentrations of ZXH-02-147 and the EGFP and mCherry signals followed using flow cytometry analysis.

FIG. 15B shows selective degradation of BRD4 by ZXH-2-184 using the same quantitative assessment as FIG. 15A.

FIG. 15C shows a lack of selective degradation of BRD4 by ZXH-3-27 using the same quantitative assessment as FIG. 15A.

FIG. 16A-FIG. 16L shows selective degradation of BRD4 by certain heterobifunctional small molecule degraders.

FIG. 16A, FIG. 16C, FIG. 16E, FIG. 16G, FIG. 16I, and FIG. 16K show chemical structures of ZXH-3-79, ZXH-3-27, ZXH-2-147, ZXH-2-184, ZXH-3-26, and ZXH-3-82.

FIG. 16B, FIG. 16D, FIG. 16F, FIG. 16H, FIG. 16J, and FIG. 16L show degradation of BRD4 by ZXH-3-79, ZXH-3-27, ZXH-2-147, ZXH-2-184, ZXH-3-26, and ZXH-3-82, respectively, via quantitative assessment of cellular degradation using EGFP/mCherry reporter assay. Cells stably expressing BRD4_{BD1}-EGFP (or constructs harbouring BRD2_{BD1}, BRD2_{BD2}, BRD3_{BD1}, BRD3_{BD2}, BRD4_{BD1}, BRD4_{BD2}) and mCherry were treated with increasing concentrations of ZXH-03-79 and the EGFP and mCherry signals followed using flow cytometry analysis.

FIG. 17A-FIG. 17I show TR-FRET data illustrating mutational profiles of various heterobifunctional compounds. TR-FRET data for dBET1 (FIG. 17A), dBET6 (FIG. 17B), dBET23 (FIG. 17.C), dBET55 (FIG. 17D), dBET57 (FIG. 17E), ZXH-3-26 (FIG. 17F and FIG. 17H) and dBET70 (FIG. 17G and FIG. 17I) titrated to DDB1ΔB-CRBN_{SPYCATCHER-BODIPY}, Terbium-Streptavidin and various BRD4_{BD1-biotin} wild type and mutant proteins are shown. The mean peak heights for dose response curves of three independent replicates are shown as bar charts. The TR-FRET data in FIGS. 17A-FIG. 17I represent biological replicates presented as means±s.d. (n=3).

FIG. 18 shows an example heterobifunctional binder development algorithm.

FIG. 19A and FIG. 19B show an example linker development algorithm. FIG. 19A shows an example shortest path calculation. FIG. 19B shows an example long path calculation.

FIG. 20 shows histogram of shortest pairwise distances found in docking poses between solvent exposed atoms of JQ1 bound to BRD4 BD1 and Lenalidomide bound to CRBN. Distances from 10,000 docking poses are shown in black and top 200 poses based on the docking score in gray.

FIG. 21A-FIG. 21B is a series of schematic diagrams and a graph showing in silico docking to design degrader molecules using the shortest distance algorithm. FIG. 21A is a cartoon showing representations for representative clusters obtained by k-means clustering of the top 200 global docking poses between CRBN (pdb: 4tz4) and BRD4_{BD1} (pdb: 3mxf). FIG. 21B is a histogram of the pairwise shortest distances for the top 200 docking poses. FIG. 21C is a schematic showing a close-up view on the proximity of the JQ1 thiophene and lenalidomide that provided the rationale for synthesizing ZXH-2-147 and ZXH-3-26. Atoms used for calculation of the pairwise shortest distances between JQ1 and lenalidomide are highlighted in black circles.

FIG. 22A-FIG. 22M is a series of graphs showing plasticity of CRBN-substrate interactions. As described herein, plasticity in binding confers selectivity in ligand induced protein degradation. Specifically, FIG. 22A-FIG. 22M show additional mutation data for ZXH-3-26 and dBET70 confirming distinct modes that these two molecules support. FIG. 22A is a schematic showing that CRBN utilizes different surfaces to interact with a variety with neo-substrates as illustrated by the superposition of DDB1ΔB-CRBN-dBET23-BRD4_{BD1}, DDB1ΔB-CRBN-lenalidomide-Ck1α (pdb: 5fqd), and DDB1-CRBN-CC885-GSPT1 (pdb: 5hxb). Top right, close-up of the common hydrophobic interface between GSPT1-CRBN-NTD and BRD4_{BD1}-CRBN-NTD. FIG. 22B is a line graph showing that the structures of DDB1-CRBN-dBET23-BRD4_{BD1} and DDB1-CRBN-lenalidomide-CK1a suggest mutually exclusive binding of BRD4 with neo-substrates such as Ck1α or IKZF1/3, which is confirmed by titrating BRD4_{BD1} or BRD4_{BD2} into a preformed complex of DDB1-CRBN-dBET57-IKZF1Δ. Data is presented as mean and standard deviation of 10 technical replicates of a single experiment (n=1). FIG. 22C is a schematic showing the surface representation of CRBN and BRD4_{BD1} of DDB1-CRBN-dBET23-BRD4_{BD1} crystal structure, showing dBET23 as stick representation. The hypothetical linker path from the acid position on JQ1 is shown with red spheres indicating the distance of a carbon-carbon bond and illustrating that the 2-carbon linker of dBET57 would be insufficient to bridge the gap. FIG. 22D is a graph showing TR-FRET. ZXH-3-26 degrader titrated to BRD4_{BD1}-SPYCATCHER-BODIPY and Terbium-antiHis antibody, and wild type or various mutants of His6-DDB1-His6-CRBN complex. The peak height of the dose response curve for three independent replicates was quantified and is depicted as dot-plot. TR-FRET data in this figure are independent replicates presented as means±s.d. (n=3). (FIG. 22E, FIG. 22H, FIG. 22I, and FIG. 22J) as in FIG. 22D, but for dBET70, dBET6, dBET1 and dBET55, respectively. FIG. 22F is a graph showing TR-FRET. ZXH-3-26 degrader titrated to DDB1ΔB-CRBN_{SPYCATCHER-BODIPY}, Terbium-Streptavidin and wild type or mutants of BRD4_{BD1-biotin}. The peak height of the dose response curve for three independent replicates was quantified and is depicted as dot-plot. TR-FRET data in this figure are presented as means±s.d. (n=3). FIG. 22G, FIG. 22K, FIG. 22L, and FIG. 22M as in FIG. 22F, but for dBET70, dBET6, dBET and dBET55, respectively.

FIG. 23A-FIG. 23D is a series of schematics and graphs showing the experimental validation of DDB1-CRBN-dBET57-BRD4_{BD1} structure. Specifically, FIG. 23A-FIG. 23D show further validation of dBET57 binding mode with TR-FRET assays. FIG. 23A is a cartoon representation of DDB1-CRBN-dBET57-BRD4_{BD1} complex with the 2F_{O}-F_{C} map contoured at 1.5 σ. Domains are colored as DDB1-BPA (red), DDB1-BPC (orange), DDB1-CTD (grey), CRBN-NTD (blue), CRBN-HBD (cyan), CRBN-CTD (green), and BRD4_{BD1} (magenta). CRBN was found in a not-previously-observed conformation, in which the thalidomide binding CRBN-CTD domain translates and rotates away from the CRBN-HBD and CRBN-NTD domains. This results in an open conformation that exposes large areas of CRBN that are typically buried. The high salt crystallization condition could be a driver of this structural rearrangement, and together with crystal contacts induce this conformation. However, it cannot be excluded that this conformational dynamic is an intrinsic feature of CRBN to accommodate a variety of substrates and future studies are necessary to address this. Based on the compatibility of the observed BRD4_{BD1} binding conformation with the open and closed CRBN conformations, for the interpretation of the data the conformational change is negligible. FIG. 23B is a cartoon representation of DDB1-CRBN-dBET57-SeMetBRD4_{BD1} complex. Anomalous difference map contoured at 3 σ shown in orange for data collected at the Se peak showing the position of the Se atoms and Zn. FIG. 23C is a schematic showing an F_{O}-F_{C} map of native DDB1-CRBN-dBET57-BRD4_{BD1} contoured at 3.0 σ and shown in green, carved around the JQ1 and thalidomide sites. Positive difference density is observed for the Thalidomide (Thal) and JQ1 binding sites. FIG. 23D is a graph showing TR-FRET, dBET6 or dBET57 degrader titrated to DDB1ΔB-CRBN_{SPYCATCHER-BODIPY}, Terbium-Streptavidin and wild type or mutants of BRD4_{BD1-biotin}. The peak height of the dose response curve for three independent replicates was quantified and is depicted as dot-plot. TR-FRET data in this figure are independent replicates presented as means±s.d. (n=3).

FIG. 24A-FIG. 24L is a series of graphs showing selective degradation of BRD4. Specifically, FIG. 24A-FIG. 24L show how family wide protein sequence alignment is used to highlight protein hotspots. Poses where these hotspots are present in the E3 ligase-target/protein interface (e.g., FIG. 24H—Q84) can be selectively targeted with heterobifunctional molecules and can result in family wide selective complex formation and resulting degradation. FIG. 24A is a graph showing the quantitative assessment of cellular degradation using EGFP/mCherry reporter assay. Cells stably expressing BRD4_{BD1}-EGFP (or constructs harbouring BRD2_{BD1}, BRD2_{BD2}, BRD3_{BD1}, BRD3_{BD2}, BRD4_{BD2}) and mCherry were treated with increasing concentrations of ZXH-2-147 and the EGFP and mCherry signals followed using flow cytometry analysis. FIG. 24B is the same as in FIG. 24A, but for ZXH-2-184. FIG. 24C is the same as FIG. 24A, but for ZXH-3-27. Data in a-c are singlicate experiments (n=1). FIG. 24D is a graph showing TR-FRET, dBET degrader titrated to DDB1ΔB-CRBN_{SPYCATCHER-BODIPY}, Terbium-Streptavidin and wild type or mutants of BRD4_{BD1-biotin}. The peak height of the dose response curve for three independent replicates was quantified and is depicted as dot-plot. TR-FRET data in this figure are presented as means±s.d. (n=3). FIG. 24E, FIG. 24F, FIG. 24G, FIG. 24H, FIG. 24I, FIG. 24J is as in FIG. 24D, but for dBET6, dBET23, dBET55, dBET57, dBET70 and ZXH-3-26 respectively. FIG. 24K is a cartoon representation of docking pose from cluster 19 (see, FIG. 21A-FIG. 21C) serving as a rationale for design of ZXH-3-26. BRD4_{BD1} shown in green and CRBN in blue. Highlighted residues of BRD4 different between BRD2/3. Residue Q84 (R in BRD2, Y in BRD3) highlighted in orange. FIG. 24L is a sequence alignment of first bromodomain of BRD2, BRD3, BRD4 and BRDT. Highlighted residues of BRD4 different between BRD2/3. Residue Q84 (R in BRD2, Y in BRD3) highlighted with an arrow. (SEQ ID Nos: 11-14 in order of appearance.)

FIG. 25 is a series of uncropped immunoblots. Boxed areas correspond to image regions represented in the indicated main text and Supplementary figures. Western blots have been flipped vertically to represent increasing concentrations of Compound. SDS-PAGE gel images for representative preparations of DDBΔB-CRBN, SeMet-BRD4_{BD1}, biotinylated BRD4_{BD1} and biotinylated BRD4_{BD2} are shown.

FIG. 26 is a schematic showing a graphical overview of some of the methods described herein. Specifically, this schematic shows that multiple suitable dimerizers can induce dimerization of two proteins A and B resulting in multiple A-dimerizer-B ternary complex poses. Finally, dimerizers can be developed to explore a specific pose, leading to selective protein dimerization and/or degradation.

While the invention comprises embodiments in many different forms, they are shown in the drawings and will herein be described in detail several specific embodiments with the understanding that the present disclosure is to be considered as an exemplification of the principles of the technology and is not intended to limit the invention to the embodiments illustrated.

### DETAILED DESCRIPTION

The present invention is based, at least in part upon the discovery and development of new and improved methods for generating heterobifunctional binders. The heterobifunctional binders can be “small molecule,” or “low molecular weight” compounds that bind, and promote interaction between, two proteins. The two proteins do not necessarily interact and/or bind in vivo. The interaction can cause a functional result such as an enzymatic activity, chemical modification, or degradation of at least one of the proteins.

In various embodiments, the methods can be used for generating small molecule heterobifunctional degraders (e.g., PROTACs or degronimids). However, the methods are also generally applicable to generating heterobifunctional binders for a first protein having a first ligand and a second protein having a second ligand. The methods can be used to create libraries of heterobifunctional binder and/or screen heterobifunctional binder (e.g., for drug discovery, development). The methods can be used to assess/predict the suitability of a target to ligand for inducing protein dimerization and/or protein degradation. The methods can be used to screen and/or interrogate protein interactions and function. A heterobifunctional binder developed using methods of the invention can be used for medical treatment, for example a cancer treatment.

Through multiple X-ray crystal structures of PROTAC bound CRL4^{CRBN}-BRD4 complexes, the Examples below demonstrate that plastic inter-protein contacts result in multiple distinct binding conformations depending on the bound PROTAC. The Examples also demonstrate that effective degradation does not require tight cooperative binding; however, distinct binding conformations are unique to ligase-substrate pairs and define selectivity. The Examples further demonstrate a computational approach to protein-protein docking and demonstrate the versatility of this approach through rational design of the first PROTAC that can discriminate between the highly homologous BET bromodomains of BRD2/3/4, leading to synthesis of a highly effective and selective BRD4 degrader.

Heterobifunctional small molecule degraders (heterobifunctional compounds or binders) that induce protein degradation through ligase-mediated ubiquitination have shown considerable promise as a new pharmacological modality. The Examples provide a detailed understanding of the molecular basis for target recruitment and selectivity, which is critically required to enable rational design of degraders. The Examples utilize comprehensive characterization of the ligand dependent CRBN/BRD4 interaction to demonstrate that binding between proteins that have not evolved to interact is unexpectedly plastic. Multiple X-ray crystal structures show that plasticity results in several distinct low energy binding conformations, which are selectively bound by ligands. The Examples demonstrate that computational protein-protein docking can reveal the underlying inter-protein contacts and inform the design of BRD4 selective degraders that can discriminate between highly homologous BET bromodomains. The Examples demonstrating that plastic inter-protein contacts confer selectivity for ligand-induced protein dimerization provide a conceptual framework for the development of high specificity heterobifunctional compounds. The Examples further provide exemplary heterobifunctional compounds that are specific for BRD4 over other BET family proteins.

Generalized methods for docking and generating heterobifunctional binders are provided herein.

In various aspects, the invention provides a method for generating a heterobifunctional binder for a first protein and a second protein. The method comprises (a) generating a first set of poses by docking a first protein structure and a second protein structure in silico: (b) generating a set of feasible poses by (i) selecting a subset of the first set poses by scoring and (ii) structurally clustering the subset in silico; (c) selecting a preferred pose from the set of feasible poses based upon the relative position and orientation of the first protein structure and the second protein structure; (d) designing a covalent linker between a first ligand for the first protein and a second ligand for the second protein in the preferred pose; and (e) synthesizing a heterobifunctional binder comprising the first ligand, the second ligand, and the covalent linker. The first and/or second ligand can be present in step (a), or can be added a later time (e.g., docked separately during step (d)).

In various aspects, the invention provides a method for generating a heterobifunctional binder for a first protein and a second protein. The method comprises (a) generating a first set of poses by docking a first protein-first ligand pair structure and a second protein-second ligand pair structure in silico; (b) generating a set of feasible poses by (i) selecting a subset of the first set poses by scoring and (ii) structurally clustering the subset in silico: (c) selecting a preferred pose from the set of feasible poses based upon the relative position and orientation of the first protein-first ligand pair structure and the second protein-second ligand pair; (d) designing a covalent linker between the first ligand and the second ligand in the preferred pose: and (e) synthesizing a heterobifunctional binder comprising the first ligand, the second ligand, and the covalent linker.

In various aspects, the invention provides a method for generating a heterobifunctional binder for a first protein and a second protein. The method comprises (a) generating, in silico, a set of poses by docking a first protein, optionally bound to a first ligand, and a second protein, optionally bound to a second ligand, where (i) a score is calculated based on energy of interactions between the first protein and the second protein for each of the poses; and (ii) a spatial relationship between the first protein and the second proteins is quantified for each of the poses, (b) generating a subset of poses by selecting one or more poses from the set of poses based on the scores of the poses, (c) identifying a candidate pose from the subset of poses based on the spatial relationship between the two proteins, (d) designing a linker between the first ligand and the second ligand that accommodates the candidate pose; and (e) synthesizing or having synthesized the heterobifunctional binder having the first ligand, the second ligand, and the linker.

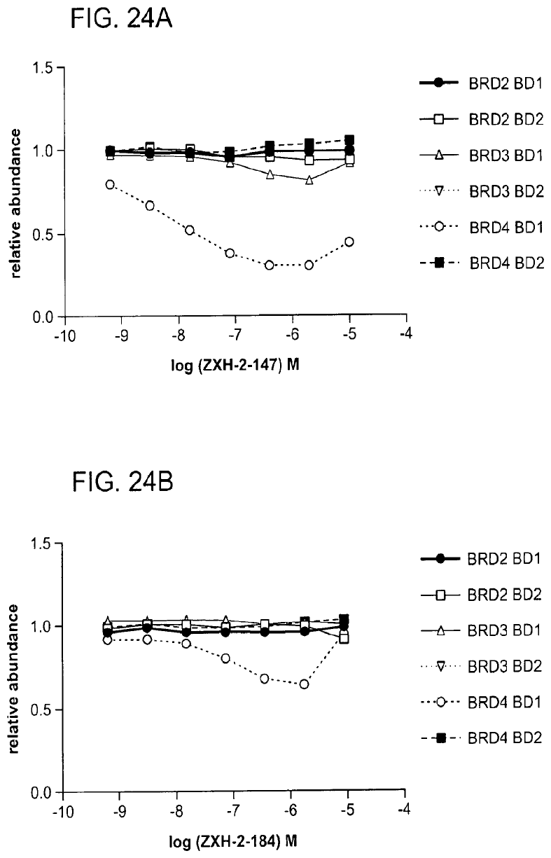

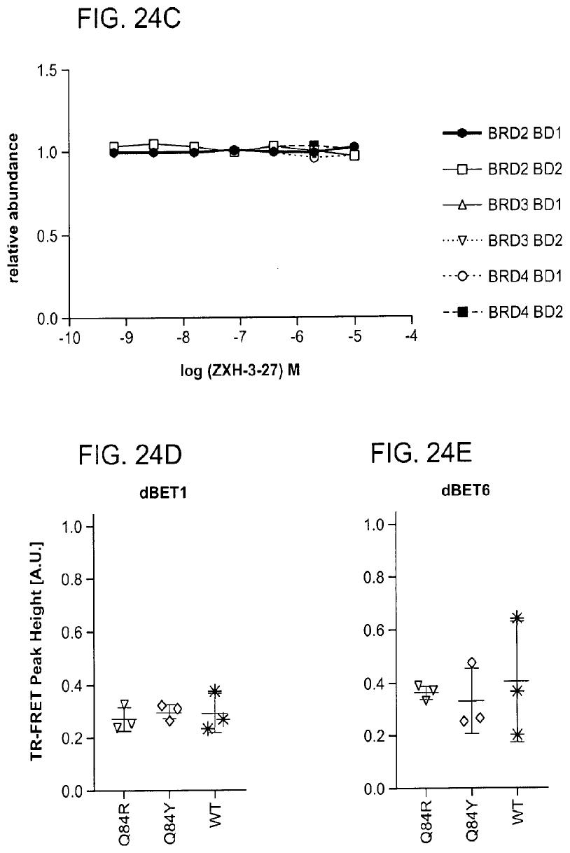

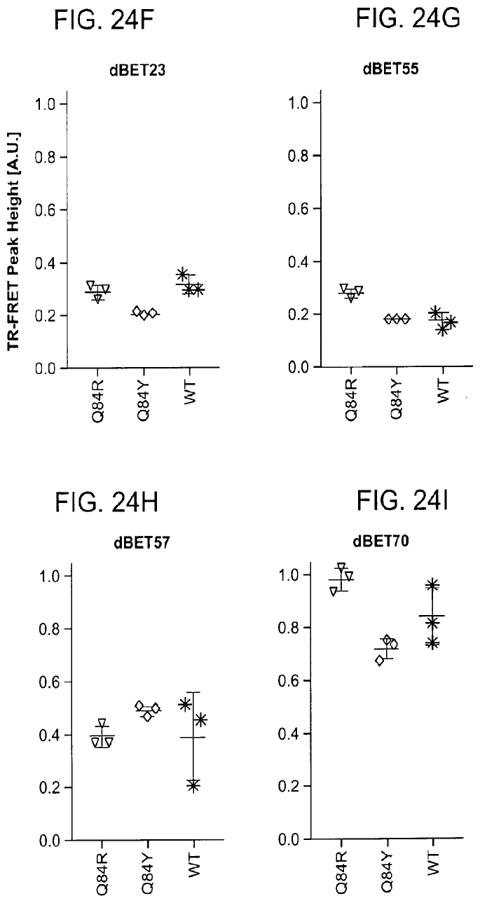

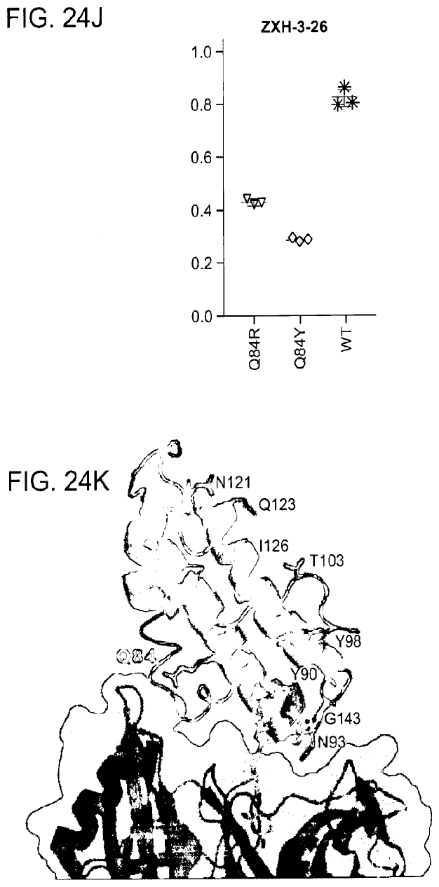

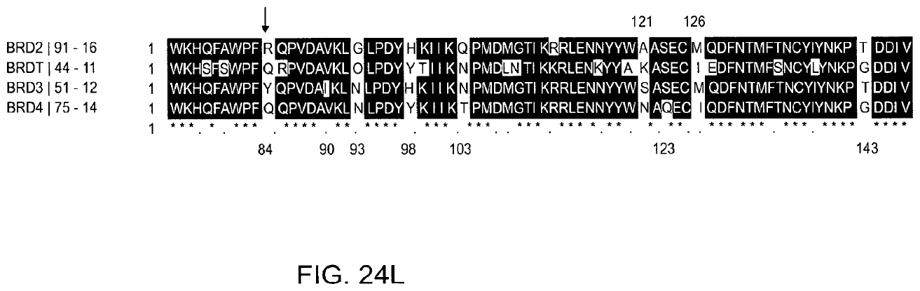

Design of selective degraders is prepared as follows. Structures (or homology models) of related (e.g., isoforms, homologs, potential-off targets) proteins are structurally aligned to their docked pose. Next, diversity hotspots are defined as locations of the protein sequence/structure with sequence diversity (such as, but not limited to, point mutations, as in FIG. 24K and FIG. 24I, Q84 in BRD4_{BD1} is R in BRD2_{BD1}, Y in BRD3_{BD1}). Then, poses are identified for which diversity hotspots present themselves in the protein-protein interface (as exemplified by FIG. 24K, Q84 as in BRD4_{BD1}). Hotspots present in the interface will likely disturb it, and potentially destabilize it, and resulting poses will favor certain mutations, translating to selective dimerization. Multiple docked poses may result in distinct interface hotspots, which can be explored to direct dimerization selectivity to the target. Design of non-selective degraders is achieved in the same method by in turn focusing on poses that have no hotspots in the protein-protein interface.

As will be understood by those skilled in the art, the aspect above can be combined with any one or more of the features below.

In various embodiments, the invention further comprises experimentally measuring binding of the first protein, the second protein, and the heterobifunctional binder.

In some embodiments, a binder is selected based upon the binding specificity or affinity being above a predetermined threshold (e.g., compared to a reference heterobifunctional binder or a library of heterobifunctional binders or a heterobifunctional binder having a different linker).

In various embodiments, the invention further comprises experimentally measuring a functional result of binding the first protein, the second protein, and the heterobifunctional binder. The functional result comprises an enzymatic activity, chemical modification, or degradation of the first or second protein.

In some embodiments, a binder is selected based upon the functional result being above a predetermined threshold (e.g., compared to a reference heterobifunctional binder or a library of heterobifunctional binders or a heterobifunctional binder having a different linker).

In various embodiments, the invention further comprises synthesizing a library of heterobifunctional binders. A library can include on the order of 10, 10², 10³, 10⁴, 10⁵, or 10⁶ binders.

In various embodiments, the invention further comprises experimentally screening the library of heterobifunctional binders.

In various embodiments, the step of synthesizing, measuring, or screening can include synthesizing, measuring, or screening carried out by a third party such as a collaborator or contractor. The step of synthesizing, measuring, or screening can include instructing/directing a third party to carry out the step of synthesizing, measuring, or screening.

In various embodiments, the first and second proteins do not naturally bind each other in vivo. For example, the proteins may not be parts of a multimeric protein, protein complex, or normally interacting protein pair (e.g., the binding having been subjected to evolutionary selection).

In various embodiments, the first protein or the second protein is a ubiquitin ligase. The ubiquitin ligase can be an E3 ubiquitin ligase or a component of the E3 ubiquitin ligase. The E3 ubiquitin ligase can be CRL4^{CRBN}, CRL4^{DCAF15}, CRL3^{KEAP1} or CRL2^{VHL}. The component of the E3 ubiquitin ligase can be CRBN, DCAF15, KEAP1, or VHL.

In various embodiments, the first protein or the second protein is an E2 ubiquitin conjugating enzyme.

In various embodiments, the first protein or the second protein is a Von Hippel-Lindau tumor suppressor protein (VHL).

In various embodiments, the first protein or the second protein is a subunit of a proteasome.

In various embodiments, the first ligand or the second ligand is a ubiquitin ligase ligand.

In various embodiments, the first ligand or the second ligand is an E3 ubiquitin ligase ligand.

In various embodiments, the first ligand or the second ligand is a ligand for a component of an E3 ubiquitin ligase.

In various embodiments, the first ligand or the second ligand is thalidomide, lenalidomide, pomalidomide, or an analog or derivative thereof.

In various embodiments, the first ligand or the second ligand is a E2 ubiquitin conjugating enzyme ligand.

In various embodiments, the first ligand or the second ligand is a Von Hippel-Lindau tumor suppressor protein (VHL) ligand.

In various embodiments, the first ligand or the second ligand is a proteasome subunit ligand.

In various embodiments, step (d) further comprises calculating a shortest path or shortest distance between the first and second ligands. The shortest path can be calculated between a centroid and/or a predetermined atom of each of the first and second ligands.

Shortest distance can be calculated as minimum Euclidean distance between a centroid and/or a predetermined atom of each of the first and second ligands.

In various embodiments, the invention further comprises fitting a chemical structure to the shortest path, thereby designing the covalent linker.

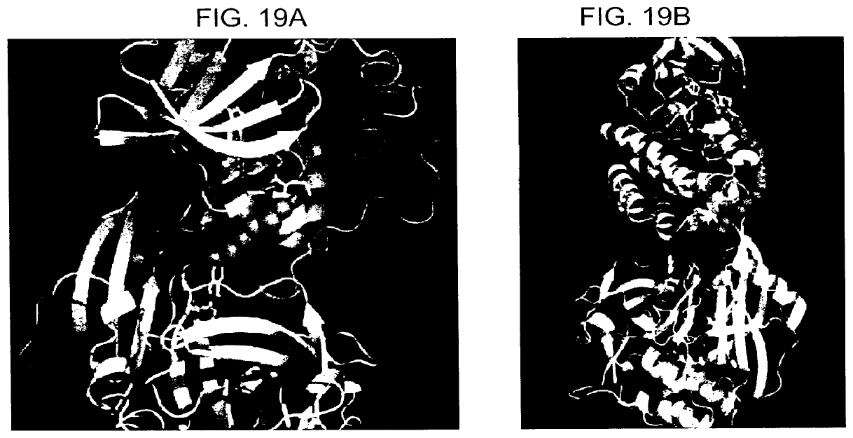

FIG. 19A-FIG. 19H show an example linker development algorithm. FIG. 19A shows an example shortest path calculation. FIG. 19B shows an example long path calculation.

In various embodiments, the method can include providing a histogram of linker lengths, providing histogram of most common exit atoms as spheres with size as variable, and/or output of docking as cloud of centroids and as sphere of orientations.

An example linker design algorithm can include one or more of the following steps: (1) for each docked pose (protein B with ligand docked to protein A with ligand) create a 3D grid of points of the dimension of the docked pose, and represent them as a graph with adjacency matrix describing point to point connectivity, all points connected to each immediate neighbor point, (2) Load the x,y,z atom coordinates of the docked pose and interpolate them on the 3D graph, load the start_path atom coordinates on ligand A and end_path atom coordinates on ligand B, (3) remove the interpolated points from the 3D graph, and (4) calculate the shortest path with Dijkstra algorithm between start_path and end_path.

In various embodiments, the covalent linker is an alkyl or PEG linker.

In various embodiments, the first protein-first ligand pair structure and/or the second protein-second ligand pair structure can be experimentally or computationally derived.

In various embodiments, the first set of poses can include about 10,000 to 50,000 poses, about 50,000 to 100,000 poses, or about 25,000 to 250,000 poses.

In various embodiments, the subset of the first set poses can include about 100, 200, 300, 400, 500, 600, 700, 800, 900, 1,000, or 10,000 poses. The first set of poses can include about 100-1,000 or 100-10,000 or 1,000-10,000 poses.

In some embodiments, a heterobifunctional binder or a library of heterobifunctional binders is a molecule or a set of molecules selected from the genera described in US Patent Application Publication No. 2016/0176916 (U.S. Ser. No. 14/707,930), for example, as provided in Formula X, I or II.

In various embodiments, the preferred pose comprises a set of preferred poses.

In various embodiments, the method comprises designing a set of heterobifunctional binders. The set of heterobifunctional binders can correspond to the set of preferred poses.

In various embodiments, step (d) further comprises docking a first ligand to the first protein and/or a second ligand to the second protein (e.g., where the first and/or second ligand is not docked in step (a) or where the first and/or second ligand is changed in step (d) or where the first and/or second ligand structure is refined in step (d)).

In various embodiments, the method further comprises assessing/predicting the suitability of a target to ligand for inducing protein dimerization and/or protein degradation.

In various embodiments, the method further comprises assessing/predicting the suitability of a target to ligand for inducing protein dimerization and/or protein degradation. For example, this can be achieved using the principle that a target yielding long linker paths will probably result in a degrader with low cellular-permeability (or any other parameter known and used in structure activity relationships) and therefore low activity.

In various embodiments, the heterobifunctional binder causes degradation of the first protein with a higher specificity than the binding specificity of the first ligand for the first protein.

In various embodiments, the spatial relationship between the first protein and the second protein is quantified by calculating the shortest path between a first set of solvent-exposed atoms on the first ligand and a second set of solvent-exposed atoms on the second ligand.

In various embodiments, the spatial relationship between the first protein and the second protein is quantified by calculating the shortest path between the centroid of the first ligand and the centroid of the second ligands.

In various embodiments, the heterobifunctional binder dimerizes the first protein and the second protein in a low-energy level conformation.

In various embodiments, the plurality of shortest paths calculated is compiled to generate a distance profile for the subset of poses.

In various embodiments, the distance profile of the subset of poses has a distinct cluster of poses that have similar shortest paths.

In various embodiments, the candidate pose is the lowest scoring pose of the cluster of poses.

In various embodiments, the specificity of the heterobifunctional binder for the first protein and the second protein is predicted from the distance profile for the subset of poses.

In various embodiments, relative specificity the heterobifunctional binder for two different first proteins can be predictively distinguished by comparing the distance profiles for the subset of poses for each of the two different first proteins and the second protein.

In various embodiments, the method further comprises experimentally measuring binding of the first protein, the second protein, and the heterobifunctional binder.

In various embodiments, the method further comprises experimentally measuring a functional result of binding the first protein, the second protein, and the heterobifunctional binder.

In various embodiments, the functional result comprises an enzymatic activity, chemical modification, or degradation of the first or second protein.

In various embodiments, the method further comprises synthesizing a library of heterobifunctional binders.

In various embodiments, the method further comprising experimentally screening the library of heterobifunctional binders.

In various embodiments, the first and second proteins do not naturally bind each other in vivo.

In various embodiments, the first protein or the second protein is a ubiquitin ligase.

In various embodiments, the ubiquitin ligase is an E3 ubiquitin ligase.

In various embodiments, the ubiquitin ligase is a component of an E3 ubiquitin ligase.

In various embodiments, the E3 ubiquitin ligase is CRL4^{CRBN}, CRL4^{DCAF15}, CRL3^{KEAP1} or CRL2^{VHL}.

In various embodiments, the component of the E3 ubiquitin ligase is CRBN, DCAF15, KEAP1, or VHL.

In various embodiments, the first protein or the second protein is an E2 ubiquitin conjugating enzyme.

In various embodiments, the first protein or the second protein is CRL2^{VHL}.

In various embodiments, the first protein or the second protein is a subunit of a proteasome.

In various embodiments, the first ligand or the second ligand is a ubiquitin ligase ligand.

In various embodiments, the first ligand or the second ligand is an E3 ubiquitin ligase ligand.

In various embodiments, the first ligand or the second ligand is a ligand for a component of an E3 ubiquitin ligase ligand.

In various embodiments, the first ligand or the second ligand is thalidomide, lenalidomide, pomalidomide, or an analogue or derivative thereof.

In various embodiments, the first ligand or the second ligand is a E2 ubiquitin conjugating enzyme ligand.

In various embodiments, the first ligand or the second ligand is a Von Hippel-Lindau tumor suppressor protein (VHL) ligand.

In various embodiments, the first ligand or the second ligand is a proteasome subunit ligand.

In various embodiments, the step of designing the linker further comprises fitting a chemical structure to the shortest path of the candidate pose, thereby designing the linker.

### Discussion of Examples

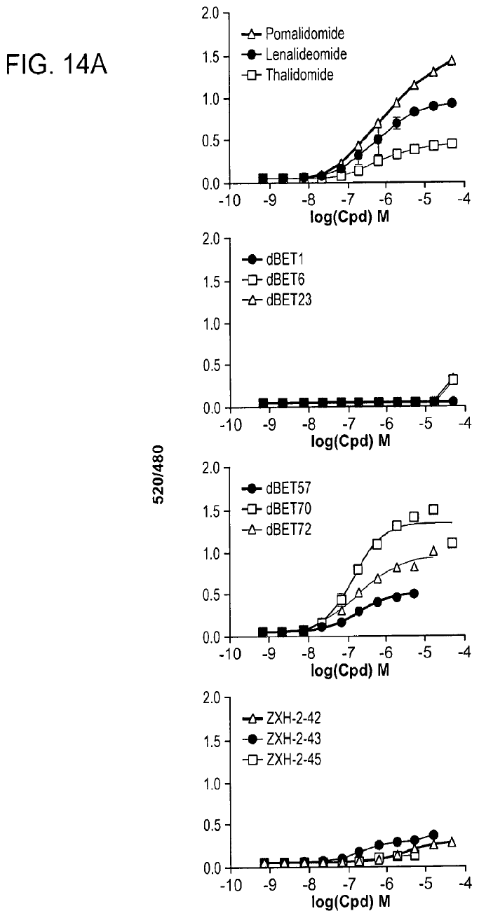

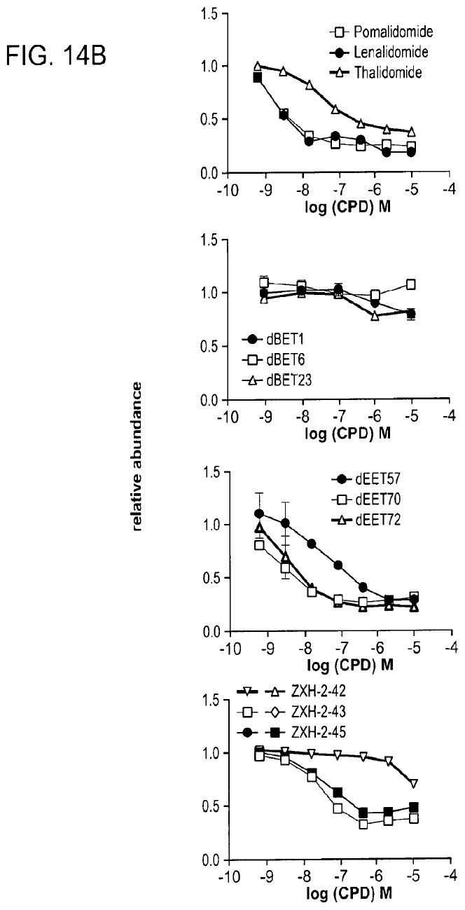

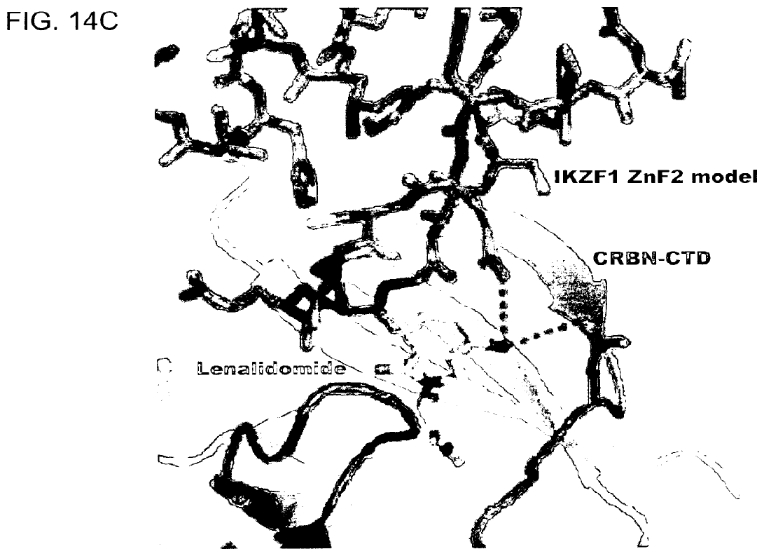

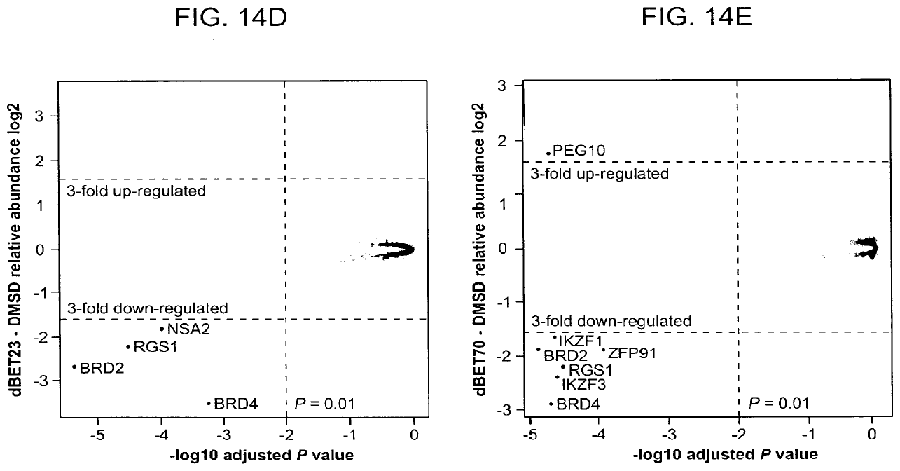

An integrated approach combining structural, biochemical, and cellular data was used to establish the molecular basis of PROTAC-mediated neo-substrate recruitment to the CRL4^{CRBN} E3 ubiquitin ligase. The Examples herein show that inter-protein contacts, while contributing relatively little binding affinity to the interaction, can be drivers of selectivity, and that highly effective degraders (e.g. the low nanomolar cellular activity of dBET6 or dBET70) can be achieved in absence of tight binding or positive cooperativity. Through multiple X-ray crystal structures together with comprehensive biochemical, cellular, and computational characterization, the Examples demonstrate that binding between ligase and substrate is surprisingly plastic and thus adapt distinct conformations depending on linker length and position. The Examples also demonstrate that exploiting such ‘local’ energy/entropy minima underlies selectivity as seen for dBET57. The Examples further demonstrate that in silico protein docking can be used to reveal low energy binding modes and can guide development of heterobifunctional degraders that can discriminate between the highly homologous BET bromodomains, such as ZXH-03-26. The Examples herein further demonstrate that biochemical properties translate to cellular activity with respect to BRD4 on-target and IKZF1 off-target degradation and that the IKZF1 degradation can be tuned by IMiD linker composition (FIGS. 14A-E).

The Examples herein demonstrate that the same two proteins can bind in different overall conformations, which results in distinct surface patches on the ligase and target to interact. This plasticity underlies the principle of selectivity. PROTACs therefore appear to exploit natural and widely occurring non-specific interactions by increasing the local concentration of the two protein binding partners. Non-specific interactions are widespread and thought to occur between any two proteins with affinities >10 mM (Kuriyan and Eisenberg 2007). However, these interaction surfaces are not random as they require a certain degree of surface complementarity to avoid unfavourable contacts such as opposing charged surfaces. The constraints of relatively short linkers result in only few accessible inter-protein contact conformations. In theory, rationally designed linkers restricted to a specific binding mode unique to a ligase/substrate pair should be sufficient to drive selectivity since such a restricted conformation is unlikely to occur in a close orthologue. The Examples herein show that such can be achieved in practice with the compound ZXH-03-26.

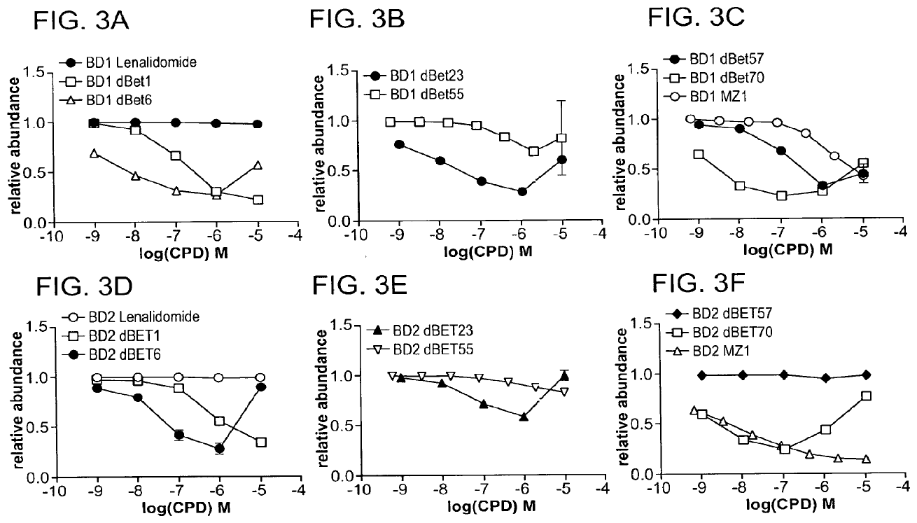

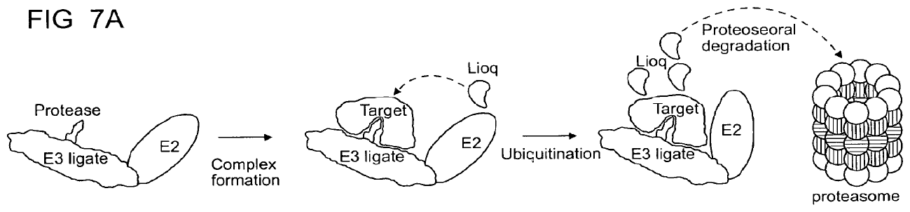

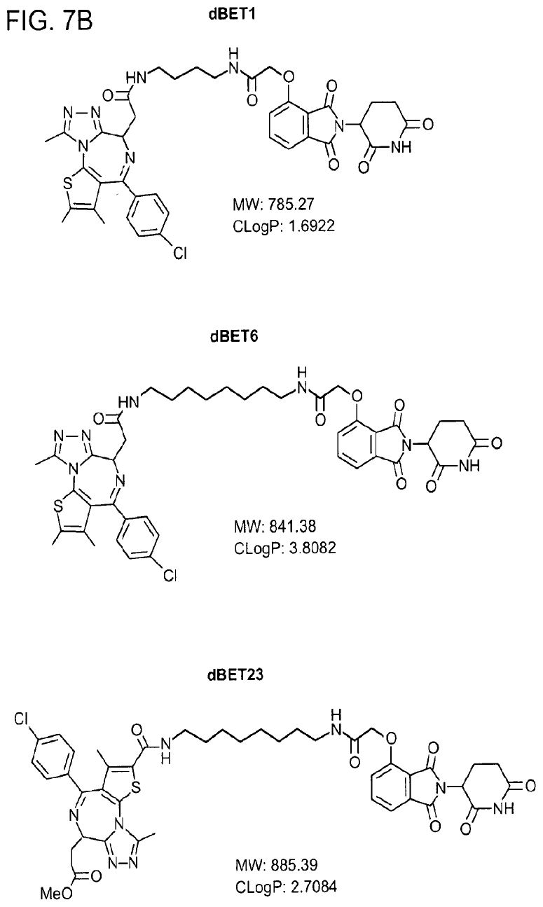

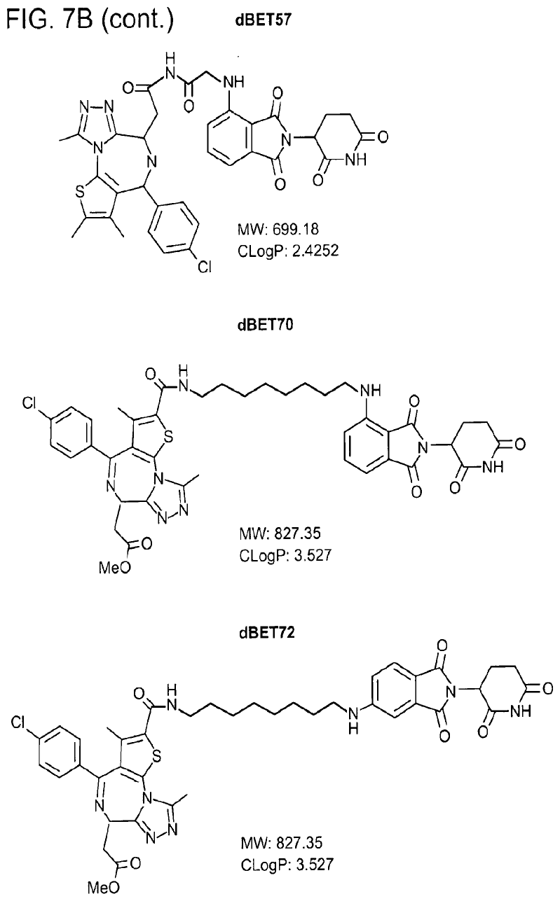

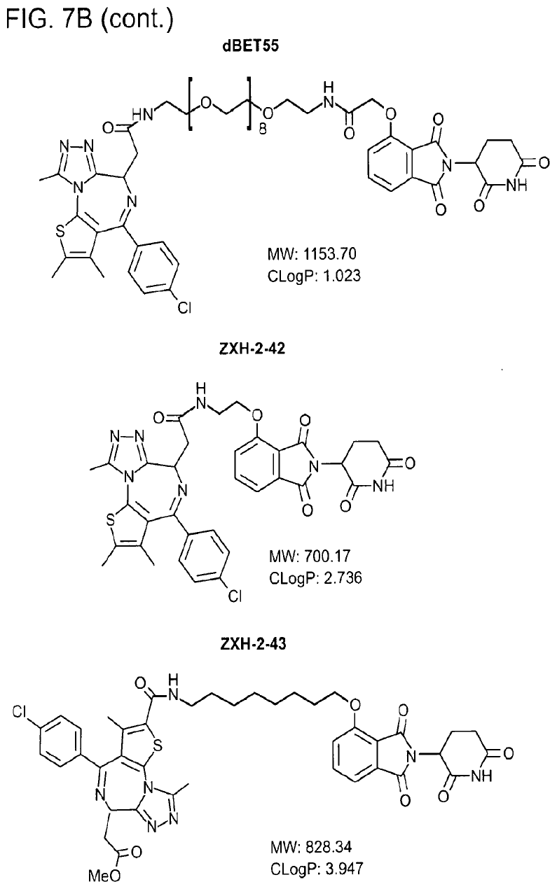

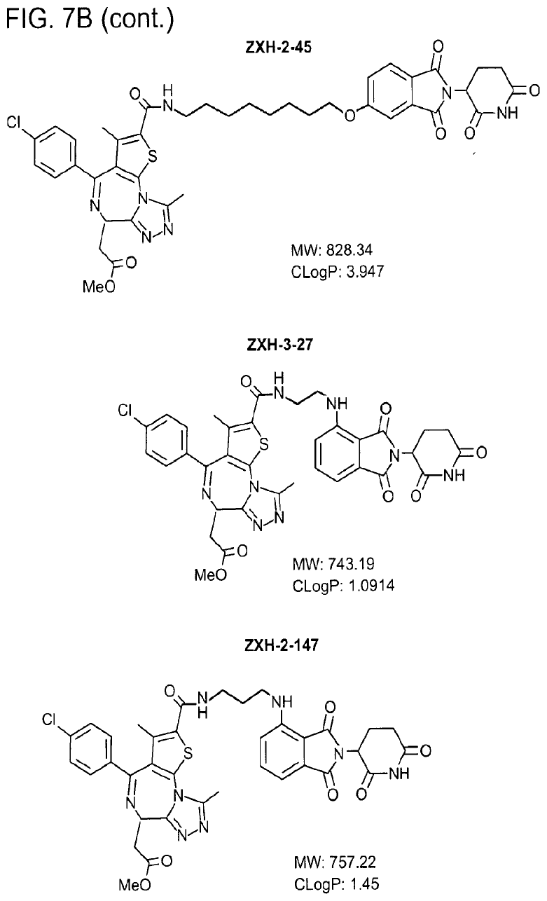

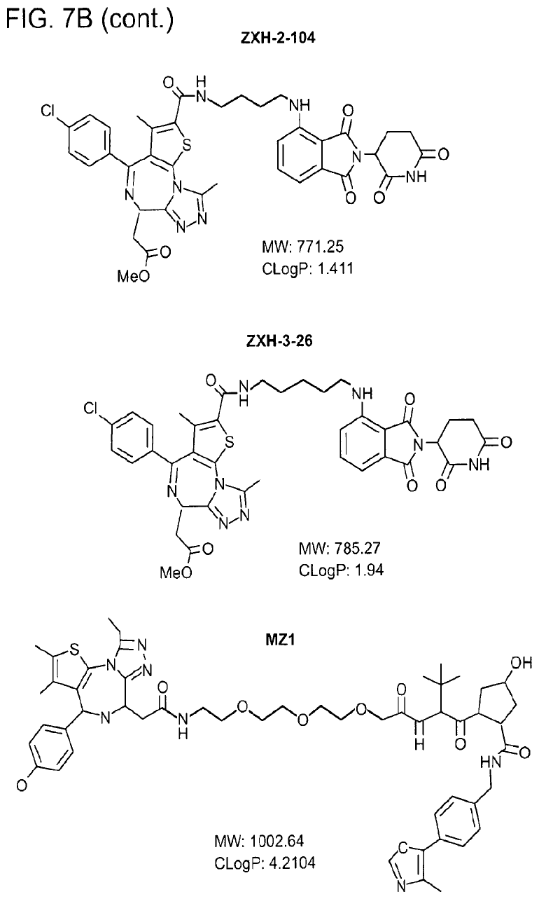

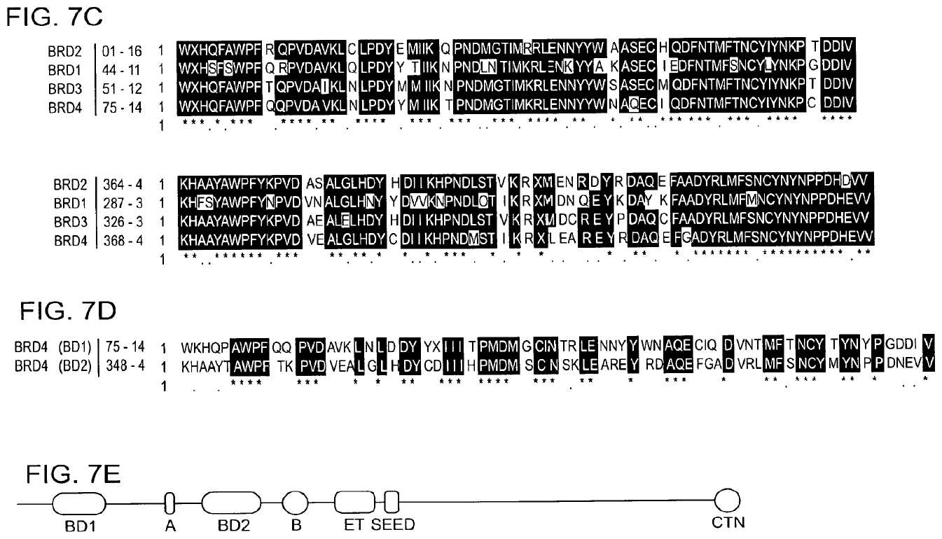

The absence of positive cooperativity and the existence of multiple distinct binding conformations carries further important implications. The unnecessity for high affinity ligase-substrate interactions implies that a wide variety of E3 ligases can be explored to achieve desirable properties such as tissue specificity. The Examples herein demonstrate with dBET57 and ZXH-03-26 that effective PROTACs can be designed to harbour relatively short linkers, which results in favourable and more ‘drug-like’ overall properties (FIG. 7B). The Examples herein demonstrate that such short linker compounds exhibit high selectivity since the number of accessible binding conformations is reduced. Selectivity can also be further explored using different E3-moeities, as seen for CRBN- and VHL-targeting PROTACs (FIGS. 3A-C). The Examples herein demonstrate that computational modelling can provide an elegant surrogate, which depends only on a known structure for the individual components (ligase and target), and has the potential to enable initial predictions of possible linker length and trajectory to guide medicinal chemistry.

With ZXH-03-26, ZXH-2-184, ZXH-2-147, and ZXH-3-82, the Examples herein provides working examples of heterobifunctional compounds that selectively targets BRD4 for degradation and spares BRD2 and BRD3, which also represents the first small molecule to allow pharmacologic targeting of BRD4 without significant inhibition/degradation of BRD2/3. This has implications for future developments since efficacy of BRD4 inhibition has been established for a variety of malignancies (Zuber, Shi et al. 2011, Chau, Hurwitz et al. 2016), while on-target toxicity has been observed in pre-clinical and clinical studies (Stathis, Zucca et al. 2016). It is conceivable that selective degradation of BRD4 will retain efficacy, while significantly reducing on-target toxicity in NUT midline carcinomas, which depend on the BRD4-NUT fusion protein. Such selective targeting of an oncogenic fusion protein has been shown as effective treatment strategy in the case of BCR-ABL and Gleevec (Buchdunger, Cioffi et al. 2000). ZXH-03-26, ZXH-2-184, ZXH-2-147, and ZXH-3-82 present examples of heterobifunctional compounds that can selectively degrade the BRD4-NUT oncogenic fusion protein.

The following examples are illustrative and not restrictive. Many variations of the technology will become apparent to those of skill in the art upon review of this disclosure. The scope of the technology should, therefore, be determined not with reference to the examples, but instead should be determined with reference to the appended claims along with their full scope of equivalents.

### EXAMPLES

The following examples present a comprehensive structural, biochemical and cellular analysis of dBET degrader-mediated BRD4 recruitment to CRL4^{CRBN}. The examples demonstrate that the ligase-degrader-substrate binding mode is unexpectedly plastic, and that this plasticity results in multiple low energy binding conformations that can be exploited to achieve target specificity. The examples show that computational docking can reveal these energetically favorable binding modes and help to rationalize degrader specificity. These fundamental principles of ligand induced dimerization apply to systems beyond targeted protein degradation such as allosteric regulators or protein-dimerization.

General Comments on Heterobifunctional Degrader Design

The present Examples demonstrate the occurrence and putative role of inter-protein contacts in either strengthening a substrate-ligase complex or conferring target selectivity. However, as shown for CRBN-BRD4_{BD1}, it is likely that more than one possible binding mode exists. Here, it is shown that distinct binding modes can be exploited and can result in selective molecules.

The present Examples also provides working examples of bifunctional binders designed by the present invention.

### Example 1: BRD4 Contains Two Bromodomains

Since small changes to the PROTAC can result in dramatically altered cell permeability or solubility, the Examples below devised a synthetic system based on the recruitment of isolated BRD4 bromodomains to CRL4^{CRBN}. Like other members of the BET family, BRD4 contains two bromodomains: bromodomain 1 (aa 75-147 and referred to as BRD4_{BD1}) and BRD4_{BD2} (aa 368-440), and sequence conservation between the two is limited (FIGS. 7C-E). These distinct domains bind the JQ1 based target-moiety with equal affinities (Filippakopoulos, Qi et al. 2010), hence establish a model system to understand how amino acid sequence and thereby protein surface properties influence protein dimerization. The Examples below utilized a series of compounds synthesized to bind CRBN and the bromodomains of BRD4 (referred to as dBETs, see FIG. 7B) (Winter, Buckley et al. 2015), dBET molecules comprise the E3-moiety thalidomide to bind to CRL4^{CRBN}, a flexible linker of variable length and composition, and a target-moiety, JQ1, that binds to BRD4_{BD1} and BRD4_{BD2} with equal affinities (Filippakopoulos, Qi et al. 2010).

### Example 2: Crystal Structure of a DDB1ΔB-CRBN-dBET23-BRD4_{BD1} Complex

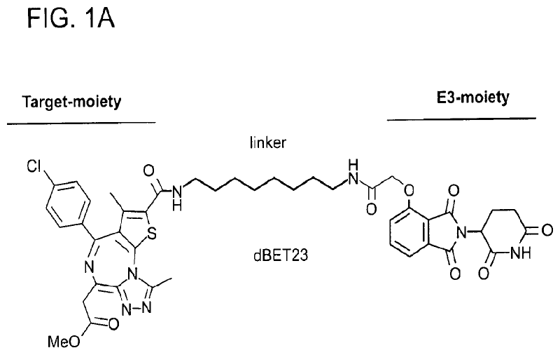

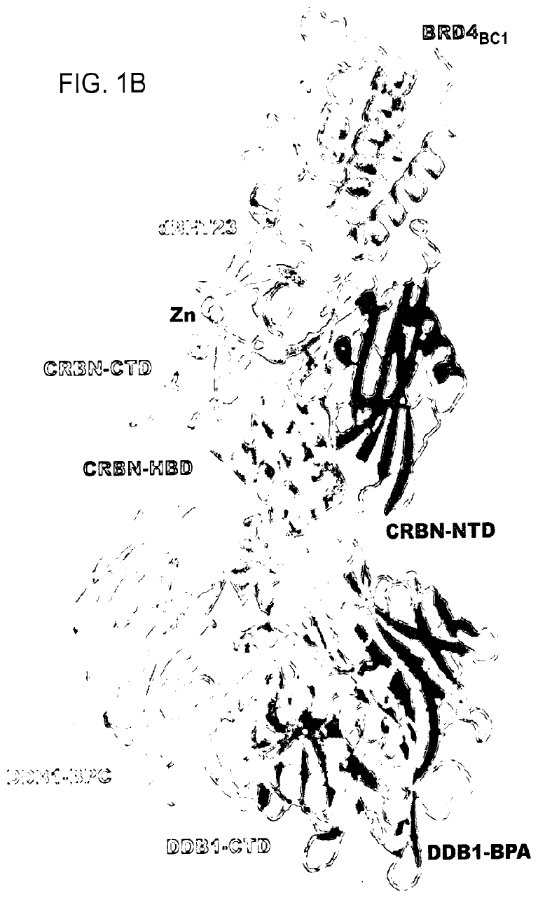

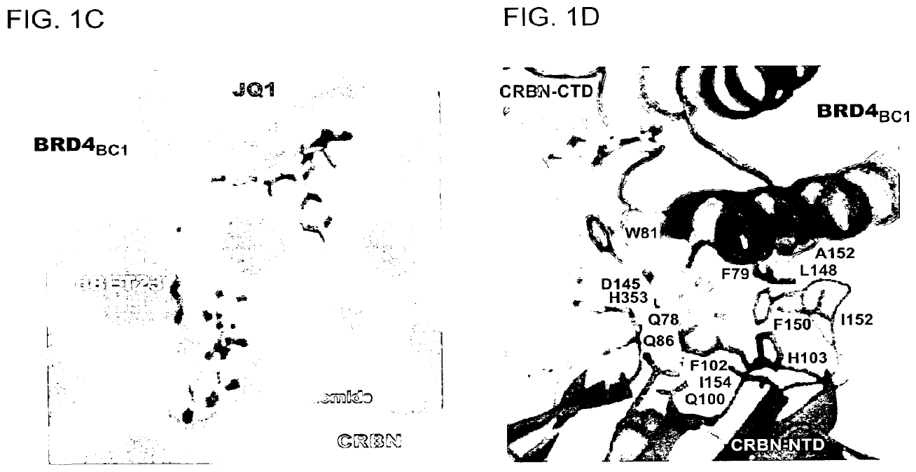

To determine the structural basis of BRD4 recruitment to CRBN, DDB1ΔB-CRBN, and BRD4_{BD1} complexes bound to different dBET molecules were reconstituted. Initial crystals were obtained for the ˜165 kDa hsDDB1ΔB-hsCRBN-dBET23-hsBRD4_{BD1} (dBET23 comprises an 8-carbon linker to bridge the oxy-acetamide of pomalidomide to the thiophene group of JQ1) complex and its structure was determined to 3.5 Å resolution (FIG. 1B) by molecular replacement using a DDB1ΔB-CRBN model (PDB: 5fqd, see Table 1). The DDB1 β-propeller domains A and C (BPA and BPC) bind CRBN but do not contribute contacts to BRD4_{BD1}. CRBN consists of three domains, the N-terminal domain (NTD), the helical-bundle domain (HBD) and the C-terminal domain (CTD), which harbours the thalidomide binding pocket (Fischer, Böhm et al. 2014). The small molecule degrader dBET23 occupies the canonical binding sites on CRBN and BRD4_{BD1} for lenalidomide and JQ1, respectively (FIG. 1C).

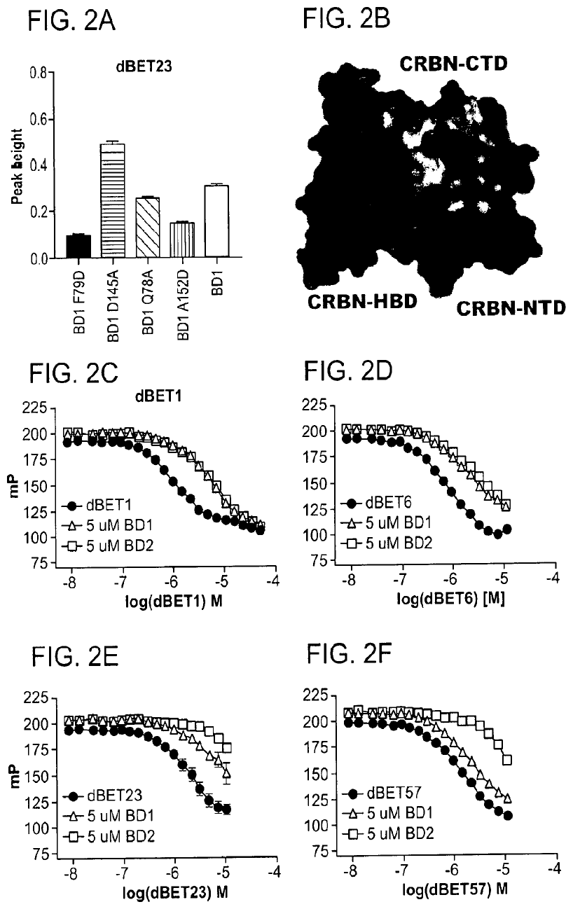

BRD4_{BD1} interacts with CRBN through contacts with the NTD domain of CRBN and with CRBN residues in direct proximity to the thalidomide-binding pocket (FIG. 1D). CRBN binds the BRD4_{BD1} αC helix (aa 145-161) and residues in the BRD4_{BD1} ZA loop (aa 76-104) (Filippakopoulos, Picaud et al. 2012). The αC helix forms hydrophobic interactions with two loops in the CRBN-NTD (aa 101-104 and as 147-154). Residues Leu148, Met149, Ala152, and Leu156 in the αC helix together with His77 and Phe79 in the ZA loop, form a hydrophobic patch that interacts with Phe102, His103, Phe150, Gly151, Ile152, and Ile154 in the CRBN-NTD. BRD4_{BD1} Gin78 forms a hydrogen bond with Gln100 in the CRBN-NTD (FIG. 1D). Consequently, mutations of the BRD4_{BD1} residues Phe79Asp, Ala152Asp, and Gln78Ala all reduce tertiary complex formation as monitored by measuring the peak-height in a TR-FRET dimerization assay (FIG. 2A). The Examples further showed that Asp145 is buried in a hydrophobic environment, and accordingly, introducing an Asp145Ala mutation strengthens the binding of BRD4_{BD1} to CRBN (FIG. 2A). The interaction between CRBN and BRD4_{BD1} consists of a total buried surface area of ˜550 Å² (FIG. 2B) (Krissinel and Henrick 2007), comparable to that observed for CRBN-Ck1α (˜600 Å²) and GSPT1 (˜600 Å²) (Matyskiela, Lu et al. 2016, Petzold, Fischer et al. 2016).

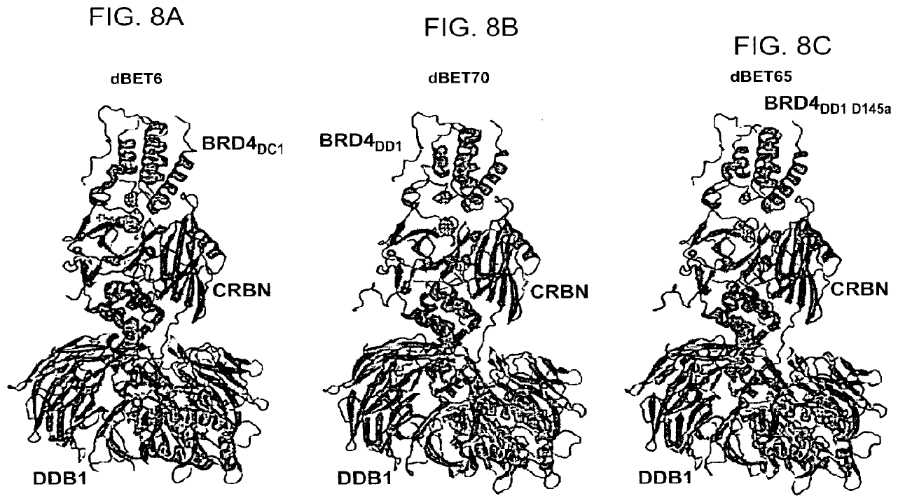

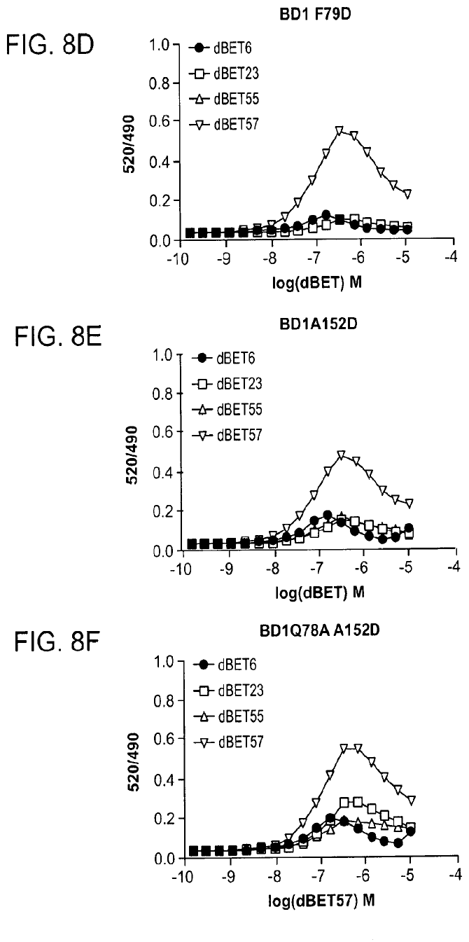

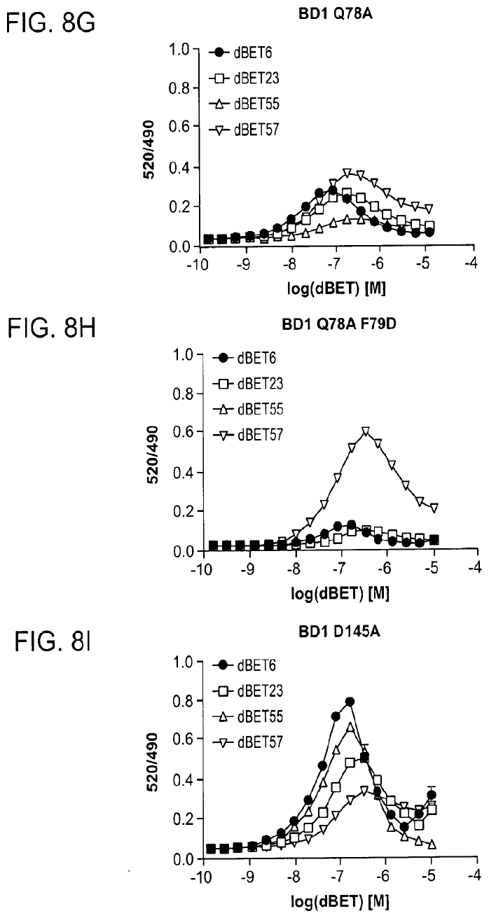

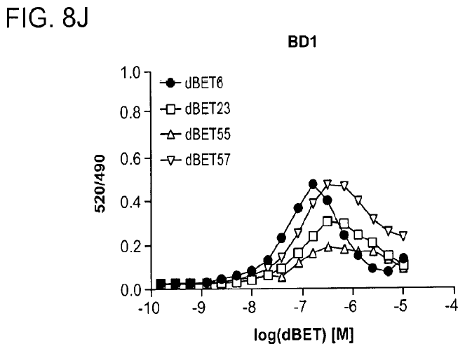

In addition to dBET23, the Examples determined crystal structure with the related molecules dBET6 (3.3 Å resolution), dBET70 (4.3 Å resolution)—both have linkers of similar length—and significantly longer dBET55 (4.0 Å resolution and crystallized with BRD4_{BD1 (D145A)}). The overall structures of these complexes are comparable to the structure obtained with dBET23 (FIGS. 8A and B) and the involvement of near identical inter-protein contacts is further confirmed by similar effects of BRD4_{BD1} interface mutations on complex formation (FIG. 8C).

### Example 3: Inter-Protein Contacts are Unique to BRD4_{BD1}

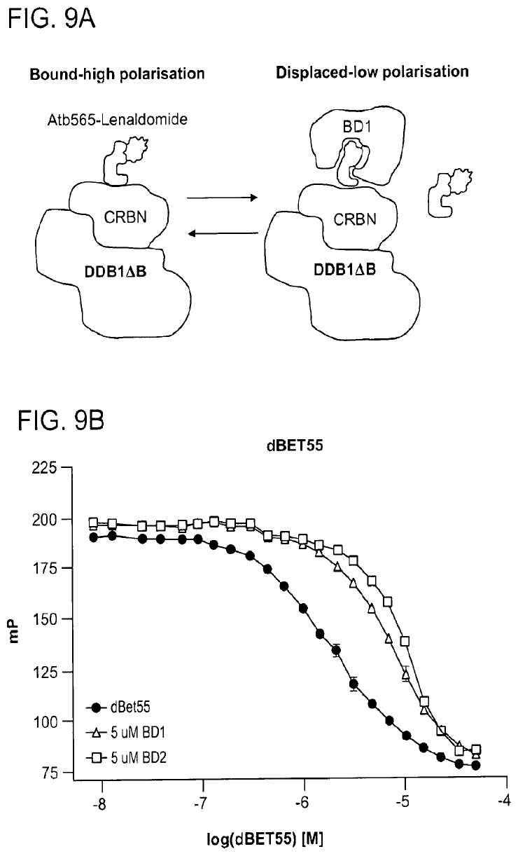

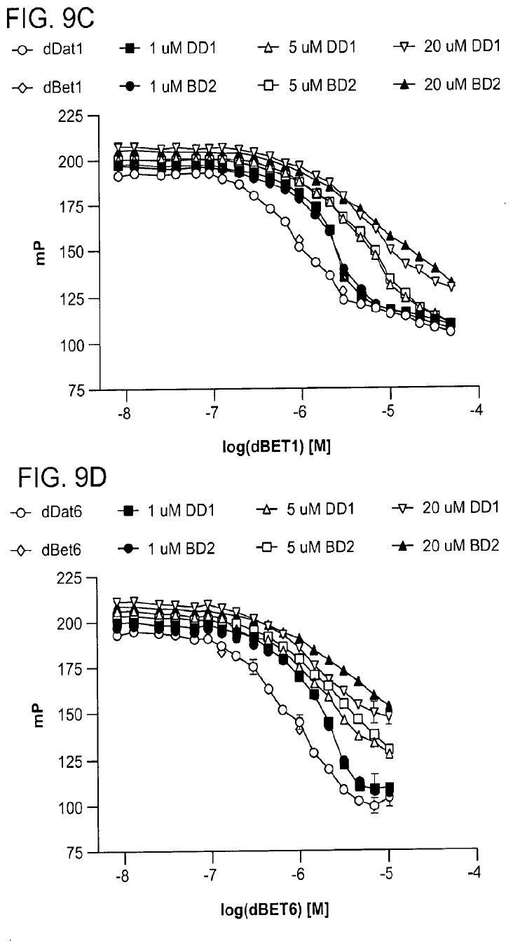

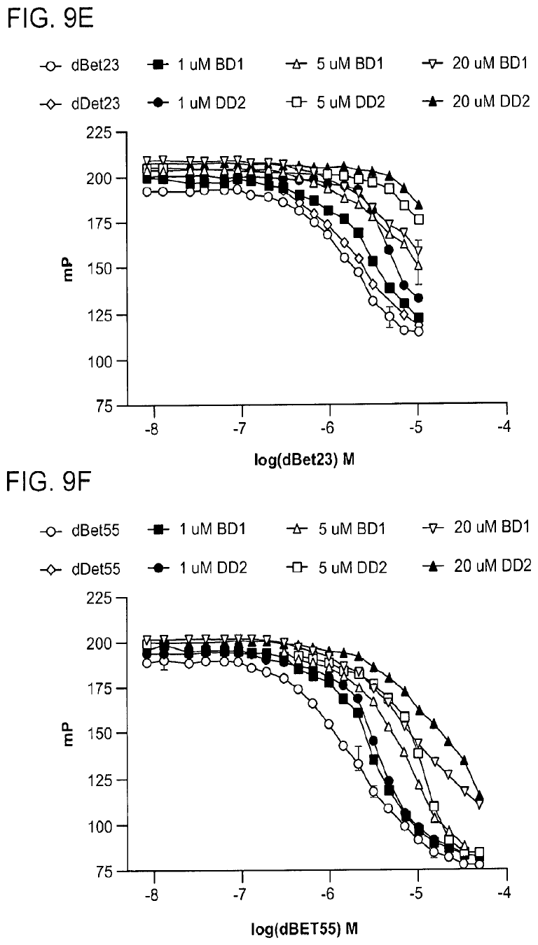

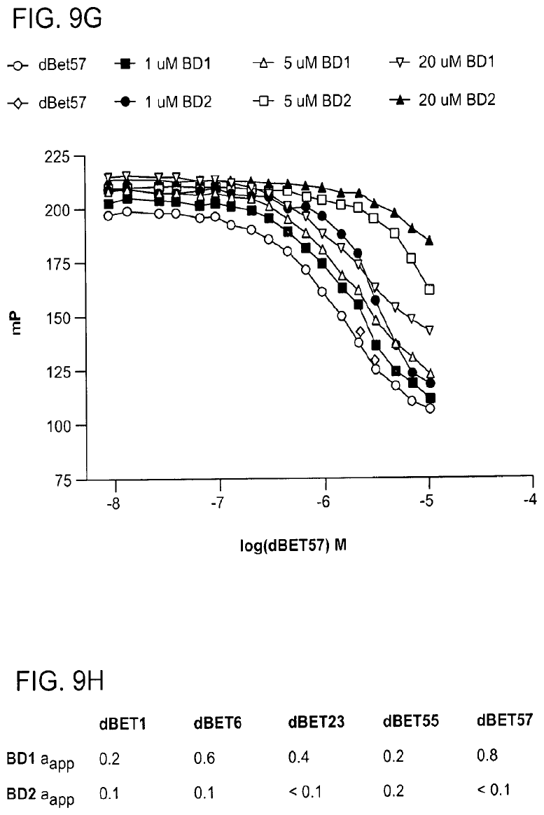

The amino acid sequences of BRD4_{BD1} to BRD4_{BD2} are 49% similar (FIG. 7D), yet none of the key residues in the αC helix or the ZA loop involved in contacts with CRBN are identical. The Examples addressed whether affinity of BRD4_{BD2} for CRBN is reduced in the presence of dBET6 or dBET23. While the determination of absolute binding affinities is difficult for a three body binding problem (Douglass, Miller et al. 2013), a qualitative measure of the relative affinities (or cooperativity of binding) can be indirectly obtained through CRBN-dBET binding assays in presence or absence of purified BRD4_{BD1} or BRD4_{BD2} protein. Using a lenalidomide-Atto565 fluorescent probe, binding of dBETs to CRBN was measured by competitive titration (FIGS. 2C-F). Next, the Examples show similar binding experiments in presence of increasing concentrations of either BRD4_{BD1} or BRD4_{BD2} to assess the cooperativity of ternary complex formation. An apparent cooperativity factor alpha was defined as αₐₚₚ=IC₅₀[binary]/IC₅₀[temary], with positive cooperativity resulting in a, >1, and negative cooperativity in αₐₚₚ<1 (see FIGS. 2C-F and FIGS. 9A-G), dBET6, exhibited an IC₅₀ of ˜0.8 μM in the absence of BRD4, which increases to an IC₅₀ of ˜1.8 μM (αₐₚₚ=0.6) in the presence of BRD4_{BD1}, and an IC₅₀ of ˜4.1 μM (αₐₚₚ=0.2) in the presence of BRD4_{BD2} (FIG. 2D and FIGS. 9A-C), indicative of negative cooperativity for both BRD4_{BD1} and BRD4_{BD2}. For dBET23 and dBET57 the difference between BRD4_{BD1} and BRD4_{BD2} is more pronounced, with αₐₚₚ=0.4 (dBET23) and αₐₚₚ=0.8 (dBET57) for BRD4_{BD1} and a, <0.1 for BRD4_{BD2} (the binding in presence of BRD4_{BD2} is too weak to quantify), indicating negative cooperativity and a preference for binding to BRD4_{BD1} (FIGS. 2E and F and FIGS. 9A-G).

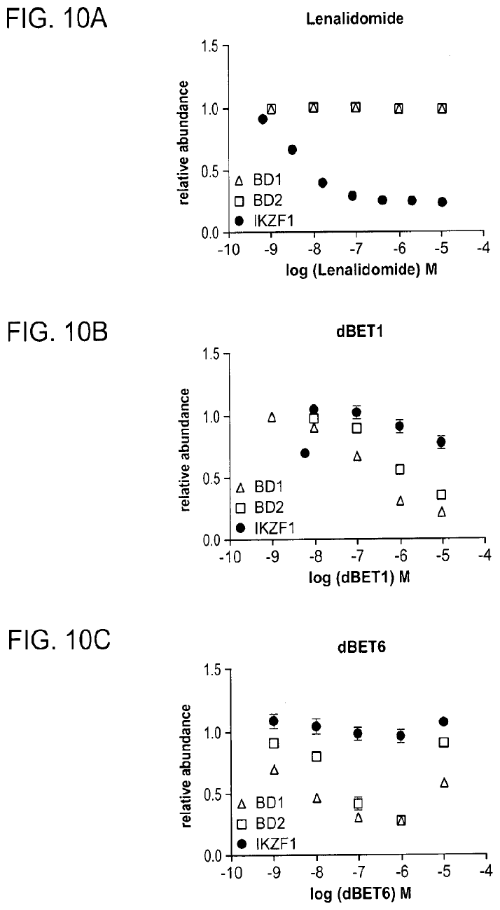

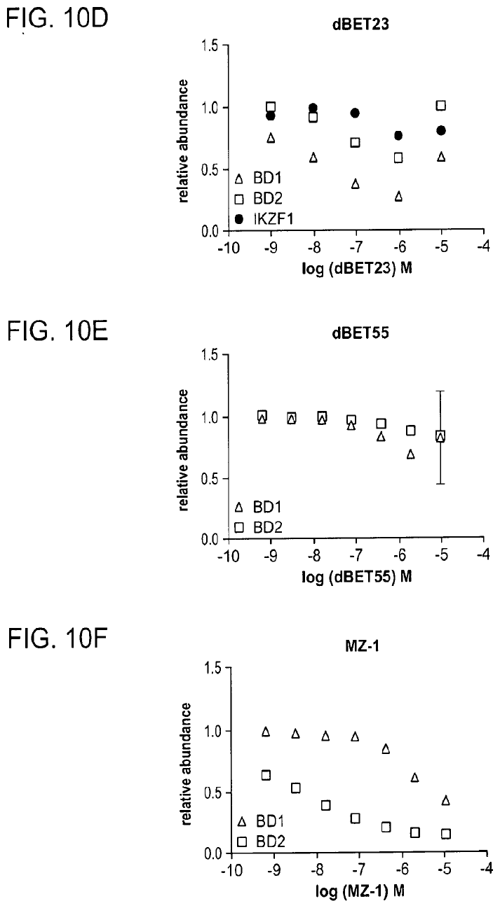

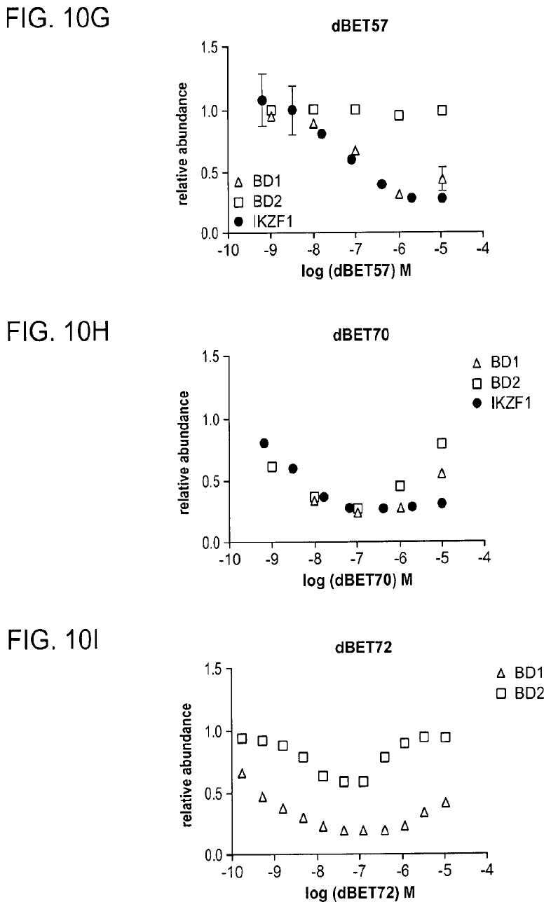

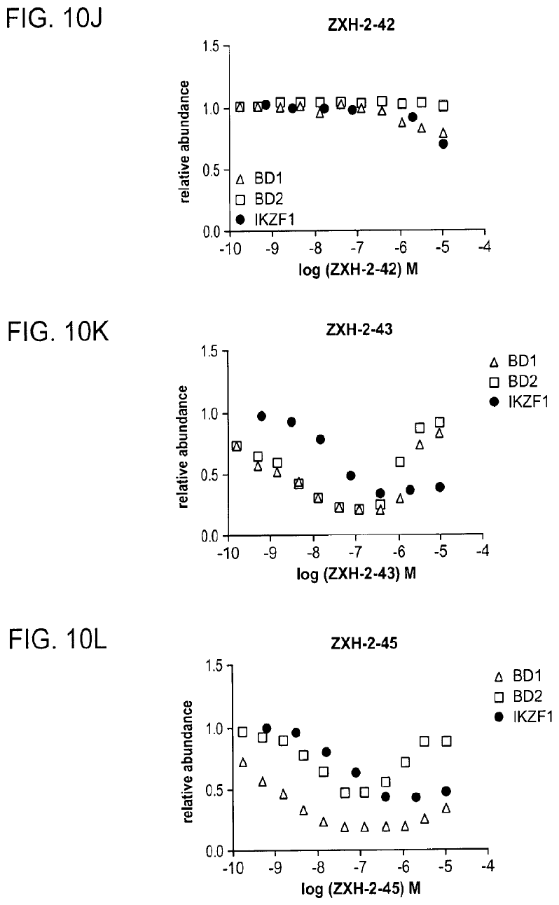

To better understand the drivers of selectivity and to test whether the observed differences in cooperativity would result in differential degradation of isolated BRD4 bromodomains, a system was developed that allowed us to directly quantify cellular degradation of either BRD4_{BD1} or BRD4_{BD2}. Reporter cells that stably express BRD4_{BD1}-EGFP followed by a P2A splice site separated mCherry, were treated with increasing concentrations of dBET molecules (FIGS. 3A-F). This assay format enables quantitative readout of BRD4_{BD1} degradation with the GFP/mCherry ratio using flow cytometry (similar reporter cells were used for BRD4_{BD2}, or a IKZF protein that has internal deletions Δ1-82, Δ197-239, and Δ256-519 hereafter referred to as IKZFΔ). The Examples demonstrate that dBET6 (DC_{50/5h}˜10 nM, with DC_{50/5h} referring to half-maximal degradation after 5 hours of treatment), dBET23 (DC_{50/5h}˜50 nM) and dBET70 (DC_{50/5h}˜5 nM) exhibit the most potent effects on BRD4_{BD1} protein levels, followed by dBET1 (DC_{50/5h}˜500 nM) and dBET57 (DC_{50/5h}˜500 nM) (FIGS. 3A-C and FIGS. 10A-L). For BRD4_{BD2}, dBET70 (DC_{50/5h}˜5 nM) has the most pronounced effects, followed by dBET6 (DC_{50/5h}˜50 nM), dBET23 (DC_{50/5h}>1 μM) and dBET (DC_{50/5h}˜1 μM), dBET57, which exhibits significant degradation of BRD4_{BD1}, is inactive on BRD4_{BD2} (FIGS. 3D-F and FIGS. 10A-L). The cellular activity is thus directly proportional to the observed cooperativity factors (FIGS. 9A-B), and dBET57 was found remarkably selective for BRD4_{BD1} in biochemical and cellular assays (FIG. 2F and FIGS. 3A-F).

### Example 4: Plastic Binding Confers Selectivity to dBETs

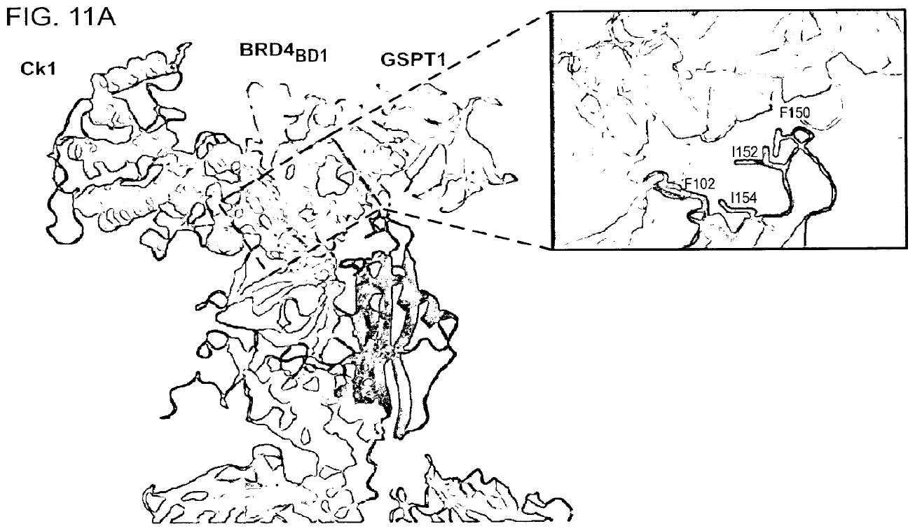

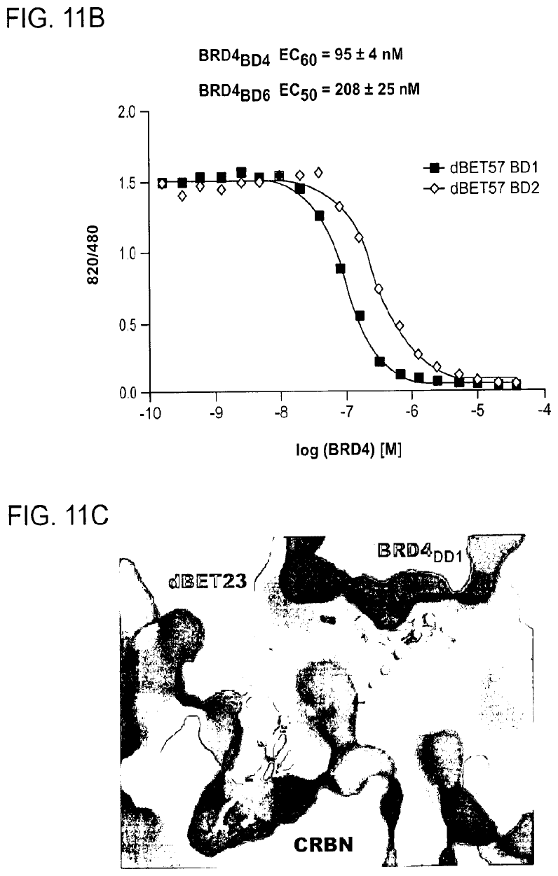

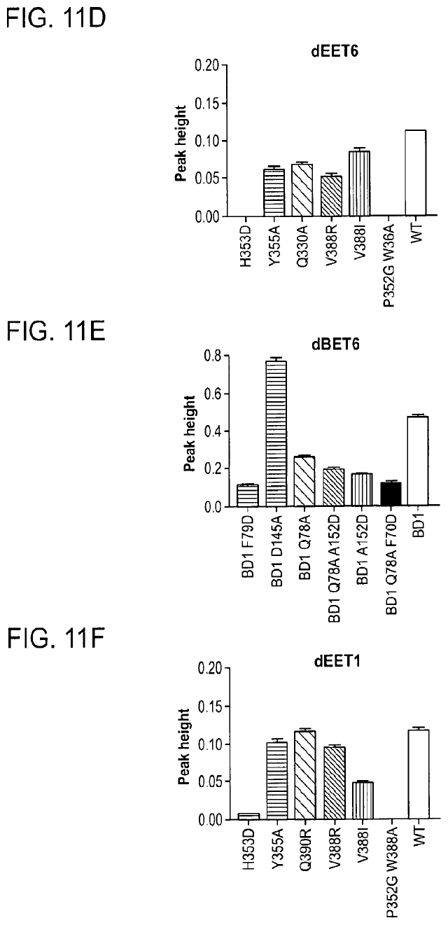

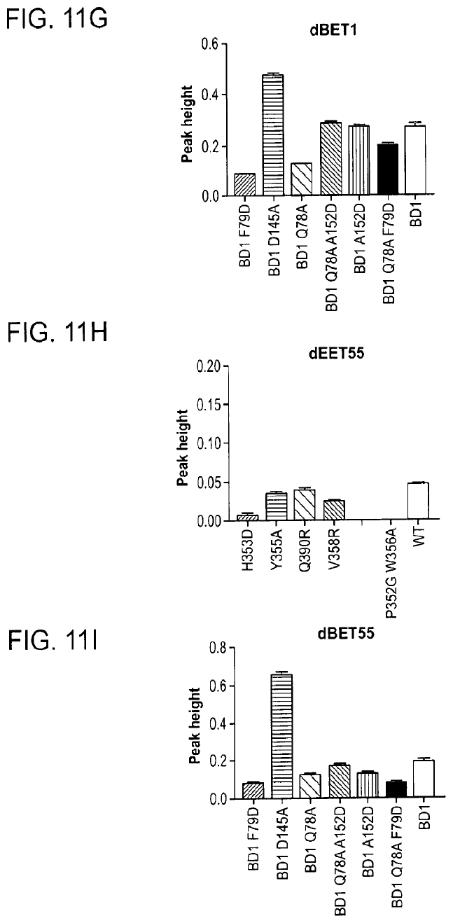

When comparing the CRBN-dBET23-BRD4_{BD1} structure to the previously determined structures of CRBN-Ck1α (Petzold, Fischer et al. 2016), and CRBN-GSPT1 (Matyskiela, Lu et al. 2016), the Examples show that these neo-substrates use different surfaces on CRBN to stabilize tertiary complex formation (FIG. 11A). The Examples also show that molecules with short linkers, such as dBET57, would not be able to dimerize CRBN and BRD4 in the conformation observed in the CRBN-dBET23-BRD4_{BD1} structure since a minimum of 8 carbons would be required to bridge the E3-moeity with the target-moiety and dBET57 comprises a 2-carbon linker (FIG. 11C). Additional Examples address whether dBET molecules incompatible with the observed binding mode, such as dBET57 or dBET1, would bind in a different overall conformation.

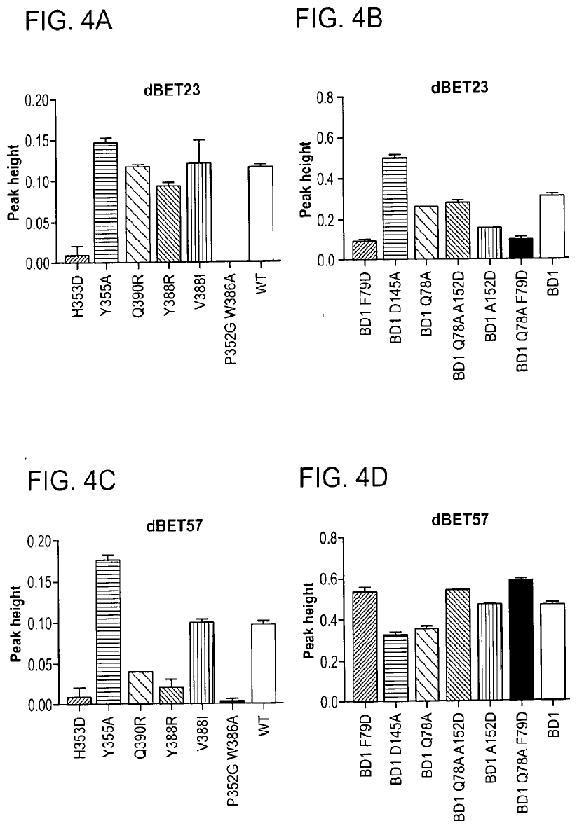

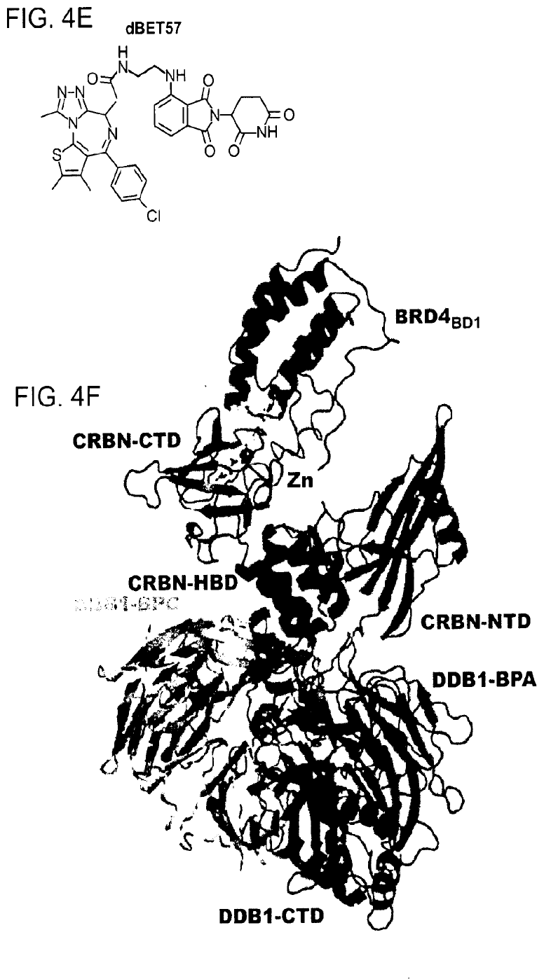

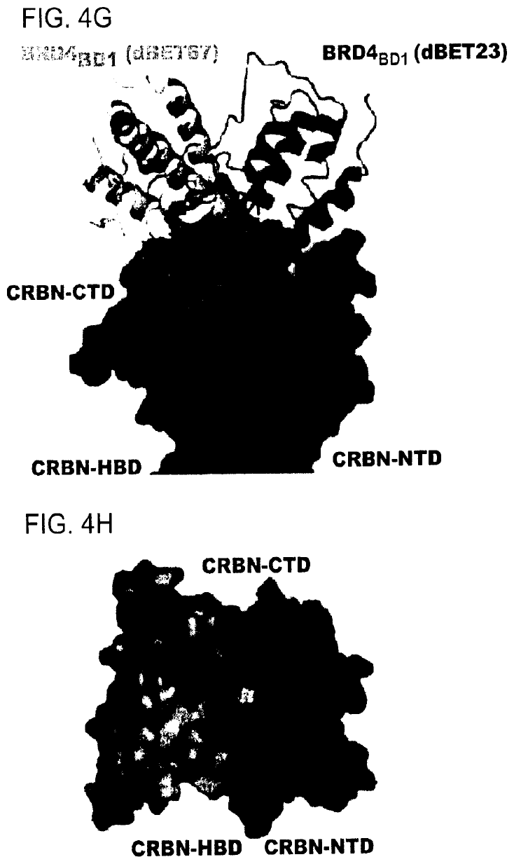

To explore potential differences in binding, mutational analysis was performed. A set of single amino acid point mutations was introduced in CRBN and BRD4_{BD1} to obtain a mutational signature of binding. When comparing the mutational signatures of different dBETs, the Examples show that while dBET6 and 23 share similar profiles (FIGS. 4A and B, and 11D and E), the mutational signatures of dBET and dBET57 are distinct (FIGS. 4A-D and 11D-I). This suggests that different dBET molecules—depending on linker length and linkage position—result in distinct binding conformations of CRBN-BRD4 complex formation.

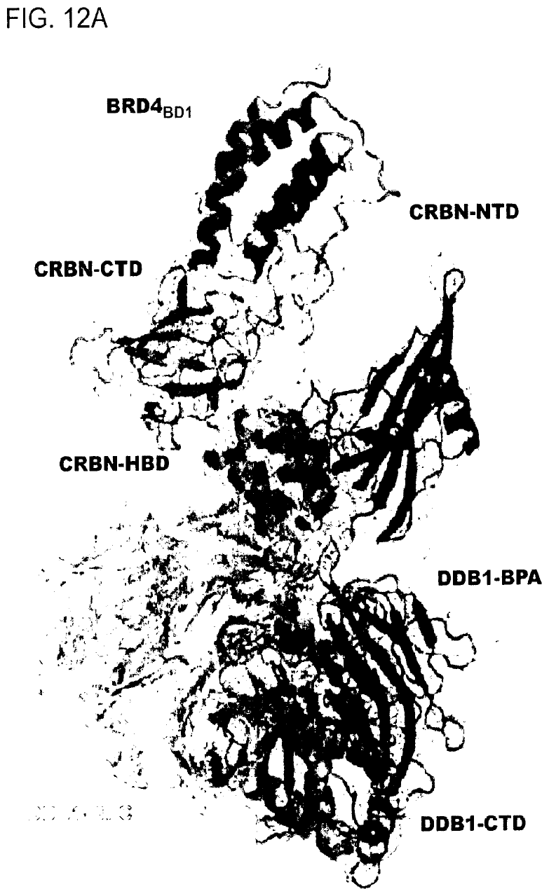

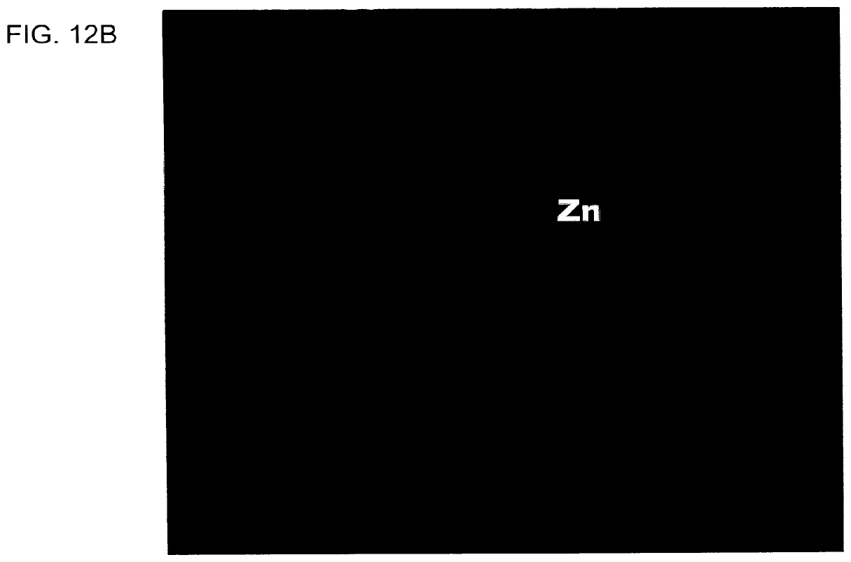

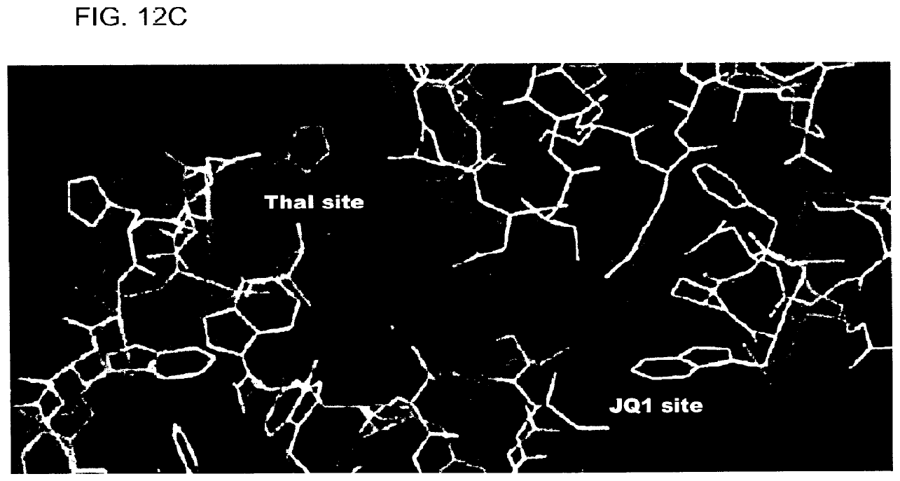

To obtain insights into the molecular basis of this plastic CRBN/BRD4_{BD1} interactions, dBET57 (the molecule with the most pronounced selectivity for BRD4_{BD1} over BRD4_{BD2}.) was crystallized. Crystals were obtained for a reconstituted DDB1ΔB-CRBN-dBET57-BRD4_{BD1} complex and determined the structure to 6.8 Å resolution (see FIGS. 12A-C for experimental validation of the structure). While the limited resolution prevents detailed interpretation of the molecular interactions that govern the CRBN-BRD4 interface, the overall binding mode is clearly resolved (FIGS. 4F and 12A). In this complex, BRD4_{BD1} interacts with the CTD of CRBN, instead of the NTD as observed with dBET6/23 (FIGS. 4E-H), which results in BRD4 now utilizing an entirely different set of residues for inter-protein contexts (compare FIG. 2B and FIG. 4H). In the dBET57 bound structure, the Examples show that CRBN unfolds and the CRBN-NTD and CRBN-CTD domains no longer interact (FIGS. 4E-F). This unexpected behaviour could be due to the high salt crystallization condition (1.6 M Phosphate) or part of the intrinsic CRBN plasticity. The binding mode observed with dBET57, however, is fully compatible with a regular CRBN conformation (FIG. 4G) and dBET57 mediated binding thus expected to occur with both CRBN conformations (see FIGS. 12A-C). The unexpected plasticity in dBET dependent binding of CRBN to the exact same protein, BRD4_{BD1}, provides a rationale how PROTACs that share the same E3- and target-moieties can still exhibit different selectivity profiles. Depending on the linker, different surface residues in the target protein may be involved in complex formation.

FIG. 12A shows that CRBN was found in a not previously observed conformation, in which the thalidomide binding CRBN-CTD domain translates and rotates away from the CRBN-HBD and CRBN-NTD domains. This results in an open conformation that exposes large areas of CRBN that are typically buried. The high salt crystallization condition could be a driver of this structural rearrangement, and together with crystal contacts induce this conformation. It is possible that that this conformational dynamic is an intrinsic feature of CRBN to accommodate a variety of substrates and future studies are necessary to address this. Based on the compatibility of the observed BRD4_{BD1} binding conformation with the open and closed CRBN conformations, it can be concluded that for the interpretation of the data, the conformational change is negligible.

### Example 5: Protein Docking Reveals Binding Energy Landscape

The mutational signatures obtained for different dBET molecules, the structural arrangements for dBET6/23/70 and dBET57 complexes, together with the absence of any co-evolution between CRBN and BRD4 let us hypothesize that BRD4 bromodomains can bind to CRBN in multiple different orientations depending on the ligand. Assessing such potential binding conformations to reduce chemical search space would be highly desirable. In silico protein-protein docking provides an attractive surrogate to in vitro experiments. The Examples addressed whether the Rosetta protein docking framework (Sircar, Chaudhury et al. 2010) would allow modelling of such possible binding modes. One of the characteristics of Monte-Carlo docking algorithms is the stochastic sampling of low energy conformations, which frequently results in multiple solutions. While this often complicates the identification of evolved interactions between proteins, sampling of possible conformations provides an advantage in the study of degrader-induced binding modes since it enables exploration of the repertoire of low energy conformations.

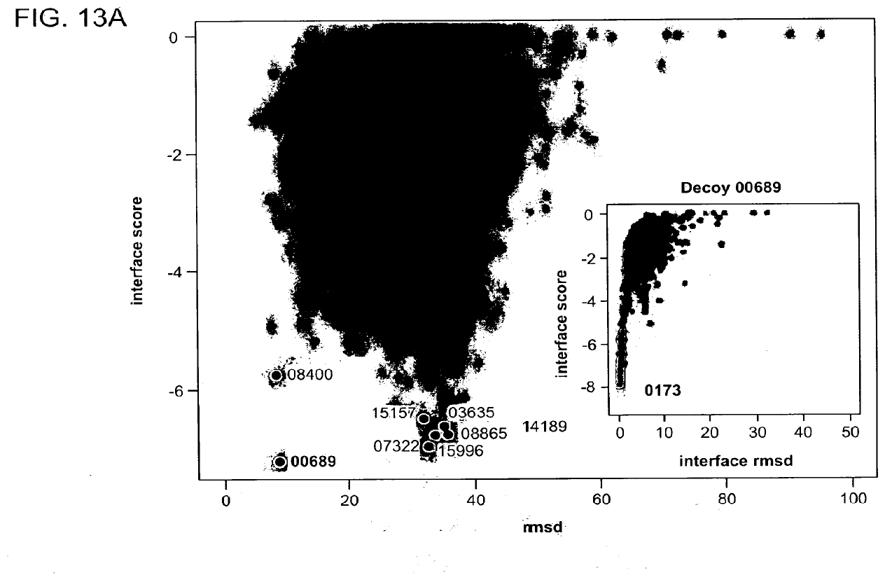

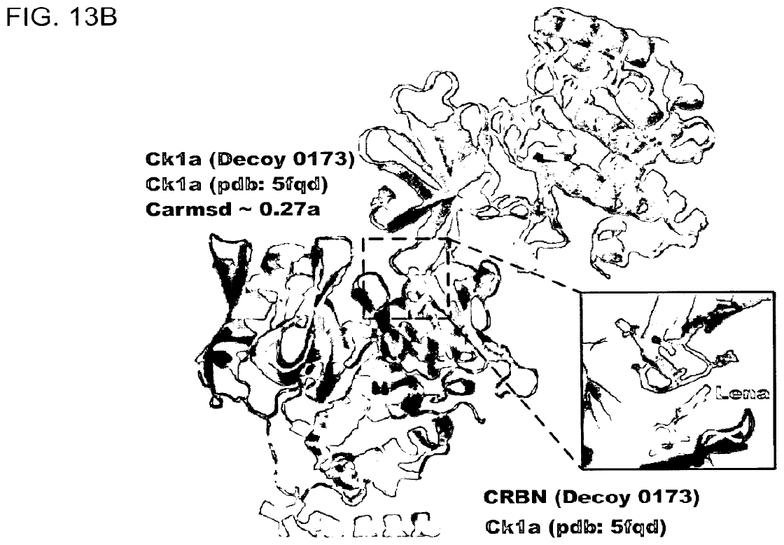

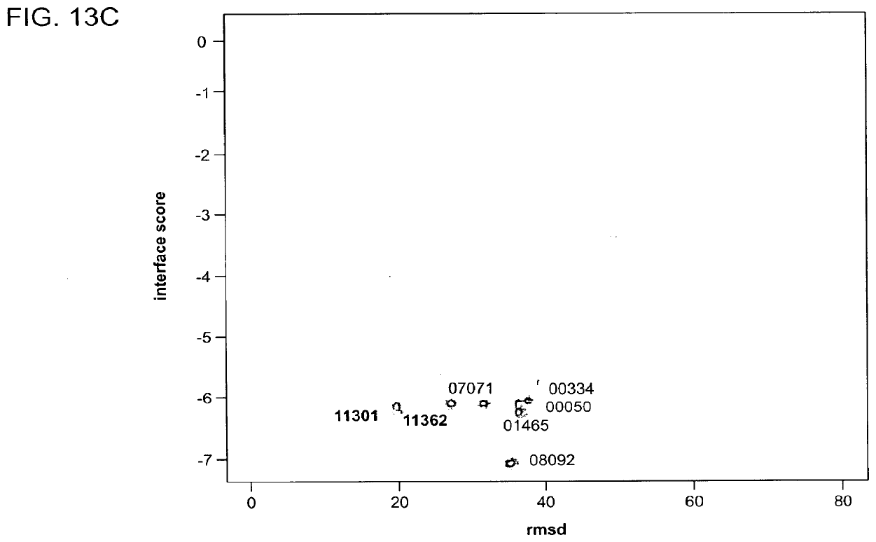

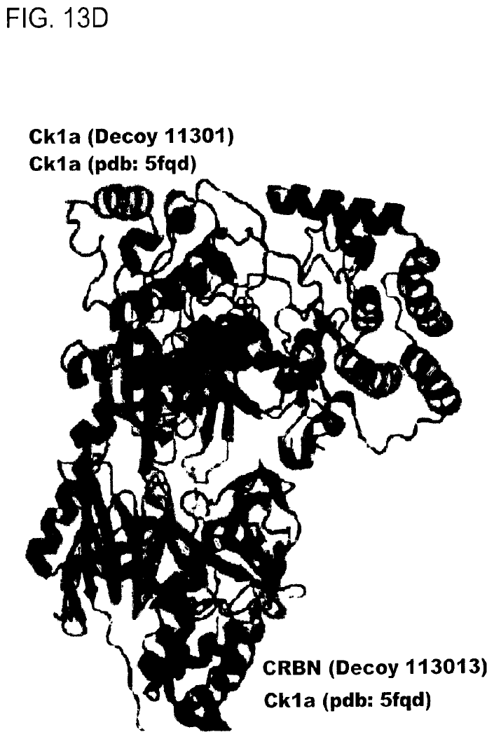

The Examples confirmed that computational methods can predict ligand mediated protein-protein interactions by docking Ck1α to the CRBN-lenalidomide complex (FIGS. 13A-D; “molecular glue docking”). The Examples further addressed whether computational docking would be able to provide models for possible PROTAC-induced binding modes by docking CRBN and the target BRD4_{BD1} in absence of dBET. One obvious complication is that a dominant component of the binding energy between ligase and substrate is provided by the degrader itself, which is absent in docking simulations, and the scoring of solely neomorphic interactions will likely result in many low energy conformations to be generated.

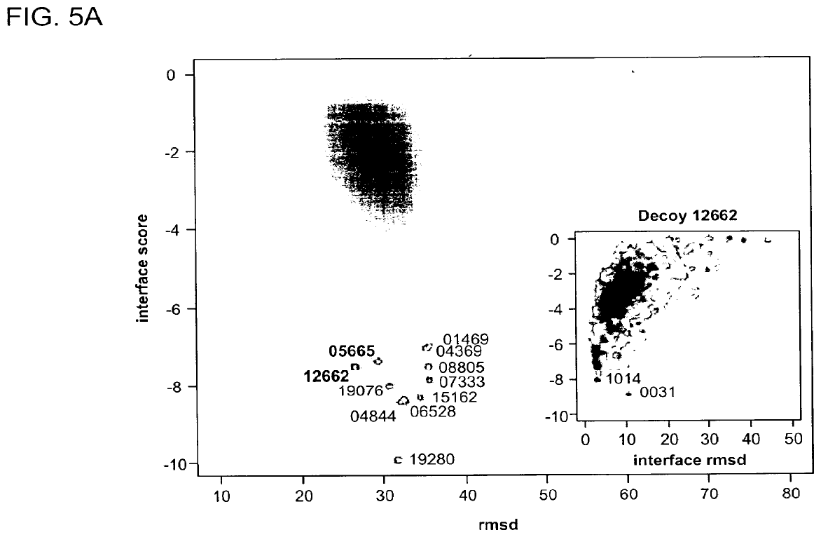

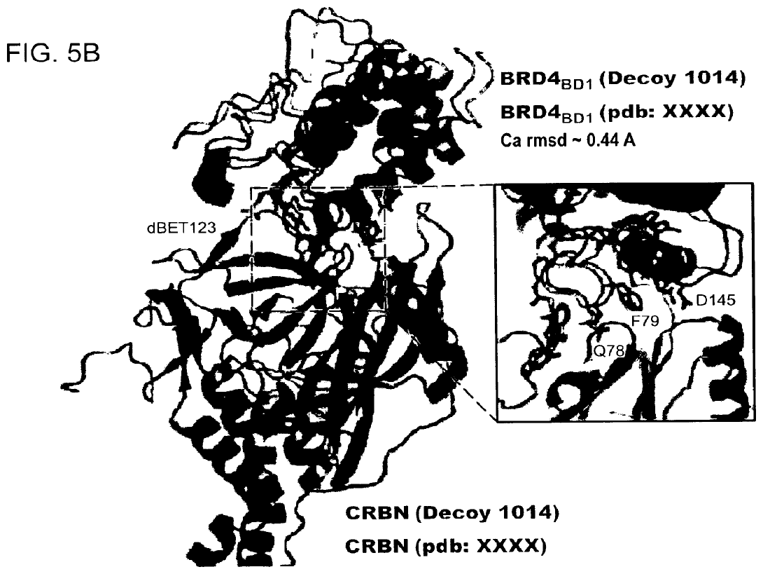

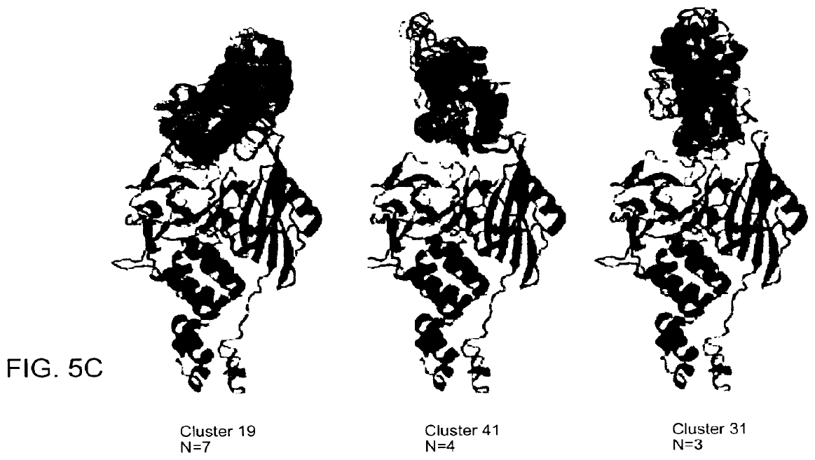

Using the crystal structure of lenalidomide bound CRBN (pdb: 4tz4) and JQ1 bound BRD4_{BD1} (pdb: 3mxf), a global docking experiment (20,000 models) was performed using Rosetta docking (FIG. 5A). Clustering the top 200 lowest scoring docking conformations, a conformation was identified that closely resembles the conformation observed in the dBET23 crystals. This model was further confirmed by local docking (2,000 models) of the low energy model (FIGS. 5A and B).

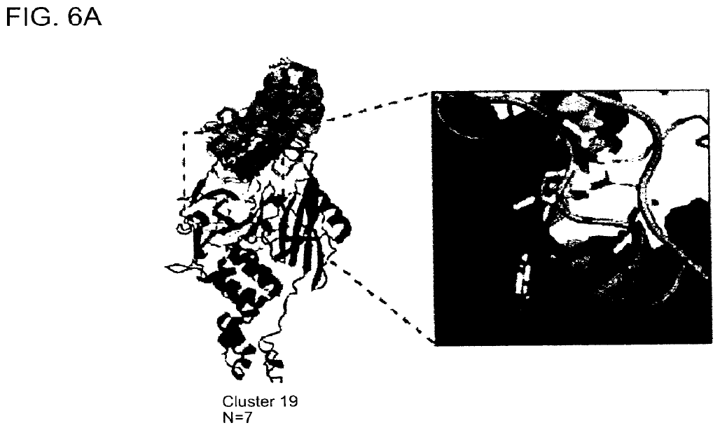

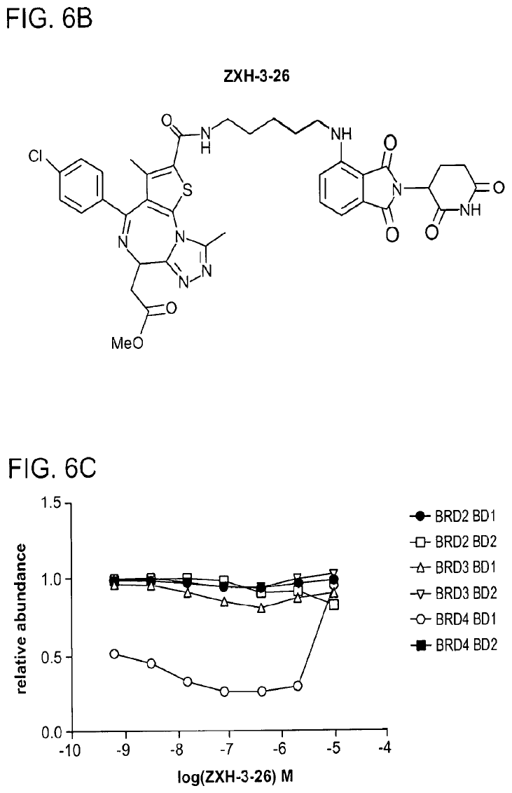

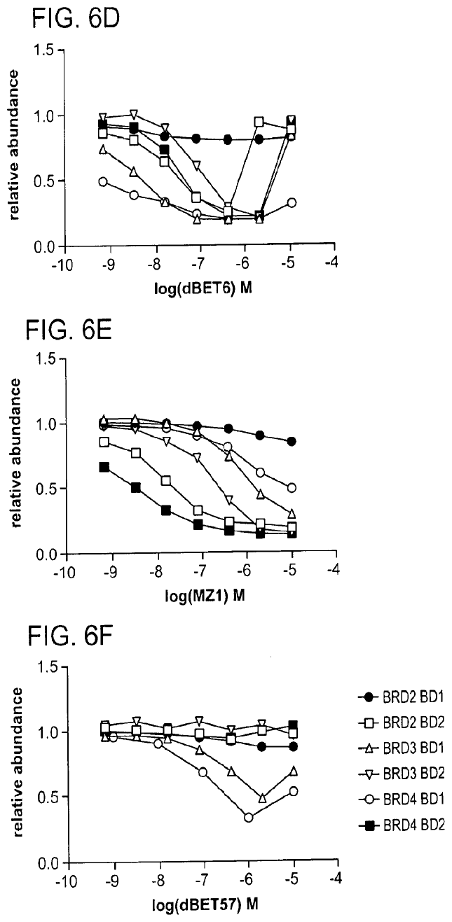

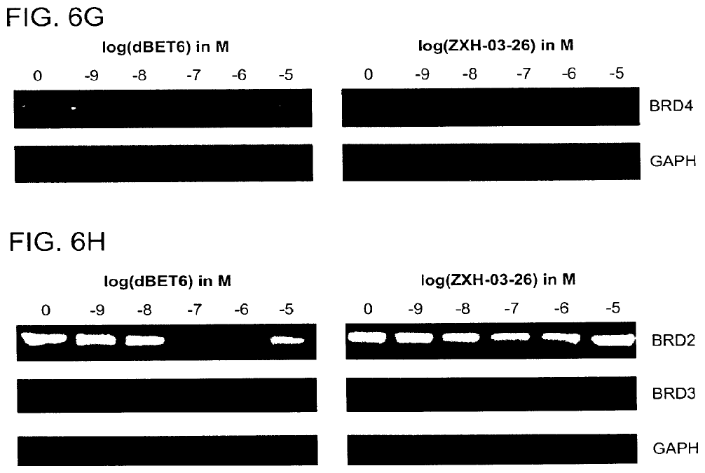

As predicted for a much weaker interaction between CRBN and BRD4_{BD1} in absence of a degrader, multiple low energy minima are found. Based on the hypothesis that the docking experiment will sample the repertoire of low energy binding conformations, clustering of the top 200 conformations provides a set of feasible binding modes (see FIG. 5C) for representative clusters). While it remains to be shown whether docking can predict binding modes accurately, the overall conformational landscape provides a rationale for the design of required minimal linker lengths and suggest suitable linkage positions. In theory, the shortest possible linker for a ligase-target pair should provide the most selective compound since it will restrict the number of possible binding conformations. To test whether the docking information could be used to inform the design of PROTACs, poses were sorted by minimal required linker length between the JQ1 thiophene and lenalidomide, and found a linker of 2-3 atoms sufficient to bridge the two moieties (FIG. 6A). The according molecules (ZXH-02-147 and ZXH-03-26) were synthesized (FIGS. 6B and 7B).

The Examples addressed whether certain degraders (PROTACs) would be capable of directly inducing binding of IKZF1 (and other IMiD targets) to CRBN. A CRBN-IKZF1Δ binding assay was used to measure binding of IKZF1Δ to CRBN in presence of dBET1, dBET6, dBET23, dBET57, dBET70, and dBET72, as well as lenalidomide as control (FIG. 14A). The Examples show that dBET1/6/23 do not induce IKZF1-CRBN complex formation, while dBET57, dBET70 and dBET72 show pronounced complex formation. Both, dBET57 and dBET70 share the aniline of lenalidomide, while dBET1/6/23 all have an oxy-acetamide linkage. Based on the previously described model of IKZF1-CRBN binding (FIG. 14C) the phthalimide aniline nitrogen may be involved in a hydrogen bond with IKZF1 Q146. A straight linker out of this phthalimide position could be tolerated, while an adjacent amide bond (as in the oxy-acetamide linkage) may cause a steric clash with IKZF1. Alternatively, the secondary amine nitrogen could be a hydrogen bond donor and, with the ether oxygen being a hydrogen bond acceptor, this donor/acceptor substitution could explain the difference in strength of the IKZF1 interaction. The nitrogen linkage of dBET57, dBET70 and dBET72 were replaced with an oxygen-ether linkage resulting in compounds ZXH-2-42, ZXH-2-43, and ZXH-2-45, respectively. The ability of the oxygen-ether compounds to induce binding of IKZF1 was greatly reduced compared to their nitrogen analogs; however, it was not eliminated, as seen in the case of the oxy-acetamide substitution.

### Example 6: Degradation of an IKZF1Δ-EGFP Fusion Protein

Dose dependent degradation of an IKZF1 A-EGFP fusion protein was assessed in HEK293T cells (see methods), and used the in vitro structure activity relationship (SAR) to develop a model of cellular IKZF1 degradation (FIG. 14B), dBET1/6/23 are relatively ineffective at promoting IKZF1 degradation, dBET70/72 are equipotent to lenalidomide, and dBET57 is comparable to thalidomide, in accordance with the biochemical data. The Examples show that by modifying the substitution at the IMiD moiety, the co-degradation of other substrates—such as IKZF1—can be controlled or modulated. To test whether this would be effective in a cellular multiple myeloma model, MM.1s cells were treated for five hours with either 1 μM dBET23, 1 μM dBET70 or DMSO as a control. Using a quantitative proteomics approach (see methods), the Examples demonstrate that dBET70 but not dBET23 exhibits pronounced co-degradation of CRBN-lenalidomide neo-substrates IKZF1, IKZF3 and ZFP91 (FIGS. 14D and E).

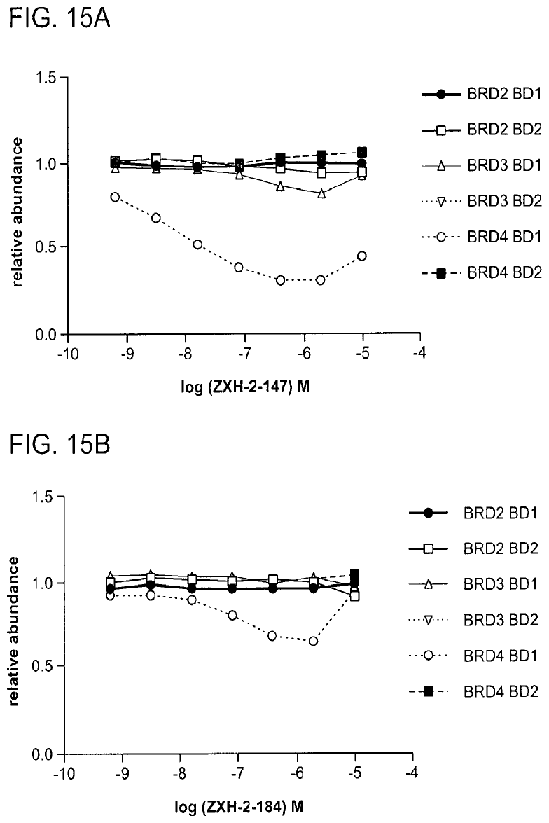

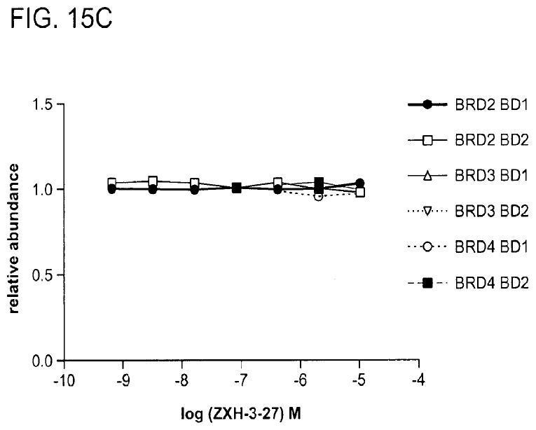

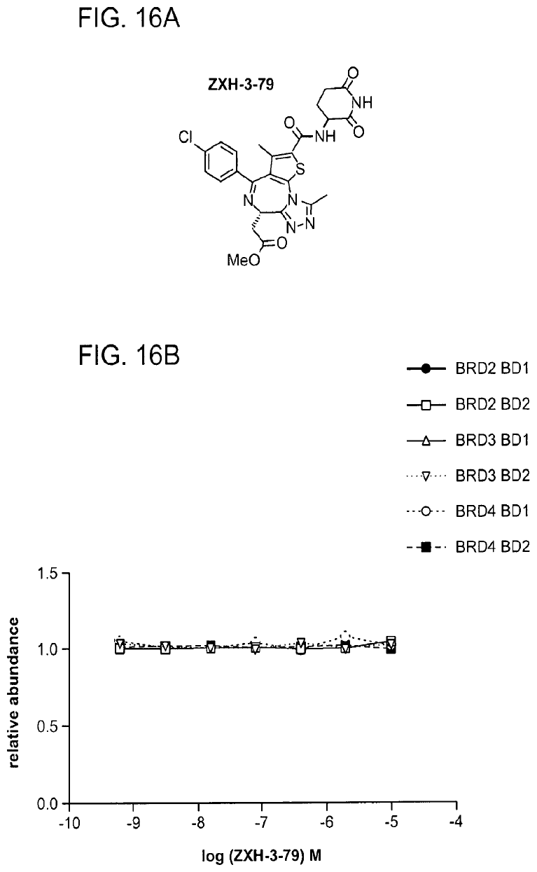

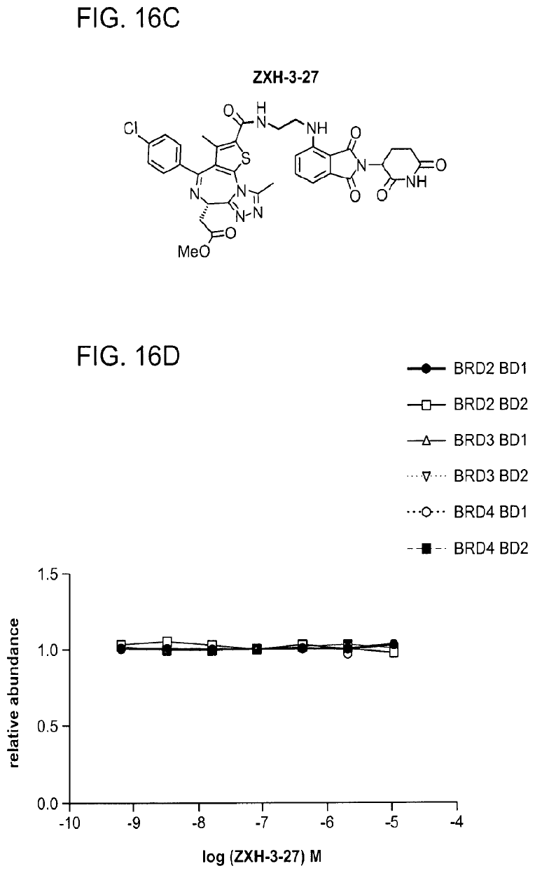

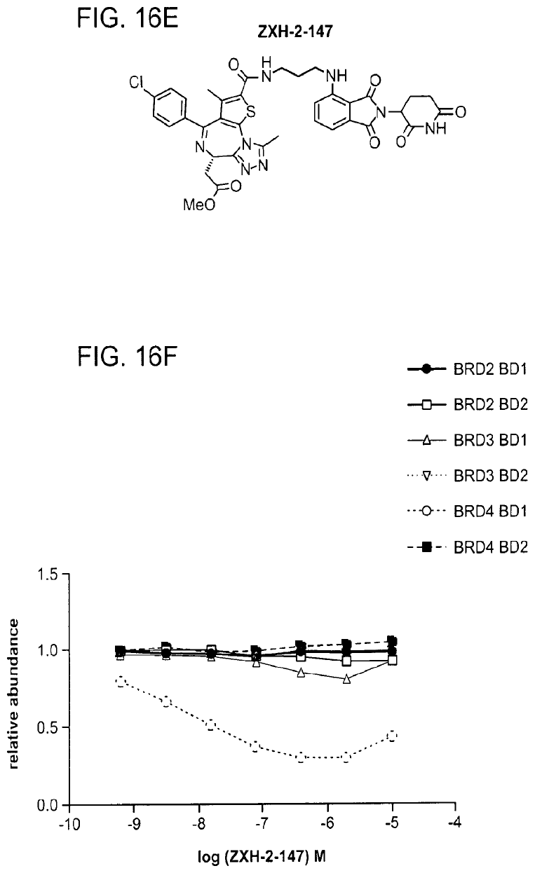

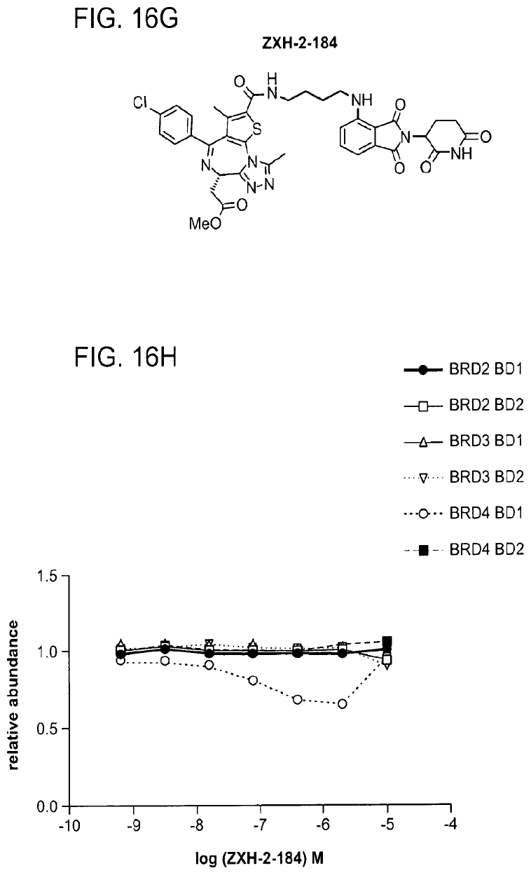

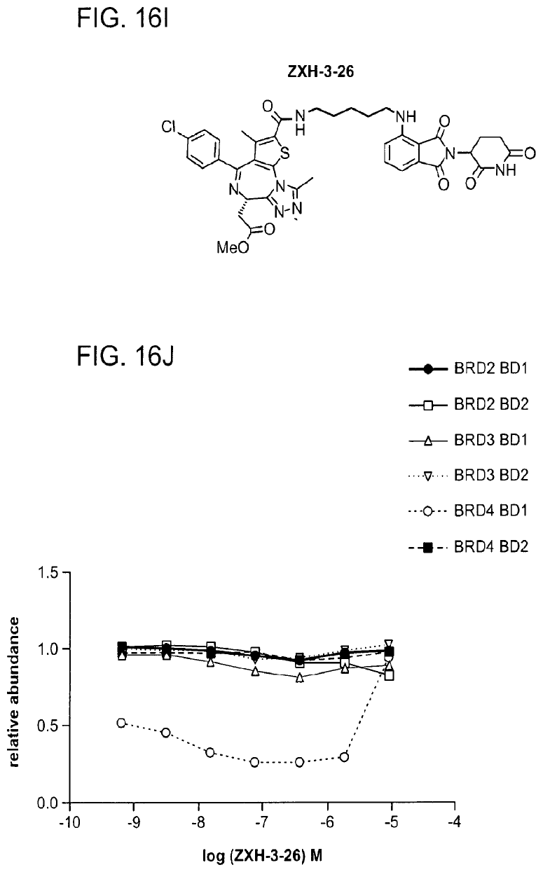

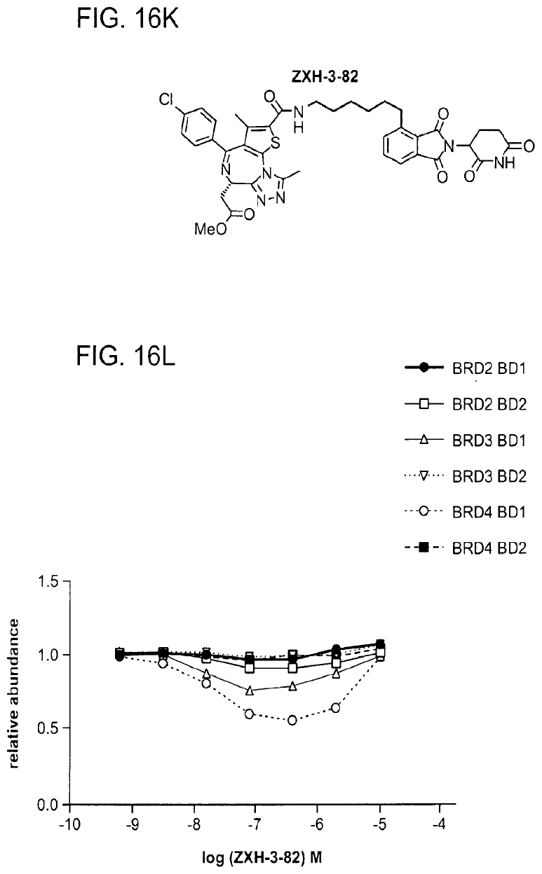

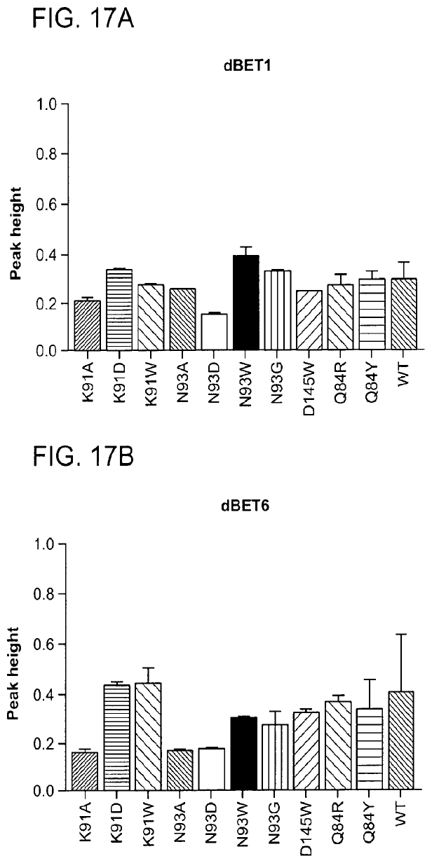

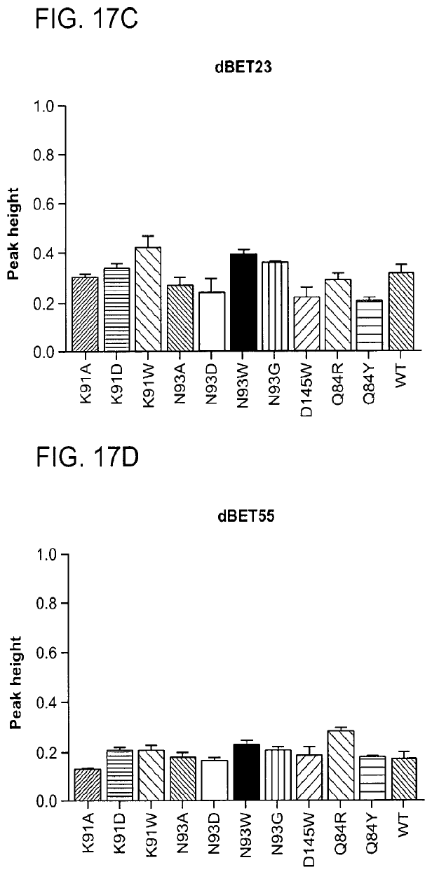

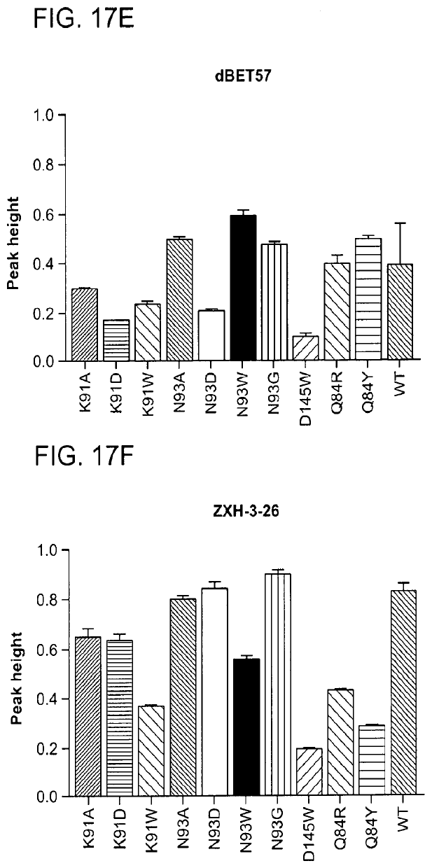

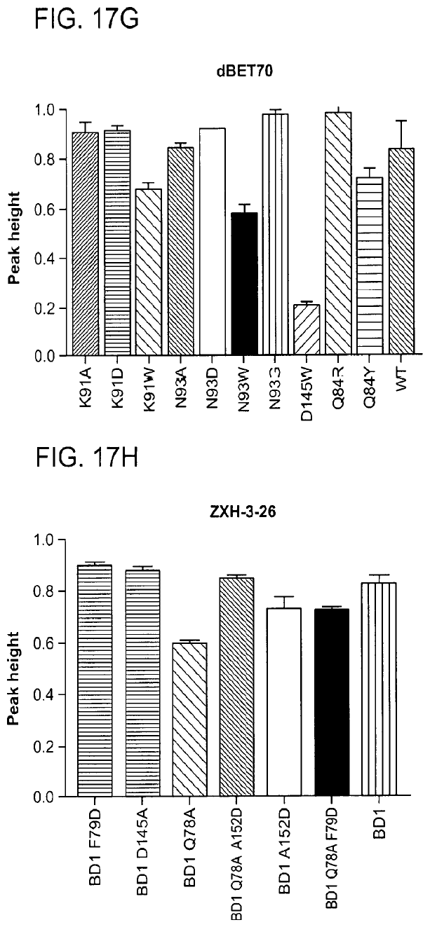

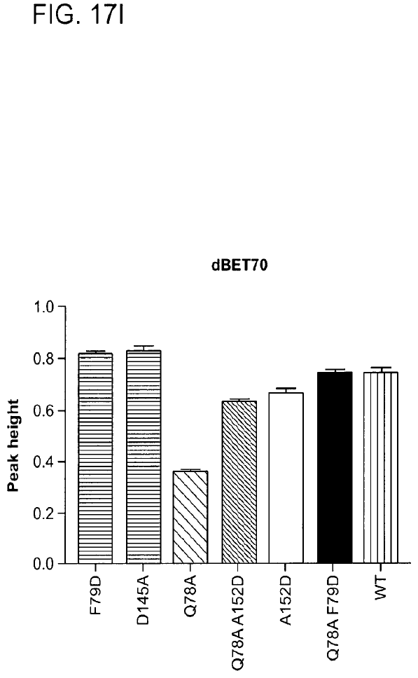

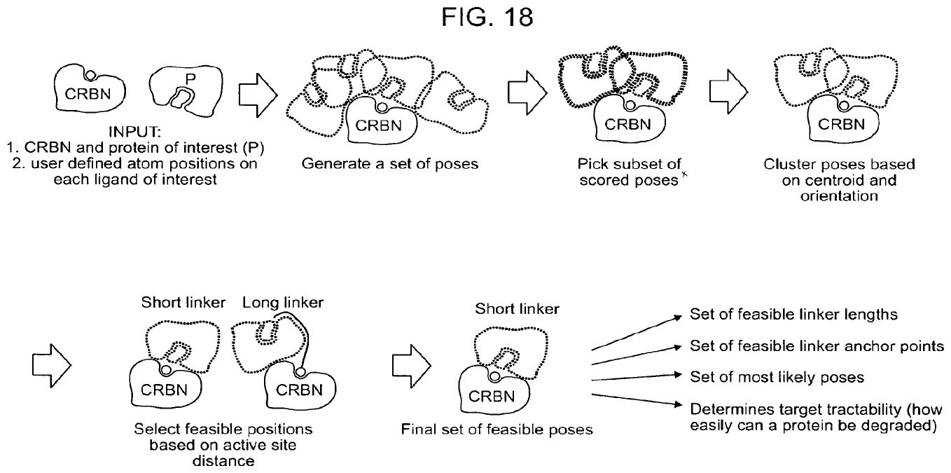

Cellular degradation assays show that ZXH-02-147 and ZXH-03-26 are active on BRD4_{BD1}, in accordance with the docking results (FIGS. 6C and 15A), and that ZXH-03-26 exhibits a DC_{50/5h}˜5 nM comparable to the best pan-BRD degrader dBET6. To test whether these molecules exhibit isoform selectivity, the cellular reporter system was expanded to include the individual bromodomains of BRD2 and BRD3 and tested cellular degradation along with BRD4. The Examples show that ZXH-03-26 shows activity exclusively on the first bromodomain of BRD4, and spares degradation of BRD2 or 3 at concentrations >10 μM (FIG. 6C), while dBET6 and MZ1 as controls show activity on most bromodomains (FIG. 6D). Next, bromodomain degradation for dBET57 was assessed to test whether any short linker would result in selectivity for BRD4_{BD1}. In contrast to ZXH-03-26, dBET57 is nearly equipotent on BRD3_{BD1} and BRD4_{BD1} (FIG. 6E). To test whether the selective ZXH-03-26 retains activity on endogenous full length BRD4, HEK293T cells were treated with increasing concentrations of ZXH-03-26. Immunoblot analysis confirms that ZXH-03-26 degrades endogenous BRD4 with comparable efficacy compared to the best pan-BET degrader dBET6 (FIG. 6G), while being inactive on BRD2 and BRD3 (FIG. 6H). ZXH-03-26 thus demonstrates that binding to a distinct conformation can yield a highly selective degrader molecule and that selectivity can be achieved across highly homologous domains such as the bromodomains of BET proteins.

### Example 7: Constructs and Protein Purification

Wild-type and mutant versions of human DDB1, human CRBN, and human IKZF1Δ were cloned in pAC-derived vectors (Abdulrahman, Uhring et al. 2009) and recombinant proteins were expressed as N-terminal His₆ (DDB1ΔB, CRBN), StrepII-Avi (IKZF1Δ) or hiss-3C-Spy (CRBN) (Zakeri, Fierer et al. 2012) fusions in Trichoplusia ni High-Five insect cells using the baculovirus expression system (Invitrogen). Wild-type and mutant BRD4_{BD1} and BRD4_{BD2} subcloned into E. coli pET100/D-TOPO vector with N-terminal His₆-Avi fusions were obtained from Invitrogen, BRD4_{BD1/2} were subcloned into N-terminal his₆-MBP-TEV-Spy pETDuet vector and all expressed in BL21-DE3 or BL21-DE3 Rosetta cells using standard protocols. For purification of His₆ and GST tagged proteins, cells were resuspended in buffer containing 50 mM tris (hydroxymethyl)aminomethane hydrochloride (Tris-HCl) pH 8.0, 200 mM NaCl, 1 mM tris (2-carboxyethyl)phosphine (TCEP), 1 mM phenylmethylsulfonyl fluoride (PMSF), 1× protease inhibitor cocktail (Sigma) and lysed by sonication. Cells expressing StrepII-Avi-IKZF1Δ were lysed in the presence of 50 mM Tris-HCl pH 8.0, 500 mM NaCl, 1 mM TCEP, 1 mM PMSF and 1× protease inhibitor cocktail (Sigma). Following ultracentrifugation, the soluble fraction was passed over appropriate affinity resin Strep-Tactin Sepharose (IBA) or Ni Sepharose 6 Fast Flow affinity resin (GE Healthcare) or Glutathione Sepharose 4B (GE Healthcare) and eluted with wash buffer (50 mM Tris-HCl pH 8.0, 200 mM NaCl, 1 mM TCEP) supplemented with 2.5 mM D-Desthiobiotin (IBA) or 100 mM imidazole (Fischer Chemical) or 10 mM glutathione (Fischer BioReagents) respectively. The affinity-purified protein was either further purified (CRBN-DDB1ΔB, IKZF1Δ, Spy-BRD4_{BD1}) via ion exchange chromatography (Poros 50HQ) and subjected to size exclusion chromatography or concentrated and directly loaded on the size exclusion chromatography in 50 mM HEPES pH 7.4, 200 mM NaCl and 1 mM TCEP. Biotynylation of IKZF1Δ and BRD4_{BD1}, BRD4_{BD2} variants was performed as previously described (Petzold, Fischer et al. 2016).

The protein-containing fractions were concentrated using ultrafiltration (Millipore) and flash frozen in liquid nitrogen (DDB1ΔB-CRBN constructs at 40-120 μM, biotinylated His₆-Avi-BRD4 mutants and WT, and not biotinylated WT at ˜25-100 μM, biotinylated StrepII-Avi-IKZF1 at ˜20 μM concentration) and stored at −80° C. or directly covalently labelled with BODIPY-FL-SpyCatcherssoc (His₆-3C-Spy-CRBN-His₆-DDB1ΔB, Spy-BRD4_{BD1}) as described below.

### Example 8: SpyCatcher S50C Mutant

Spycatcher containing a Ser50Cys mutation was obtained as synthetic dsDNA fragment from IDT (Integrated DNA technologies) and subcloned as GST-TEV fusion protein in a pET-Duet derived vector. Spycatcher S50C was expressed in BL21 DE3 and cells were lysed in the presence of 50 mM Tris-HCl pH 8.0, 200 mM NaCl, 1 mM TCEP and 1 mM PMSF. Following ultracentrifugation, the soluble fraction was passed over Glutathione Sepharose 4B (GE Healthcare) and eluted with wash buffer (50 mM Tris-HCl pH 8.0, 200 mM NaCl, 1 mM TCEP) supplemented with 10 mM glutathione (Fischer BioReagents). The affinity-purified protein was subjected to size exclusion chromatography, concentrated and flash frozen in liquid nitrogen.

### Example 9: Labelling of Spycatcher with BODIPY-FL-maleimide

Purified Spycatcher_{S50c} protein was incubated with DTT (8 mM) at 4° C. for 1 h. DTT was removed using a ENRich SEC650 10/300 (Bio-rad) size exclusion column in a buffer containing 50 mM Tris pH 7.5 and 150 mM NaCl, 0.1 mM TCEP. BODIPY-FL-maleimide (Thermo Fisher) was dissolved in 100% DMSO and mixed with Spycatcher_{S50c} to achieve 2.5 molar excess of BODIPY-FL-maleimide. SpyCatcherssoc labelling was carried out at room temperature (RT) for 3 h and stored overnight at 4° C. Labelled Spycatcher_{S50c} was purified on a ENRich SEC650 10/300 (Bio-rad) size exclusion column in 50 mM Tris pH 7.5, 150 mM NaCl, 0.25 mM TCEP and 10% (v/v) glycerol, concentrated by ultrafiltration (Millipore), flash frozen (˜40 μM) in liquid nitrogen and stored at −80° C.

### Example 10: BODIPY-FL-Spycatcher Labelling of CRBN-DDB1ΔB and BRD4_{BD1}

Purified His₆-DDB1ΔB-His₆-3C-Spy-CRBN or His₆-Spy-BRD4_{BD1} was incubated overnight at 4° C. with BODIPY-FL labelled SpyCatcherssoc protein at stoichiometric ratio. Protein was concentrated and loaded on the ENrich SEC 650 10/300 (Bio-rad) size exclusion column and the fluorescence monitored with absorption at 280 and 490 nm. Protein peak corresponding to the labeled protein was pooled, concentrated by ultrafiltration (Millipore), flash frozen (˜9.6 μM for His₆-DDB1ΔB-His₆-3C-Spy-CRBN_{BODIPY SpyCatcher} or ˜22 uM for His₆-Spy-BRD4_{BD1}) in liquid nitrogen and stored at −80° C.

### Example 11: Crystallization and Data Collection

Previously developed DDB1 construct was used that lack WD40 propeller B (BPB, residues 396-705) domain (Petzold, Fischer et al. 2016) (referred to as DDB1ΔB) successful in crystallization of lenalidomide-CK1α complex. For crystallization of His₆-DDB1ΔB-His₆-CRBN-dBET6/23/70-his₆-BRD4_{BD1} and His₆-DDB1ΔB-His-CRBN-dBET55-His_{6-Avi}-BRD4_{BD1 D145A} complexes 145 μM of dBET was mixed with 70 μM BRD4_{BD1} or BRD4_{BD1 D145A} and 80 μM His₆-DDB1ΔB-His₆-CRBN and incubated for 15 min either on ice or at RT. Crystallisation plates were set up in 3 sub-well plates (Intelli, Art Robbins) by vapour diffusion using NT8 (Formulatrix) at 20° C. and images acquired using RockImager 1000 (Formulatrix). Crystals appeared in wells B9-F9 and H9 of Morpheus HT Screen (Molecular Dimensions) within few hours and were fully grown after 3 days. Single uniform crystals (length 80-100 μm) were present in condition C9 (10% (w/v) PEG20k, 20% (w/v) PEG550 MME, 0.1 M BICINE pH 8.5) in 2:1 or 1:1 protein to precipitant ratio in 150 or 225 nL drops. Further optimisation of condition in Morpheus HT Screen C9 by SilverBullet (Hampton Research) additive screening in 1:10 additive to reservoir ratio resulted in optimal crystals for dBET6, dBET23, dBET55 and dBET70 in Silver Bullet wells D7, B5, G4 and F6 respectively, in 2:1 protein to precipitant ratio of 225 or 400 nL drops. Crystals were cryo-protected in reservoir solution supplemented with 25-30% PEG 400 containing 150-300 μM respective dBET and flash-cooled in liquid nitrogen. The Examples show that crystals harvested after 2-3 days resulted in optimal diffraction. Diffraction data were collected at the APS Chicago (beamline 24-ID-C) with a Pilatus 6M-F detector at a temperature of 100 K, or for dBET6 co-crystal structure at beamline 24-ID-E with a Eiger 16M detector at a temperature of 100 K. Data were indexed and integrated using XDS (Kabsch 2010) and scaled using AIMLESS supported by other programs of the CCP4 suite (Winn, Ballard et al. 2011) or RAPD pipeline (APS Chicago). Data processing statistics, refinement statistics and model quality parameters are provided in Table 1.

dBET57 containing crystals were obtained by mixing His₆-DDB1ΔB-His₆-CRBN at 75 μM, with dBET57 at 140 μM and BRD4_{BD1} at 140 μM in condition B5 of the Hampton Index HT screen (1.26 M NaH₂PO₄, 0.14 M K₂HPO₄). Single crystals were harvested, stabilized by addition of 25% ethylene glycol containing dBET57 at 50 μM. Diffraction data were collected at the APS Chicago (beamline 24-ID-C) with a Pilatus 6M-F detector at a temperature of 100 OK, at wavelengths of 0.9962 Å for native, 1.2828 Å for Zn peak, and 1.7712 for S peak. Data were indexed and integrated using XDS (Kabsch 2010) and scaled using AIMLESS supported by other programs of the CCP4 suite (Winn, Ballard et al. 2011). Data processing statistics, refinement statistics and model quality parameters are provided in Table 2.

### Example 12: Structure Determination and Model Building

The DDB1ΔB-CRBN-dBET6/23/70-BRD4_{BD1} and DDB1ΔB-CRBN-dBET55-BRD4_{BD1/D145A} quaternary complexes crystallized in space group P6₅22 with single complex in the unit cell. PHASER (McCoy, Grosse-Kunstleve et al. 2007) was used to determine the structures by molecular replacement using a crystallographic model of DDB1ΔB-CRBN omitting Ck1α based on a crystal structure PDB 5fqd. The initial model was iteratively improved with COOT and refined using PHENIX.REFINE (Afonine, Grosse-Kunstleve et al. 2012) and autoBUSTER (Bricogne G, Blanc E et al. 2011) with ligand restraints generated by Grade server (Global Phasing) or phenix.elbow (Moriarty, Grosse-Kunstleve et al. 2009). Protein geometry analysis revealed 0.63%, 0.55%, 0.94%, 0.72%, 1.02% Ramachandran outliers, with 95.43%, 95.27%, 94.68%, 93.99, 92.18% residues in favoured regions and 3.94%, 4.18%, 4.38%, 5.29%, 6.80% residues in allowed regions for the complexes with dBET6, 23, 55, 57 and 70 respectively.

The DDB1ΔB-CRBN-dBET57-BRD4_{BD1} complex crystallized in space group 1422 with a single complex in the unit cell. PHASER (McCoy, Grosse-Kunstleve et al. 2007) was used for molecular replacement using models of hsDDB1ΔB-hsCRBN-HBD derived from pdb: 5fqd, hsCRBN-NTD derived from pdb: 5fqd, and BRD4_{BD1} (pdb: 3mxf). The model was rigid body refined using PHENIX.REFINE (Afonine, Grosse-Kunstleve et al. 2012) and the hsCRBN-CTD was subsequently placed using Coot jiggle fit (part of Coot EM scripts from Alan Brown and Paul Emsley). The final model was rigid body refined using PHENIX.REFINE and autoBUSTER (Bricogne G, Blanc E et al. 2011). Anomalous maps were calculated with PHENIX.MAPS (Afonine, Grosse-Kunstleve et al. 2012).

Figures were generated with PyMOL (The PyMOL Molecular Graphics System, Version 1.8.6.0 Schrödinger, LLC) and model quality was assessed with MOLPROBITY (Chen, Arendall et al. 2010). Interaction surfaces were determined with PISA (Krissinel and Henrick 2007). The IKZF1 homology model was taken from (Petzold, Fischer et al. 2016).

### Example 13: Time-Resolved Fluorescence Resonance Energy Transfer (TR-FRET)

Compounds in dimerization assays were dispensed in a 384-well microplate (Corning, 4514) using D300e Digital Dispenser (HP) normalized to 2% DMSO into 200 nM biotinylated His₆-avi-bromodomain (WT or mutant) or 80 nM biotinylated StrepII-avi-IKZF1Δ, 100 nM His₆-DDB1ΔB-His₆-CRBN_{BODIPY-Spycatcher} and 2 nM terbium-coupled streptavidin (Invitrogen) in a buffer containing 50 mM Tris pH 7.5, 100 mM NaCl, 0.1% Pluronic F-68 solution (Sigma) and 2% DMSO (4% DMSO final). Compounds in CRBN mutants dimerization assay were dispensed as described above into 200 nM His₆-DDB1-His₆-CRBNₘᵤₜₐₙₜₛ or 200 nM His₆-DDB1ΔB-His₆-CRBN_{WT}, 100 nM BRD4_{BD1-BODIPY-SpyCatcher} and 2 nM terbium-anti-HIS Ab (Invitrogen) in a buffer containing 50 mM Tris pH 7.5, 100 mM NaCl, 0.1% Pluronic F-68 solution (Sigma) and 2% DMSO (4% DMSO final). Before TR-FRET measurements were conducted, the reactions were incubated for 15 min at RT. After excitation of terbium fluorescence at 337 nm, emission at 490 nm (terbium) and 520 nm (BODIPY) were recorded with a 70 μs delay over 600 μs to reduce background fluorescence and the reaction was followed over 30 200 second cycles of each data point using a PHERAstar FS microplate reader (BMG Labtech). The TR-FRET signal of each data point was extracted by calculating the 520/490 nm ratio. The heterobifunctional nature of small molecule degraders results in a three-body binding equilibrium complicated by potential cooperativity or avidity effects arising from protein-protein interactions (Douglass, Miller et al. 2013), all of which precludes direct interpretation of the binding data. However, assuming constant concentrations of BRD4_{BD1}, DDB1ΔB-CRBN, and fluorescent labels, as well as similar binding conformations, the peak height of the TR-FRET can be used as an indication for the amount of tertiary complex formation (containing BRD4_{BD1/BD2}, dBET, and CRBN) (Douglass, Miller et al. 2013). The peak height of TR-FRET dBET dose response data was calculated in GraphPad Prism 7 using Area Under Curve analysis for three independent replicates (n=3) and the mean peak height and standard deviation calculated.

Counter titrations with unlabelled proteins were carried out by addition of solution of 200 nM His₆-DDB1ΔB-His₆-CRBN_{BODIPY-Spycatcher}, 160 nM biotinylated His₆-Avi-IKZF1 Å, 4 nM terbium-coupled streptavidin and 2 μM of dBET57, incubated for 15 min on ice, to equal volume of titrated unlabelled His₆-Avi-BRD4_{BD1} or His₆-Avi-BRD4_{BD2} to the final assay concentrations.

The 520/490 nm ratios in IKZF1Δ TR-FRET assays were plotted to calculate the half maximal effective concentrations (EC₅₀—for unlabelled protein titrations) or IC₅₀ (for compound titrations) assuming a single binding site using GraphPad Prism 7 variable slope equation. The standard deviation in IKZF1A TR-FRET compound titrations was calculated from three biological replicates (n=3) as an average of 5 technical replicates per well per experiment, or as an average of 5 technical replicates of single experiment for unlabelled protein titrations.

### Example 14: Fluorescence Polarization

Atto565-conjugated lenalidomide (10 nM) was mixed with increasing concentration of purified his₆-DDB1ΔB-his₆-CRBN (10 μM final top concentration, 2-fold, 23 point dilution and DMSO control) in 384-well microplates (Corning, 4514) and incubated for 15 min at RT. The change in fluorescence polarization was monitored using a PHERAstar FS microplate reader (BMG Labtech) for 20 min in 120 s cycles. The Atto565-lenalidomide bound fraction was calculated as described (Marks, Qadir et al. 2005) and the K_{d} was obtained from a fit in GraphPad Prism 7 from four independent replicates (n=4).

Compounds in Atto565-Lenalidomide displacement assay were dispensed in a 384-well microplate (Corning, 4514) using D300e Digital Dispenser (HP) normalized to 2% DMSO into 10 nM Atto565-Leanlidomide, 100 nM DDB1ΔB-CRBN, 50 mM Tris pH 7.5, 100 mM NaCl, 0.1% Pluronic F-68 solution (Sigma), 0.5 mg/ml BSA (Sigma) containing 2% DMSO (4% DMSO final). Compound titrations were performed in presence of 0, 1, 5, 20 μM of unbiotinylated his₆-avi-BRD4_{BD1} or his₆-avi-BRD4_{BD2} and incubated for 60 min at RT. The change in fluorescence polarization was monitored using a PHERAstar FS microplate reader (BMG Labtech) for 20 min in 200 s cycles. Data from two independent measurements (n=2) was plotted and IC₅₀ values estimated using variable slope equation in GraphPad Prism 7.

### Example 15: Cellular Degradation Assays

IKZF1Δ, BRD2_{BD1}, BRD2_{BD2}, BRD3_{BD1}, BRD3_{BD2}, BRD4_{BD1}, and BRD4_{BD2} were subcloned into mammalian pcDNA5/FRT Vector (Ampicillin and Hygromycin B resistant) modified to contain MCS-eGFP-P2A-mCherry. Stable cell lines expressing eGFP-protein fusion and mCherry reporter were generated using Flip-In 293 system. Plasmid (0.3 μg) and pOG44 (4.7 μg) DNA were preincubated in 100 μL of Opti-MEM I (Gibco, Life Technologies) media containing 0.05 mg/ml Lipofectamine 2000 (Invitrogen) for 20 min and added to Flip-In 293 cells containing 1.9 ml of DMEM media (Gibco, Life Technologies) per well in a 6-well plate format (Falcon, 353046). Cells were propagated after 48 h and transferred into a 10 cm² plate (Corning, 430165) in DMEM media containing 50 μg/ml of Hygromycin B (REF 10687010, Invitrogen) as a selection marker. Following 2-3 passage cycle FACS (FACSAria II, BD) was used to enrich for cells expressing eGFP and mCherry.

Cells were seeded at 30-50% confluency in either 24, 48 or 96 well plates (3524, 3548, 3596 respectively, Costar) a day before compound treatment. Titrated compounds were incubated with cells for 5 h following trypsinisation and resuspention in DMEM media, transferred into 96-well plates (353910, Falcon) and analyzed by flow cytometer (guava easyCyte HT, Millipore). Signal from 5000 cells per well was acquired in singlicate or duplicate and the eGFP and mCherry florescence monitored. Data was analyzed using FlowJo (FlowJo, LCC). Forward and side scatter outliers, frequently associated with cell debris, were removed leaving >90% of total cells, followed by removal of eGFP and mCherry signal outliers, leaving 88-90% of total cells creating the set used for quantification. The eGFP protein abundance relative to mCherry was then quantified as a ten-fold amplified ratio for each individual cell using the formula: 10×eGFP/mCherry. The median of the ratio was then calculated per set, normalized to the median of the DMSO ratio, and is denoted as relative abundance. Standard deviation is calculated from four replicates (n=4) unless described otherwise.

### Example 16: Western Blot for Cellular BRD2/3/4 Degradation

HEK293T cells were seeded at 90% confluency in 12 well plates (353043, Falcon), left to attach for 1.5 h, followed by the compound treatment for 5 h. Primary and secondary antibodies used included anti-BRD4 at 1:1000 dilution (A301-985A-M, Bethyl Laboratories), anti-BRD2 at 1:2,000 dilution (A302-582A, Bethyl Laboratories), anti-BRD3 at 1:500 dilution (ab56342, Abcam), anti-GAPDH at 1:10,000 dilution (G8795, Sigma), IRDye680 Donkey anti-mouse at 1:10,000 dilution (926-68072, LiCor) and IRDye800 Goat anti-rabbit at 1:10,000 dilution (926-32211, LiCor).

### Example 17: Sample Preparation TMT LC-MS3 Mass Spectrometry

MM.1s cell were treated with DMSO, 1 μM dBET23, or dBET70 in biological triplicates for 5 hours and cells harvested by centrifugation. Lysis buffer (8 M Urea, 1% SDS, 50 mM Tris pH 8.5, Protease and Phosphatase inhibitors from Roche) was added to the cell pellets to achieve a cell lysate with a protein concentration between 2-8 mg mL⁻¹. A micro-BCA assay (Pierce) was used to determine the final protein concentration in the cell lysate. 200 μg proteins for each sample were reduced and alkylated as previously described. Proteins were precipitated using methanol/chloroform. In brief, four volumes of methanol were added to the cell lysate, followed by one volume of chloroform, and finally three volumes of water. The mixture was vortexed and centrifuged to separate the chloroform phase from the aqueous phase. The precipitated protein was washed with one volume of ice-cold methanol. The washed precipitated protein was allowed to air dry. Precipitated protein was resuspended in 4 M Urea, 50 mM Tris pH 8.5. Proteins were first digested with LysC (1:50; enzyme:protein) for 12 hours at 25° C. The LysC digestion was diluted down in 1 M Urea, 50 mM Tris pH 8.5 and then digested with trypsin (1:100; enzyme:protein) for another 8 hours at 25° C. Peptides were desalted using a Cis solid phase extraction cartridges (Waters). Dried peptides were resuspended in 200 mM EPPS, pH 8.0. Peptide quantification was performed using the micro-BCA assay (Pierce). The same amount of peptide from each condition was labelled with tandem mass tag (TMT) reagent (1:4; peptide:TMT label) (Pierce). The 10-plex labelling reactions were performed for 2 hours at 25° C. Modification of tyrosine residue with TMT was reversed by the addition of 5% hydroxyl amine for 15 minutes at 25° C. The reaction was quenched with 0.5% TFA and samples were combined at a 1:1:1:1:1:1:1:1:1:1 ratio. Combined samples were desalted and offline fractionated into 96 fractions using an aeris peptide xb-c18 column (phenomenex) at pH 8.0. Fractions were recombined in a non-continuous manner into 24 fractions and every second fraction was used for subsequent mass spectrometry analysis.

Data were collected using an Orbitrap Fusion Lumos mass spectrometer (Thermo Fisher Scientific, San Jose, Calif., USA) coupled with a Proxeon EASY-nLC 1200 LC pump (Thermo Fisher Scientific). Peptides were separated on a 75 μm inner diameter microcapillary column packed with 35 cm of Accucore C18 resin (2.6 pin, 100 Å, ThermoFisher Scientific). Peptides were separated using a 3 hr gradient of 6-27% acetonitrile in 0.125% formic acid with a flow rate of 400 nL/min.

Each analysis used an MS³-based TMT method as described previously (McAlister, Nusinow et al. 2014). The data were acquired using a mass range of m/z 350-1350, resolution 120,000, AGC target 1×10⁶, maximum injection time 100 ms, dynamic exclusion of 120 seconds for the peptide measurements in the Orbitrap. Data dependent MS² spectra were acquired in the ion trap with a normalized collision energy (NCE) set at 35%, AGC target set to 1.8×10⁴ and a maximum injection time of 120 ms. MS³ scans were acquired in the Orbitrap with a HCD collision energy set to 55%, AGC target set to 1.5×10⁵, maximum injection time of 150 ms, resolution at 50,000 and with a maximum synchronous precursor selection (SPS) precursors set to 10.

### Example 18: LC-MS Data Analysis

Proteome Discoverer 2.1 (Thermo Fisher) was used to for .RAW file processing and controlling peptide and protein level false discovery rates, assembling proteins from peptides, and protein quantification from peptides. MS/MS spectra were searched against a Uniprot human database (September 2016) with both the forward and reverse sequences. Database search criteria are as follows: tryptic with two missed cleavages, a precursor mass tolerance of 50 ppm, fragment ion mass tolerance of 1.0 Da, static alkylation of cysteine (57.02146 Da), static TMT labelling of lysine residues and N-termini of peptides (229.16293 Da), and variable oxidation of methionine (15.99491 Da). TMT reporter ion intensities were measured using a 0.003 Da window around the theoretical m/z for each reporter ion in the MS³ scan. Peptide spectral matches with poor quality MS³ spectra were excluded from quantitation (<summed signal-to-noise across 10 channels and <0.5 precursor isolation specificity).

Reporter ion intensities were normalised and scaled in the R framework (Team 2013). Statistical analysis was carried out using the limma package within the R framework (Ritchie, Phipson et al. 2015).

### Example 19: Protein Docking

All protein docking was carried out using Rosetta 3.7 provided through SBGrid (Morin, Eisenbraun et al. 2013). Input models were downloaded from the PDB (hsCRBN pdb: 4tz4; BRD4_{BD1} pdb: 3mxf, BRD4_{BD2} pdb: 2ouo, and hsCSNK1A1 pdb: 5fqd). Ligand conformers were generated using OpenEye Omega (OpenEye scientific) and parameter files generated using Rosetta ‘molfile_to_params.py’. Relevant PDB's were combined into a single file and prepared for docking using the Rosetta ‘docking_prepack_protocol’ program. Initial global docking was performed using Rosetta ‘docking_protocol_mpi’ with the following command line options:

partners A_B—dock_pert 5 25—randomize2—ex1 ex2aro-nstruct 20000 providing the combined pdb and ligand specific parameter files as input.

For Ck1α, and the initial analysis of BRD4_{BD1}, the two lowest scoring solutions were used for local perturbation docking with Rosetta ‘docking_protocol_mpi’ with the following command line options:

partners A_B—dock_pert 8 18—ex1 ex2aro-nstruct 2000

To assess the landscape of possible binding modes for BRD4_{BD1} and BRD4_{BD2}, the top 200 lowest scoring docking decoys were selected and hierarchical clustered according to the compound centroids and orientations. The lowest scoring model of each cluster was loaded into pymol and decoys that would position the thalidomide and JQ1 binding sites on CRBN and BRD4_{BD1/2}, respectively, more than 30 Å apart. The remaining decoys were considered.

Methods were developed for the design of heterobifunctional compounds based on computational protein-protein docking, including methods for analysis of the docking results and the inference of design information for chemical synthesis. These methods were applied to the BET family protein BRD4 to synthesize working examples.

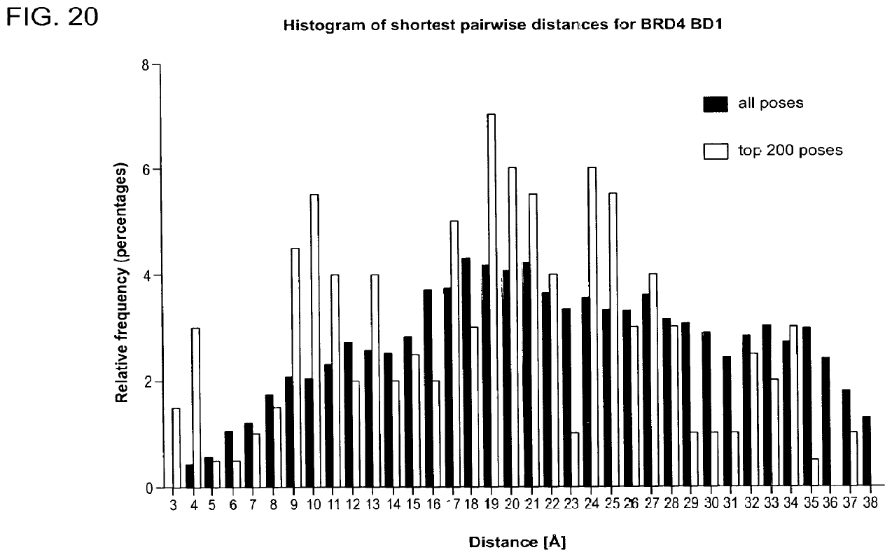

Protein-protein docking programs such as Rosetta output docked poses of the two proteins. In one embodiment, BRD4_{BD1} was docked with CRBN in the presence of the ligands, JQ1 and lenalidomide respectively, resulting in 10,000 scored poses. Then, the shortest distance paths between a set of solvent exposed atoms on both ligands was calculated and plotted those as a histogram of the distances (FIG. 20). Histogram of 10,000 distances and the distances from top 200 scoring poses present clearly distinct profiles. The profile of all poses approximates a normal distribution, whereas the profile of the top 200 poses has clear regions (i.e., clusters) of distances that occurred with higher frequency (FIG. 20). These clusters indicate a preference for the complex formation in these particular distance constraints.

Data analysis and statistics for all steps were performed using the R framework (Team 2013) or Matlab.

### Example 20: In Silico Docking to Design Degrader Molecules

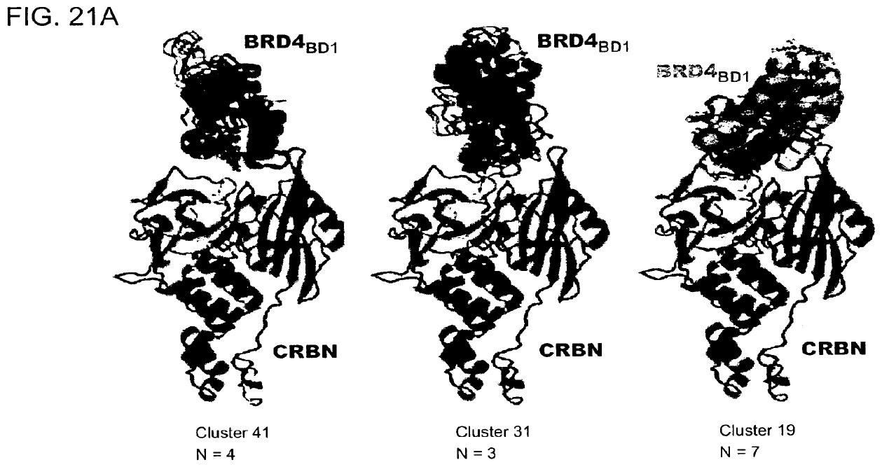

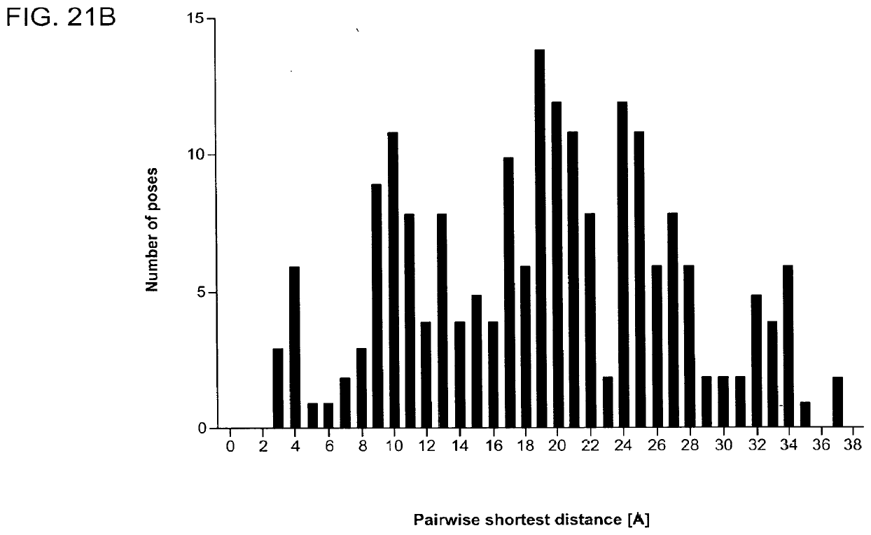

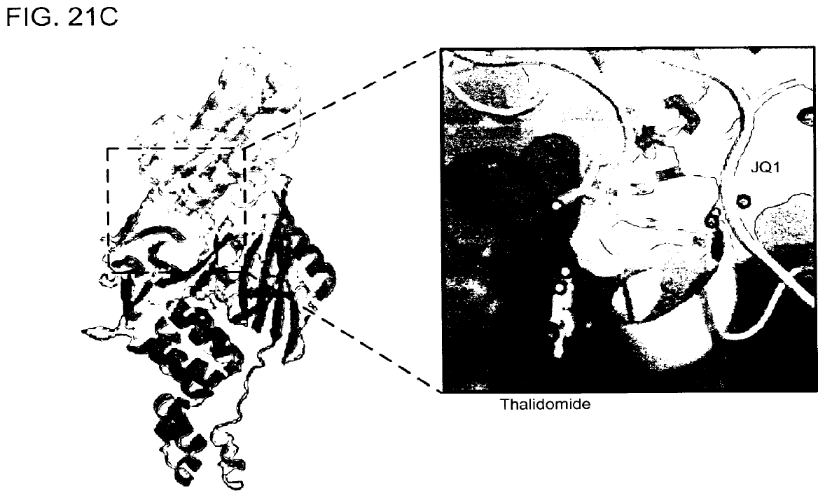

FIG. 21A-FIG. 21B is a series of schematic diagrams and a graph showing in silico docking to design degrader molecules using the shortest distance (i.e., Euclidian distance) algorithm. FIG. 21A is a cartoon showing representations for representative clusters obtained by k-means clustering of the top 200 global docking poses between CRBN (pdb: 4tz4) and BRD4_{BD1} (pdb: 3mxf). FIG. 21B is a histogram of the pairwise shortest distances for the top 200 docking poses. FIG. 21C is a schematic showing a close-up view on the proximity of the JQ1 thiophene and lenalidomide that provided the rationale for synthesizing ZXH-2-147 and ZXH-3-26. Atoms used for calculation of the pairwise shortest distances between JQ1 and lenalidomide are highlighted in black circles.

Protein Docking

All protein docking was carried out using Rosetta 3.7 provided through SBGrid (Morin, Eisenbraun et al. 2013). Input models were downloaded from the PDB (hsCRBN pdb: 4tz4; BRD4_{BD1} pdb: 3mxf, BRD4_{BD2} pdb: 2ouo, and hsCSNK1A1 pdb: 5fqd). Ligand conformers were generated using OpenEye Omega (OpenEye scientific) and parameter files generated using Rosetta ‘molfile_to_params.py’. Relevant pdb structure coordinates were combined into a single file and prepared for docking using the Rosetta ‘docking_prepack_protocol’ program. Initial global docking was performed using Rosetta ‘docking_protocol_mpi’ with the following command line options:
partners A_B—dockpert 5 25—randomize2—ex1 ex2aro-nstruct 20000
   providing the combined pdb and ligand specific parameter files as input.

For Ck1α, and the initial analysis of BRD4_{BD1}, the two lowest scoring solutions were used for local perturbation docking with Rosetta ‘docking_protocol_mpi’ with the following command line options:
partners A_B—dockpert 8 18—ex1 ex2aro-nstruct 2000

To assess the landscape of possible binding modes for BRD4_{BD1} and BRD4_{BD2}, the top 200 lowest scoring docking decoys were selected and hierarchical clustered according to the compound centroids and orientations.

The shortest pairwise distance between selected set of atoms on JQ1 and set of atoms on lenalidomide (see highlighted atoms in FIG. 21C) was calculated in Pymol (The PyMOL Molecular Graphics System, Version 1.8.6.0 Schrödinger, LLC) as Euclidean distance for each of the top 200 poses. The histogram was obtained in GraphPad Prism 7 using Column Analysis—Frequency Distribution.

### Example 21: Plasticity in Binding Confers Selectivity in Ligand Induced Protein Degradation

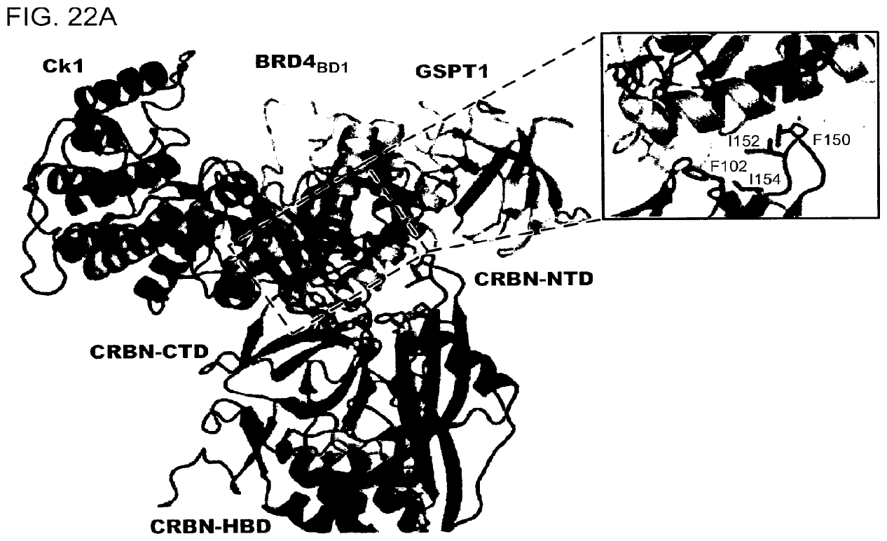

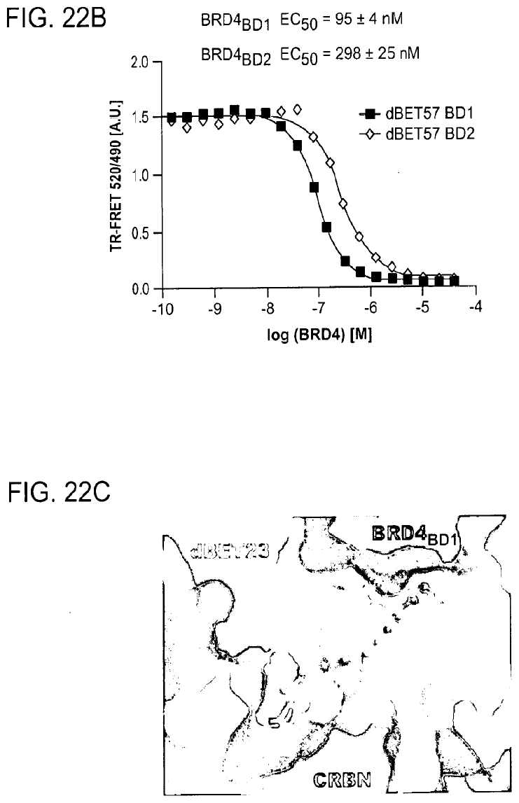

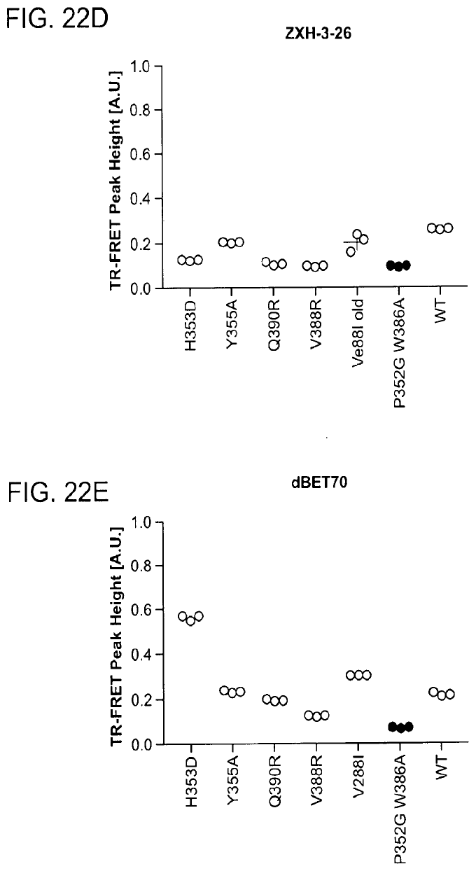

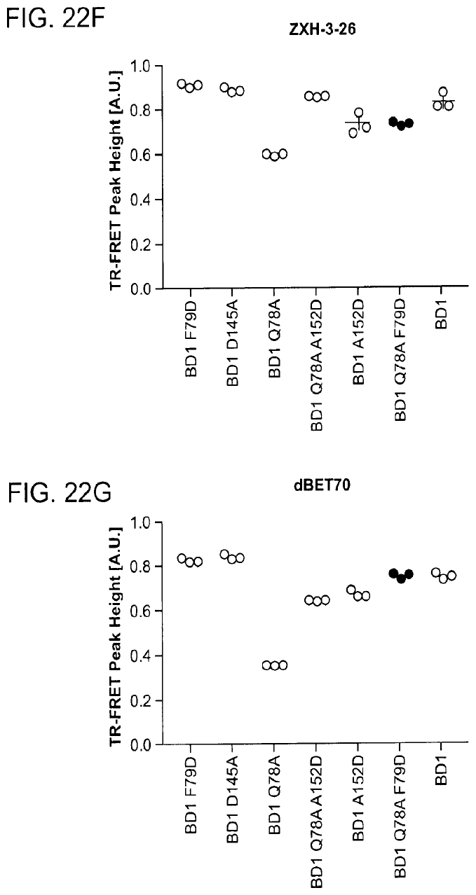

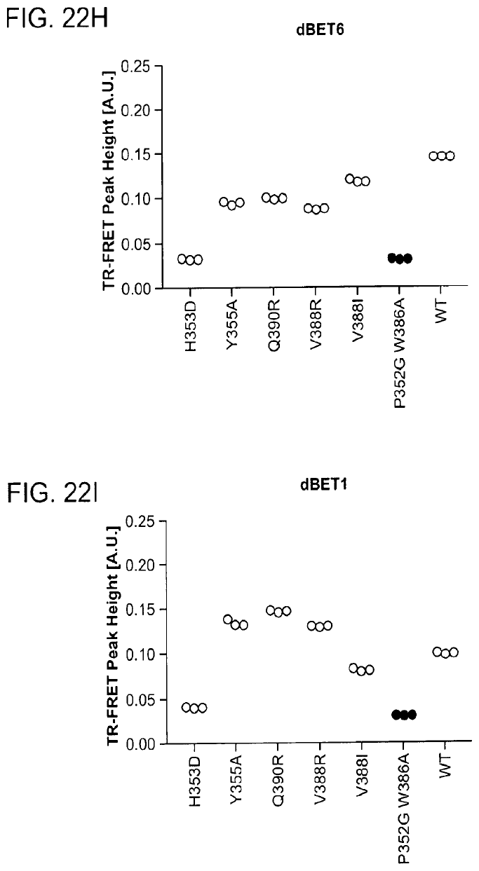

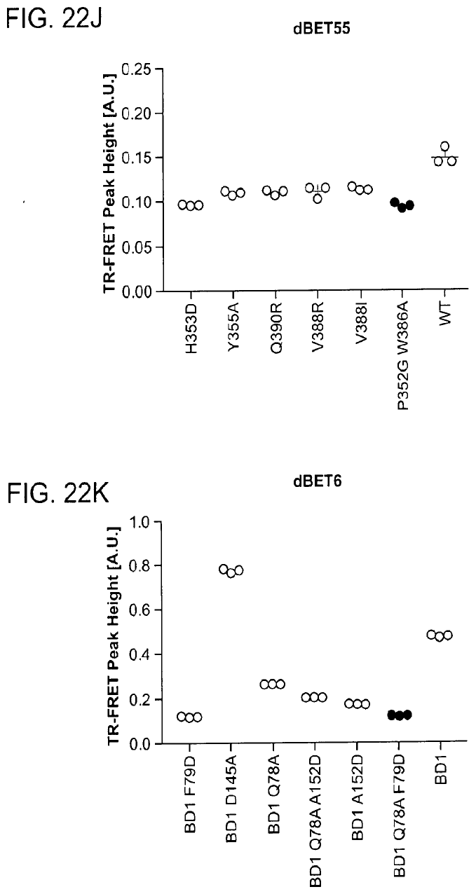

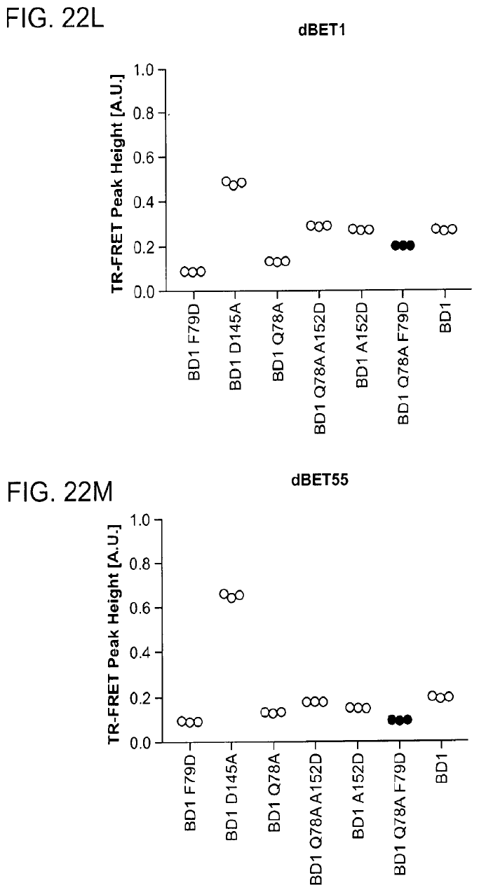

FIG. 22A-FIG. 22M is a series of graphs showing plasticity of CRBN-substrate interactions. As described herein, plasticity in binding confers selectivity in ligand induced protein degradation. Specifically, FIG. 22A-FIG. 22M show additional mutation data for ZXH-3-26 and dBET70 confirming distinct BRD4_{BD1} binding modes that these two molecules support. FIG. 22A is a schematic showing that CRBN utilizes different surfaces to interact with a variety with neo-substrates as illustrated by the superposition of DDB1ΔB-CRBN-dBET23-BRD4_{BD1}, DDB1ΔB-CRBN-lenalidomide-Ck1α (pdb: 5fqd), and DDB1-CRBN-CC885-GSPT1 (pdb: 5hxb). Top right, close-up of the common hydrophobic interface between GSPT1-CRBN-NTD and BRD4_{BD1}-CRBN-NTD. FIG. 22B is a line graph showing that the structures of DDB1-CRBN-dBET23-BRD4_{BD1} and DDB1-CRBN-lenalidomide-CK1a suggest mutually exclusive binding of BRD4 with neo-substrates such as Ck1α or IKZF1/3, which is confirmed by titrating BRD4_{BD1} or BRD4_{BD2} into a preformed complex of DDB1-CRBN-dBET57-IKZF1Δ. Data is presented as mean and standard deviation of 10 technical replicates of a single experiment (n=1). FIG. 22C is a schematic showing the surface representation of CRBN and BRD4_{BD1} of DDB1-CRBN-dBET23-BRD4_{BD1} crystal structure, showing dBET23 as stick representation. The hypothetical linker path from the acid position on JQ1 is shown with red spheres indicating the distance of a carbon-carbon bond and illustrating that the 2-carbon linker of dBET57 would be insufficient to bridge the gap. FIG. 22D is a graph showing TR-FRET. ZXH-3-26 degrader titrated to BRD4_{BD1-SPYCATCHER-BODIPY} and Terbium-antiHis antibody, and wild type or various mutants of His6-DDB1-His6-CRBN complex. The peak height of the dose response curve for three independent replicates was quantified and is depicted as dot-plot. TR-FRET data in this figure are independent replicates presented as means±s.d. (n=3). (FIG. 22E, FIG. 22H, FIG. 22I, and FIG. 22J) as in FIG. 22D, but for dBET70, dBET6, dBET and dBET55, respectively. FIG. 22F is a graph showing TR-FRET. ZXH-3-26 degrader titrated to DDB1ΔB-CRBN_{SPYCATCHER-BODIPY}, Terbium-Streptavidin and wild type or mutants of BRD4_{BD1-biotin}. The peak height of the dose response curve for three independent replicates was quantified and is depicted as dot-plot. TR-FRET data in this figure are presented as means±s.d. (n=3). FIG. 22G, FIG. 22K, FIG. 22L, and FIG. 22M as in FIG. 22F, but for dBET70, dBET6, dBET and dBET55, respectively.

### Example 22: Experimental Validation of DDB1-CRBN-dBET57-BRD4_{BD1} Structure

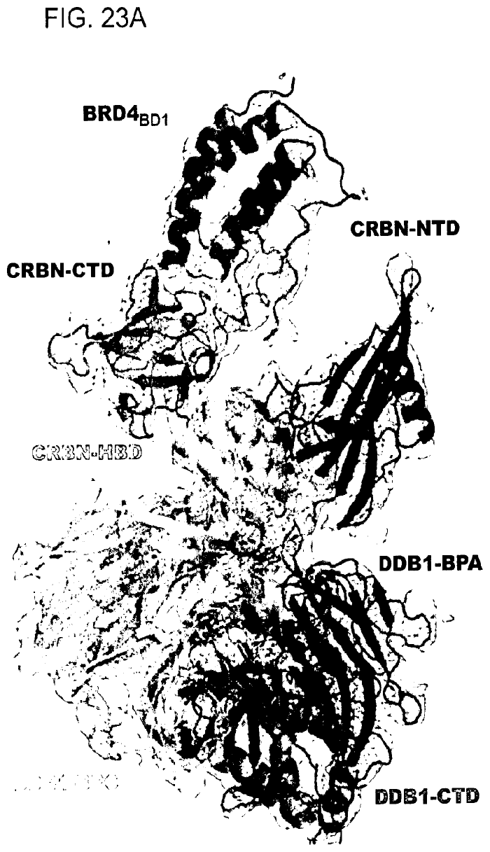

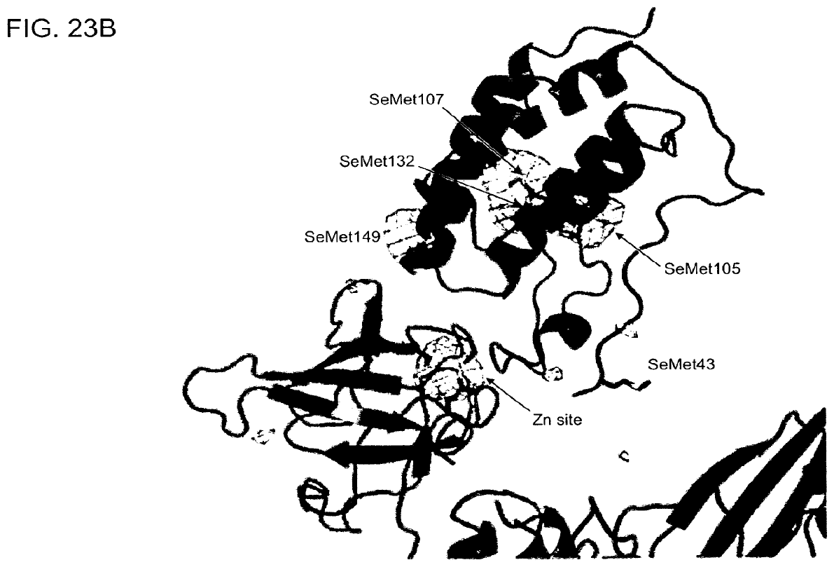

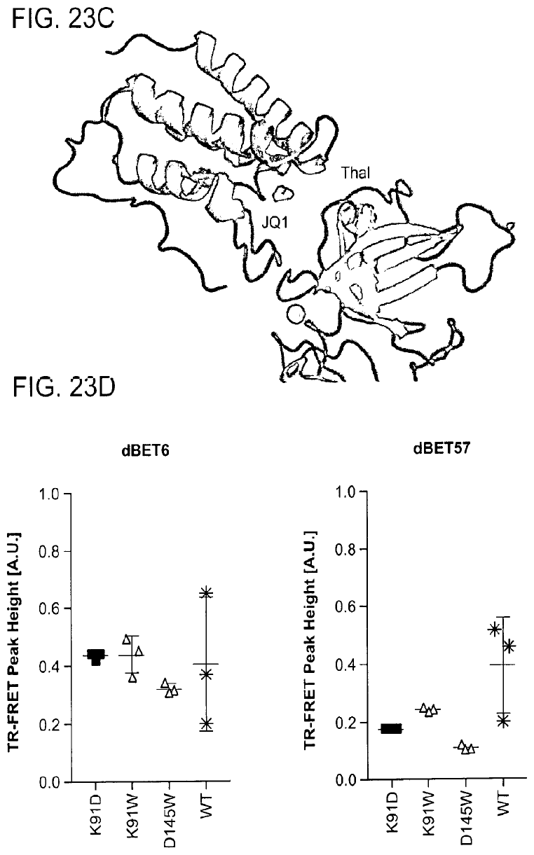

FIG. 23A-FIG. 23D is a series of schematics and graphs showing the experimental validation of DDB1-CRBN-dBET57-BRD4_{BD1} structure. Specifically, FIG. 23A-FIG. 23D show further validation of dBET57 binding mode with TR-FRET assays. FIG. 23A is a cartoon representation of DDB1-CRBN-dBET57-BRD4_{BD1} complex with the 2F_{O}-F_{C} map contoured at 1.5 σ. Domains are colored as DDB1-BPA (red), DDB1-BPC (orange), DDB1-CTD (grey), CRBN-NTD (blue), CRBN-HBD (cyan), CRBN-CTD (green), and BRD4_{BD1} (magenta). CRBN was found in a not-previously-observed conformation, in which the thalidomide binding CRBN-CTD domain translates and rotates away from the CRBN-HBD and CRBN-NTD domains. This results in an open conformation that exposes large areas of CRBN that are typically buried. The high salt crystallization condition could be a driver of this structural rearrangement, and together with crystal contacts induce this conformation. However, it cannot be excluded that this conformational dynamic is an intrinsic feature of CRBN to accommodate a variety of substrates and future studies are necessary to address this. Based on the compatibility of the observed BRD4_{BD1} binding conformation with the open and closed CRBN conformations, for the interpretation of the data the conformational change is negligible. FIG. 23B is a cartoon representation of DDB1-CRBN-dBET57-SeMetBRD4_{BD1} complex. Anomalous difference map contoured at 3 σ shown in orange for data collected at the Se peak showing the position of the Se atoms and Zn. FIG. 23C is a schematic showing an F_{O}-F_{C} map of native DDB1-CRBN-dBET57-BRD4_{BD1} contoured at 3.0 σ and shown in green, carved around the JQ1 and thalidomide sites. Positive difference density is observed for the Thalidomide (Thal) and JQ1 binding sites. FIG. 23D is a graph showing TR-FRET, dBET6 or dBET57 degrader titrated to DDB1ΔB-CRBN_{SPYCATCHER-BODIPY}, Terbium-Streptavidin and wild type or mutants of BRD4_{BD1-biotin}. The peak height of the dose response curve for three independent replicates was quantified and is depicted as dot-plot. TR-FRET data in this figure are independent replicates presented as means±s.d. (n=3).

### Example 23: Selective Degradation of BRD4

FIG. 24A-FIG. 24L is a series of graphs showing selective degradation of BRD4. Specifically, FIG. 24A-FIG. 24L show how family wide protein sequence alignment is used to highlight protein hotspots. Poses where these hotspots are present in the E3 ligase-target/protein interface (e.g., FIG. 24K-Q84) can be selectively targeted with heterobifunctional molecules and can result in family wide selective complex formation and resulting degradation. FIG. 24A is a graph showing the quantitative assessment of cellular degradation using EGFP/mCherry reporter assay. Cells stably expressing BRD4_{BD1}-EGFP (or constructs harbouring BRD2_{BD1}, BRD2_{BD2}, BRD3_{BD1}, BRD3_{BD2}, BRD4_{BD2}) and mCherry were treated with increasing concentrations of ZXH-2-147 and the EGFP and mCherry signals followed using flow cytometry analysis. FIG. 24B is the same as in FIG. 24A, but for ZXH-2-184. FIG. 24C is the same as FIG. 24A, but for ZXH-3-27. Data in a-c are singlicate experiments (n=1). FIG. 24D is a graph showing TR-FRET, dBET degrader titrated to DDB1ΔB-CRBN_{SPYCATCHER-BODIPY}, Terbium-Streptavidin and wild type or mutants of BRD4_{BD1-biotin}. The peak height of the dose response curve for three independent replicates was quantified and is depicted as dot-plot. TR-FRET data in this figure are presented as means±s.d. (n=3). FIG. 24E, FIG. 24F, FIG. 24G, FIG. 24H, FIG. 24I, FIG. 24J is as in FIG. 24D, but for dBET6, dBET23, dBET55, dBET57, dBET70 and ZXH-3-26 respectively. Interestingly, mutation of Q84 to R (as in BRD2 or K as in BRD3, see FIG. 24D-FIG. 24J) decreases complex formation with CRBN mediated by ZXH-3-26 (FIG. 24D) reference to WT, consistent with observed specificity for BRD2/3. FIG. 24K is a cartoon representation of docking pose from cluster 19 (see, FIG. 21A-FIG. 21C) serving as a rationale for design of ZXH-3-26. BRD4_{BD1} shown in green and CRBN in blue. Highlighted residues of BRD4 different between BRD2/3. Residue Q84 (R in BRD2, Y in BRD3) highlighted in orange. FIG. 24L is a sequence alignment of first bromodomain of BRD2, BRD3, BRD4 and BRDT. Highlighted residues of BRD4 different between BRD2/3. Residue Q84 (R in BRD2, Y in BRD3) highlighted with an arrow.

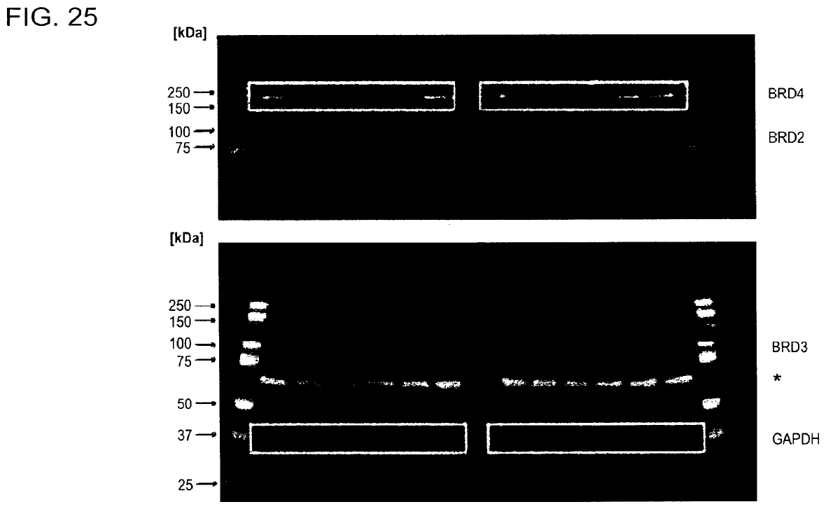

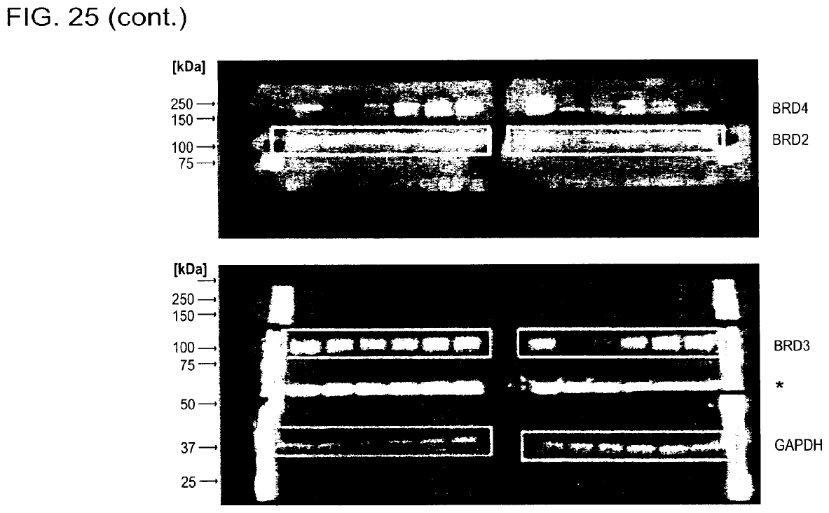

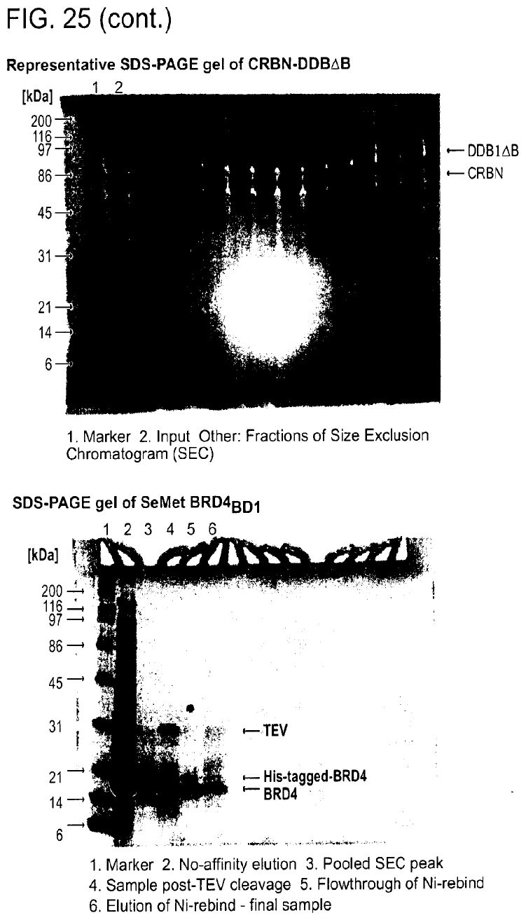

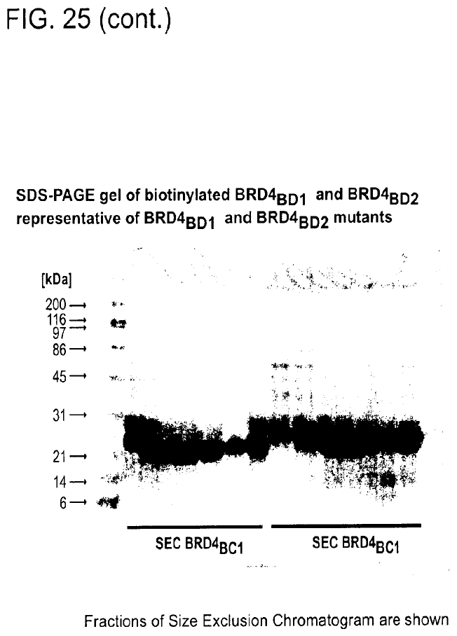

FIG. 25 is a series of uncropped immunoblots, which support the data presented above. Boxed areas correspond to image regions represented in the indicated main text and Supplementary figures. Western blots have been flipped vertically to represent increasing concentrations of Compound. SDS-PAGE gel images for representative preparations of DDBΔB-CRBN, SeMet-BRD4_{BD1}, biotinylated BRD4_{BD1} and biotinylated BRD4_{BD2} are shown.

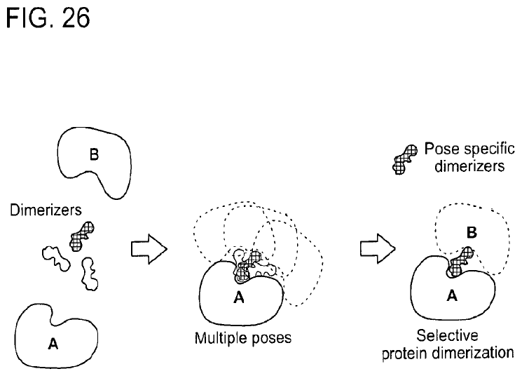

FIG. 26 is a schematic showing a graphical overview of some of the methods described herein. Multiple suitable dimerizers can induce dimerization of two proteins A and B resulting in multiple A-dimerizer-B ternary complex poses. Finally, dimerizers can be developed to explore a specific pose, leading to selective protein dimerization and/or degradation.

The tables are set forth below.

TABLE 1
Data collection and refinement statistics.

| | | | | | DDB1ΔB-CRBN- | |
|---|---|---|---|---|---|---|
| | DDB1ΔB-CRBN- | | DDB1ΔB-CRBN- | | dBET55-BRD4_{BD1} | |
| | dBET6-BRD4_{BD1} | | dBET23-BRD4_{BD1} | | D145A | |
| Data collection | | | | | | |
| Space group | P 6₅ 2 2 | | P 6₅ 2 2 | | P 6₅ 2 2 | |
| Cell dimensions | | | | | | |
| a, b, c (Å) | 115.40, 115.40, 588.14 | | 115.57, 115.57, 596.32 | | 115.20, 115.20, 597.14 | |
| α, β, γ (°) | 90, 90, 120 | | 90, 90, 120 | | 90, 90, 120 | |
| Resolution (Å) | 49.79-3.33 | (3.49-3.33) | 49.87-3.49 | (3.68-3.49) | 149.28-3.99 | (4.31-3.99) |
| R_{merge} | 0.179 | (5.471) | 0.128 | (3.561) | 0.280 | (2.227) |
| Rₚᵢₙ | 0,032 | (0.978) | 0.041 | (1.173) | 0.072 | (0.582) |
| CC_{1/2} | 1.000 | (0.469) | 0.999 | (0.328) | 0.991 | (0.452) |
| I/σI | 16.4 | (0.9) | 11.4 | (0.7) | 7.69 | (1.1) |
| Completeness (%) | 100.0 | (100.0) | 99.5 | (97.3) | 100.0 | (100.0) |
| Redundancy | 32.2 | (33.2) | 11.4 | (10.5) | 17.0 | (16.1) |
| Refinement | | | | | | |
| Resolution (Å) | 49.79-3.33 | (3.45-3.33) | 49.35-3.50 | (3.63-3.50) | 99.77-3.99 | (4.13-3.99) |
| No. reflections | 35251 | (3287) | 30453 | (2671) | 21193 | (2038) |
| R_{work} | 0.1994 | (0.3605) | 0.2123 | (0.3551) | 0.2886 | (0.3757) |
| R_{free} | 0.2344 | (0.4380) | 0.2555 | (0.3848) | 0.3334 | (0.3912) |
| No. atoms | 10373 | | 10331 | | 10291 | |
| Protein | 10313 | | 10268 | | 10290 | |
| Ligand/ion | 60 | | 63 | | 1 | |
| Water | 0 | | 0 | | 0 | |
| B-factors | 175.87 | | 204.01 | | 189.70 | |
| Protein | 176.08 | | 204.26 | | 189.70 | |
| Ligand/ion | 140.19 | | 162.21 | | 133.84 | |
| Water | — | | — | | — | |
| R.m.s. deviations | | | | | | |

| | | | |
|---|---|---|---|
| Bond lengths (Å) | 0.002 | 0.007 | 0.002 |
| Bond angles (°) | 0.51 | 0.48 | 0.46 |

*Each dataset was collected from one crystal.
*Values in parentheses are for highest-resolution shell.

TABLE 2
Data collection and refinement statistics.

| | |
|---|---|
| DDB1ΔB-CRBN- | DDB1ΔB-CRBN- |
| dBET57-SeMetBRD4_{BD1} | dBET70-BRD4_{BD1} |

Data collection

| | | |
|---|---|---|
| Space group | I 4 2 2 | P 6₅ 2 2 |

Cell dimensions

| | | |
|---|---|---|
| a, b, c (Å) | 313.36, 313.36, 167.37 | 117.60, 117.60, 597.16 |
| α, β, γ (°) | 90, 90, 90 | 90, 90, 120 |

| Resolution (Å) | 147.60-6.34 | (7.08-6.34) | 149.29-4.38 | (4.90-4.38) |
|---|---|---|---|---|
| R_{merge} | 0.165 | (2.952) | 0.349 | (3.276) |
| Rₚᵢₘ | 0.036 | (0.593) | 0.059 | (0.548) |
| CC_{1/2} | 1.000 | (0.627) | 1.000 | (0.768) |
| I/σI | 15.3 | (1.3) | 8.5 | (14) |
| Completeness (%) | 98.1 | (93.4) | 99.7 | (99.8) |
| Redundancy | 25.4 | (26.2) | 36.9 | (36.7) |
| Refinement | | | | |
| Resolution (Å) | 147.60-6.34 | (6.57-6.34) | 100.40-4.38 | (4.54-4.38) |
| No. reflections | 8964 | (743) | 16770 | (1588) |
| R_{work} | 0.3368 | (0.4151) | 0.2754 | (0.3827) |
| R_{free} | 0.3805 | (0.5110) | 0.3013 | (0.4689) |
| No. atoms | 10042 | | 10314 | |
| Protein | 10041 | | 10313 | |
| Ligand/ion | 1 | | 1 | |
| Water | 0 | | 0 | |
| B-factors | 484.99 | | 278.70 | |
| Protein | 485.00 | | 278.71 | |
| Ligand/ion | 465.40 | | 197.08 | |
| Water | — | | — | |
| R.m.s. deviations | | | | |

| | | |
|---|---|---|
| Bond lengths (Å) | 0.011 | 0.002 |
| Bond angles (°) | 1.48 | 0.469 |

*Each dataset was collected from one crystal.
*Values in parentheses are for highest-resolution shell.

### REFERENCES

Abdulrahman, W., M. Uhring, I. Kolb-Cheynel, J.-M. Gamier, D. Moras, N. Rochel, D. Busso and A. Poterszmnan (2009). “A set of baculovirus transfer vectors for screening of affinity tags and parallel expression strategies.” Analytical Biochemistry 385 (2): 383-385.
Afonine, P. V., R. W. Grosse-Kunstleve, N. Echols, J. J. Headd, N. W. Moriarty, M. Mustyakimov, T. C. Terwilliger, A. Urzhumtsev, P. H. Zwart and P. D. Adams (2012). “Towards automated crystallographic structure refinement with phenix.refine.” Acta Crystallographica Section D 68 (4): 352-367.
Bricogne G, Blanc E, Brandl M, Flensburg C, Keller P, Paciorek P, Roversi P, Sharff A, Smart O, Vonrhein C and W. T (2011). BUSTER version 2.10.2. Cambridge, United Kingdom, Global Phasing Ltd.
Buchdunger, E., C. L. Cioffi, N. Law, D. Stover, S. Ohno-Jones, B. J. Druker and N. B. Lydon (2000). “Abl protein-tyrosine kinase inhibitor STI571 inhibits in vitro signal transduction mediated by c-kit and platelet-derived growth factor receptors.” J Pharmacol Exp Ther 295 (1): 139-145.
Chau, N. G., S. Hurwitz, C. M. Mitchell, A. Aserlind, N. Grunfeld, L. Kaplan, P. Hsi, D. E. Bauer, C. S. Lathan, C. Rodriguez-Galindo, R. B. Tishler, R. I. Haddad, S. E. Sallan, J. E. Bradner and C. A. French (2016). “Intensive treatment and survival outcomes in NUT midline carcinoma of the head and neck.” Cancer 122 (23): 3632-3640.
Chen, V. B., W. B. Arendall, III, J. J. Headd, D. A. Keedy, R. M. Immormino, G. J. Kapral, L. W. Murray, J. S. Richardson and D. C. Richardson (2010). “MolProbity: all-atom structure validation for macromolecular crystallography.” Acta Crystallographica Section D 66 (1): 12-21.
Douglass, E. F., Jr., C. J. Miller, G. Sparer, H. Shapiro and D. A. Spiegel (2013). “A comprehensive mathematical model for three-body binding equilibria.” J Am Chem Soc 135 (16): 6092-6099.
Filippakopoulos, P., S. Picaud, M. Mangos, T. Keates, J. P. Lambert, D. Barsyte-Lovejoy, I. Felletar, R. Volkmer, S. Müller, T. Pawson, A. C. Gingras, C. H. Arrowsmith and S. Knapp (2012). “Histone recognition and large-scale structural analysis of the human bromodomain family.” Cell 149 (1): 214-231.
Filippakopoulos, P., J. Qi, S. Picaud, Y. Shen, W. B. Smith, O. Fedorov, E. M. Morse, T. Keates, T. T. Hickman, I. Felletar, M. Philpott, S. Munro, M. R. McKeown, Y. Wang, A. L. Christie, N. West, M. J. Cameron, B. Schwartz, T. D. Heightman, N. La Thangue, C. A. French, O. Wiest, A. L. Kung, S. Knapp and J. E. Bradner (2010). “Selective inhibition of BET bromodomains.” Nature 468 (7327): 1067-1073.
Fischer, E. S., K. Böhm, J. R. Lydeard, H. Yang, M. B. Stadler, S. Cavadini, J. Nagel, F. Serluca, V. Acker, G. M. Lingaraju, R. B. Tichkule, M. Schebesta, W. C. Forrester, M. Schirle, U. Hassiepen, J. Ottl, M. Hild, R. E. J. Beckwith, J. W. Harper, J. L. Jenkins and N. H. Thoma (2014). “Structure of the DDB1-CRBN E3 ubiquitin ligase in complex with thalidomide.” Nature 512 (7512): 49-53.
Kabsch, W. (2010). “XDS.” Acta Crystallographica Section D 66 (2): 125-132. Krissinel, E. and K. Henrick (2007). “Inference of macromolecular assemblies from crystalline state.” J Mol Biol 372 (3): 774-797.
Krissinel, E. and K. Henrick (2007). “Inference of Macromolecular Assemblies from Crystalline State.” Journal of Molecular Biology 372 (3): 774-797.
Kuriyan, J. and D. Eisenberg (2007). “The origin of protein interactions and allostery in colocalization.” Nature 450 (7172): 983-990.
Marks, B. D., N. Qadir, H. C. Eliason, M. S. Shekhani, K. Doering and K. W. Vogel (2005). “Multiparameter Analysis of a Screen for Progesterone Receptor Ligands: Comparing Fluorescence Lifetime and Fluorescence Polarization Measurements.” ASSAY and Drug Development Technologies 3 (6): 613-622.
Matyskiela, M. E., G. Lu, T. Ito, B. Pagarigan, C. C. Lu, K. Miller, W. Fang, N. Y. Wang, D. Nguyen, J. Houston, G. Carmel, T. Tran, M. Riley, L. Nosaka, G. C. Lander, S. Gaidarova, S. Xu, A. L. Ruchelman, H. Handa, J. Carmichael, T. O. Daniel, B. E. Cathers, A. Lopez-Girona and P. P. Chamberlain (2016). “A novel cereblon modulator recruits GSPT1 to the CRL4 (CRBN) ubiquitin ligase.” Nature 535 (7611): 252-257.
McAlister, G. C., D. P. Nusinow, M. P. Jedrychowski, M. Wuhr, E. L. Huttlin, B. K. Erickson, R. Rad, W. Haas and S. P. Gygi (2014). “MultiNotch MS3 enables accurate, sensitive, and multiplexed detection of differential expression across cancer cell line proteomes.” Anal Chem 86 (14): 7150-7158.
McCoy, A. J., R. W. Grosse-Kunstleve, P. D. Adams, M. D. Winn, L. C. Storoni and R. J. Read (2007). “Phaser crystallographic software.” Journal of Applied Crystallography 40 (4): 658-674.
Moriarty, N. W., R. W. Grosse-Kunstleve and P. D. Adams (2009). “electronic Ligand Builder and Optimization Workbench (eLBOW): a tool for ligand coordinate and restraint generation.” Acta Crystallographica Section D 65 (10): 1074-1080.
Morin, A., B. Eisenbraun, J. Key, P. C. Sanschagrin, M. A. Timony, M. Ottaviano and P. Sliz (2013). “Collaboration gets the most out of software.” Elife 2: e01456.
Petzold, G., E. S. Fischer and N. H. Thoma (2016). “Structural basis of lenalidomide-induced CK1alpha degradation by the CRL4 ubiquitin ligase.” Nature 532 (7597): 127-130.
Petzold, G., E. S. Fischer and N. H. Thoma (2016). “Structural basis of lenalidomide-induced CK1α degradation by the CRL4CRBN ubiquitin ligase.” Nature 532 (7597): 127-130.
Ritchie, M. E., B. Phipson, D. Wu, Y. Hu, C. W. Law, W. Shi and G. K. Smyth (2015). “limma powers differential expression analyses for RNA-sequencing and microarray studies.” Nucleic Acids Res 43 (7): e47.
Sircar, A., S. Chaudhury, K. P. Kilambi, M. Berrondo and J. J. Gray (2010). “A generalized approach to sampling backbone conformations with RosettaDock for CAPRI rounds 13-19.” Proteins 78 (15): 3115-3123.
Stathis, A., E. Zucca, M. Bekradda, C. Gomez-Roca, J. P. Delord, T. de La Motte Rouge, E. Uro-Coste, F. de Braud, G. Pelosi and C. A. French (2016). “Clinical Response of Carcinomas Harboring the BRD4-NUT Oncoprotein to the Targeted Bromodomain Inhibitor OTXO 15/MK-8628.” Cancer Discov 6 (5): 492-500.
Team, R. C. (2013). “R: A language and environment for statistical computing.” R Foundation for Statistical Computing, Vienna, Austria.
Winn, M. D., C. C. Ballard, K. D. Cowtan, E. J. Dodson, P. Emsley, P. R. Evans, R. M. Keegan, E. B. Krissinel, A. G. W. Leslie, A. McCoy, S. J. McNicholas, G. N. Murshudov, N. S. Pannu, E. A. Potterton, H. R. Powell, R. J. Read, A. Vagin and K. S. Wilson (2011). “Overview of the CCP4 suite and current developments.” Acta Crystallographica Section D 67 (4): 235-242.
Winter, G. E., D. L. Buckley, J. Paulk, J. M. Roberts, A. Souza, S. Dhe-Paganon and J. E.
Bradner (2015). “DRUG DEVELOPMENT. Phthalimide conjugation as a strategy for in vivo target protein degradation.” Science 348 (6241): 1376-1381.
Zakeri, B., J. O. Fierer, E. Celik, E. C. Chittock, U. Schwarz-Linek, V. T. Moy and M. Howarth (2012). “Peptide tag forming a rapid covalent bond to a protein, through engineering a bacterial adhesin.” Proceedings of the National Academy of Sciences 109 (12): E690-E697.
Zuber, J., J. Shi, E. Wang, A. R. Rappaport, H. Herrmann, E. A. Sison, D. Magoon, J. Qi, K. Blatt, M. Wunderlich, M. J. Taylor, C. Johns, A. Chicas, J. C. Mulloy, S. C. Kogan, P. Brown, P. Valent, J. E. Bradner, S. W. Lowe and C. R. Vakoc (2011). “RNAi screen identifies Brd4 as a therapeutic target in acute myeloid leukaemia.” Nature 478 (7370): 524-528.

1. A method for generating a dimerization and/or degradation moiety for a first protein and a second protein, the method comprising:
(a) generating a first set of poses by docking a first protein-first ligand pair structure and a second protein-second ligand pair structure in silico;
(b) generating a set of feasible poses by
(i) selecting a subset of the first set poses by scoring and
(ii) structurally clustering the subset in silico;
(c) selecting a preferred pose from the set of feasible poses based upon the relative position and orientation of the first protein-first ligand pair structure and the second protein-second ligand pair;
(d) designing a covalent linker between the first ligand and the second ligand in the preferred pose; and
(e) synthesizing, or having synthesized, the dimerization and/or degradation moiety comprising the first ligand, the second ligand, and the covalent linker.

2. The method of claim 1, further comprising experimentally measuring binding of the first protein, the second protein, and the dimerization and/or degradation moiety; or
further comprising experimentally measuring a functional result of binding the first protein, the second protein, and the dimerization and/or degradation moiety.

3. (canceled)

4. The method of claim 2, wherein the functional result comprises an enzymatic activity, chemical modification, dimerization of the first and second protein, or degradation of the first or second protein.

5. The method of claim 1, further comprising synthesizing a library of dimerization and/or degradation moieties, and further comprising experimentally screening the library of dimerization and/or degradation moiety.

6. (canceled)

7. The method of claim 1, wherein the first and second proteins do not naturally bind each other in vivo.

8. The method of claim 1, wherein the first protein or the second protein is a ubiquitin ligase, wherein the ubiquitin ligase is an E3 ubiquitin ligase or a component of an E3 ubiquitin ligase, wherein the E3 ubiquitin ligase is CRL4^{CRBN}, CRL4^{DCAF15}, CRL3^{KEAP1} or CRL2^{VHL}.

9. (canceled)

10. (canceled)

11. The method of claim 1, wherein the first protein or the second protein is an E2 ubiquitin conjugating enzyme, or
wherein the first protein or the second protein is a Von Hippel-Lindau tumor suppressor protein (VHL), or
wherein the first protein or the second protein is a subunit of a proteasome.

12. (canceled)

13. (canceled)

14. The method of claim 1, wherein the first ligand or the second ligand is a ubiquitin ligase ligand, or
wherein the first ligand or the second ligand is an E3 ubiquitin ligase ligand,
wherein the first ligand or the second ligand is thalidomide, lenalidomide, pomalidomide, or an analog or derivative thereof, or
wherein the first ligand or the second ligand is a E2 ubiquitin conjugating enzyme ligand,
wherein the first ligand or the second ligand is a Von Hippel-Lindau tumor suppressor protein (VHL) ligand, or
wherein the first ligand or the second ligand is a proteasome subunit ligand.

15. (canceled)

16. (canceled)

17. (canceled)

18. (canceled)

19. (canceled)

20. The method of claim 1, wherein step (d) further comprises calculating a shortest path between the first and second ligands.

21. The method of claim 20, where the shortest path is calculated between a centroid and/or a predetermined atom of each of the first and second ligands.

22. The method of claim 20, further comprising fitting a chemical structure to the shortest path, thereby designing the covalent linker.

23. A method for generating a dimerization and/or degradation moiety for a first protein and a second protein, the method comprising
(a) generating a first set of poses by docking a first protein structure and a second protein structure in silico;
(b) generating a set of feasible poses by
(i) selecting a subset of the first set poses by scoring and
(ii) structurally clustering the subset in silico;
(c) selecting a preferred pose from the set of feasible poses based upon the relative position and orientation of the first protein structure and the second protein structure;
(d) designing a covalent linker between a first ligand for the first protein and a second ligand for the second protein in the preferred pose; and
(e) synthesizing, or having synthesized, the dimerization and/or degradation moiety comprising the first ligand, the second ligand, and the covalent linker.

24. The method of claim 23, wherein step (d) further comprises docking a first ligand to the first protein and/or a second ligand to the second protein.

25. A method for generating a dimerization and/or degradation moiety for a first protein and a second protein, the method comprising:
(a) generating, in silico, a set of poses by docking a first protein, optionally bound to a first ligand, and a second protein, optionally bound to a second ligand, wherein:
(i) a score is calculated based on energy of interactions between the first protein and the second protein for each of the poses; and
(ii) a spatial relationship between the first protein and the second proteins is quantified for each of the poses,
(b) generating a subset of poses by selecting one or more poses from the set of poses based on the scores of the poses,
(c) identifying a candidate pose from the subset of poses based on the spatial relationship between the two proteins;
(d) designing a linker between the first ligand and the second ligand that accommodates the candidate pose; and
(e) synthesizing or having synthesized the dimerization and/or degradation moiety having the first ligand, the second ligand, and the linker.

26. The method of claim 25, wherein the dimerization and/or degradation moiety causes degradation of the first protein with a higher specificity than the binding specificity of the first ligand for the first protein.

27. The method of claim 25, wherein the spatial relationship between the first protein and the second protein is quantified by calculating the shortest path between a first set of solvent-exposed atoms on the first ligand and a second set of solvent-exposed atoms on the second ligand, or
wherein the spatial relationship between the first protein and the second protein is quantified by calculating the shortest path between the centroid of the first ligand and the centroid of the second ligands.

28. (canceled)

29. The method of claim 25, wherein the dimerization and/or degradation moiety dimerizes the first protein and the second protein in a low-energy level conformation.

30. A method as in claim 27, in which the plurality of shortest paths calculated is compiled to generate a distance profile for the subset of poses.

31. The method of claim 30, wherein the distance profile of the subset of poses has a distinct cluster of poses that have similar shortest paths.

32. The method of claim 31, wherein the candidate pose is the lowest scoring pose of the cluster of poses.

33. The method of claim 30, wherein the specificity of the dimerization and/or degradation moiety for the first protein and the second protein is predicted from the distance profile for the subset of poses.

34. The method of claim 33, wherein relative specificity the dimerization and/or degradation moiety for two different first proteins can be predictively distinguished by comparing the distance profiles for the subset of poses for each of the two different first proteins and the second protein.

35. The method of claim 25, further comprising experimentally measuring binding of the first protein, the second protein, and the dimerization and/or degradation moiety, or
further comprising experimentally measuring a functional result of binding the first protein, the second protein, and the dimerization and/or degradation moiety.

36. (canceled)

37. The method of claim 35, wherein the functional result comprises an enzymatic activity, chemical modification, dimerization of the first and second protein, or degradation of the first or second protein.

38. The method of claim 25, further comprising synthesizing a library of dimerization and/or degradation moieties, and further comprising experimentally screening the library of dimerization and/or degradation moieties.

39. The method of claim 38, further comprising experimentally screening the library of dimerization and/or degradation moieties.

40. The method of claim 25, wherein the first and second proteins do not naturally bind each other in vivo.

41. The method of claim 25, wherein the first protein or the second protein is a ubiquitin ligase,
wherein the ubiquitin ligase is an E3 ubiquitin ligase or a component of the E3 ubiquitin ligase, or
wherein the E3 ubiquitin ligase is CRL4^{CRBN}, CRL4^{DCAF15}, CRL3^{KEAP1} or CRL2^{VHL}.

42. (canceled)

43. (canceled)

44. The method of claim 41, wherein the component of the E3 ubiquitin ligase is CRBN, DCAF15, KEAP1, or VHL.

45. The method of claim 25, wherein the first protein or the second protein is an E2 ubiquitin conjugating enzyme, or
wherein the first protein or the second protein is VHL, or
wherein the first protein or the second protein is a subunit of a proteasome.

46. (canceled)

47. (canceled)

48. The method of claim 25, wherein the first ligand or the second ligand is a ubiquitin ligase ligand, or
wherein the first ligand or the second ligand is an E3 ubiquitin ligase ligand, or
wherein the first ligand or the second ligand is a ligand for a component of an E3 ubiquitin ligase, or
wherein the first ligand or the second ligand is thalidomide, lenalidomide, pomalidomide, or an analogue or derivative thereof, or
wherein the first ligand or the second ligand is a E2 ubiquitin conjugating enzyme ligand, or
wherein the first ligand or the second ligand is a Von Hippel-Lindau tumor suppressor protein (VHL) ligand, or
wherein the first ligand or the second ligand is a proteasome subunit ligand.

49. (canceled)

50. (canceled)

51. (canceled)

52. (canceled)

53. (canceled)

54. (canceled)

55. The method of claim 27, wherein the step of designing the linker further comprises fitting a chemical structure to the shortest path of the candidate pose, thereby designing the linker.

56. The method of claim 1, wherein the dimerization and/or degradation moiety comprises a heterobifunctional binder, a molecular glue, an immunomodulatory imide drug (IMiD)-like molecule/molecular glue, a cyclic peptide-like molecule, a peptide, a peptide mimetic, deoxyribonucleic acid (DNA), ribonucleic acid (RNA), a nucleic acid mimetic, and a computationally-designed mini-protein.
